SEQUENCE LISTING (37 sequences)

SEQ ID NO: 1 - protein, 822 aa, Homo sapiens [sequence omitted]

SEQ ID NO: 2 - DNA, 2936 bp, Homo sapiens
  source 1..2936: mol_type = other DNA
gtgggagagtgacacacaaaacaccccgggaagccggaggaggcgtggaaaggtggggcccgggaggtaaaactgggatcggcgcgttcagtgaggctgattaaagaacaacgcgcaaccagcgccatacggcgctcctcagggtgactcggctctctagctcttaagggttagaagcgaccctcgcacggtccccggtgccacctggagcatcataatggactctgtggtgccctctgacaagggcaactacacctgcattgtggagaatgagtacggcagcatcaaccacacataccagctggatgtcgtggagcggtcccctcaccggcccatcctgcaagcagggttgcccgccaacaaaacagtggccctgggtagcaacgtggagttcatgtgtaaggtgtacagtgacccgcagccgcacatccagtggctaaagcacatcgaggtgaatgggagcaagattggcccagacaacctgccttatgtccagatcttgaagactgctggagttaataccaccgacaaagagatggaggtgcttcacttaagaaatgtctcctttgaggacgcaggggagtatacgtgcttggcgggtaactctatcggactctcccatcactctgcatggttgaccgttctggaagccctggaagagaggccggcagtgatgacctcgcccctgtacctggagatcatcatctattgcacaggggccttcctcatctcctgcatggtggggtcggtcatcgtctacaagatgaagagtggtaccaagaagagtgacttccacagccagatggctgtgcacaagctggccaagagcatccctctgcgcagacaggtaacagtgtctgctgactccagtgcatccatgaactctggggttcttctggttcggccatcacggctctcctccagtgggactcccatgctagcaggggtctctgagtatgagcttcccgaagaccctcgctgggagctgcctcgggacagactggtcttaggcaaacccctgggagagggctgctttgggcaggtggtgttggcagaggctatcgggctggacaaggacaaacccaaccgtgtgaccaaagtggctgtgaagatgttgaagtcggacgcaacagagaaagacttgtcagacctgatctcagaaatggagatgatgaagatgatcgggaagcataagaatatcatcaacctgctgggggcctgcacgcaggatggtcccttgtatgtcatcgtggagtatgcctccaagggcaacctgcgggagtacctgcaggcccggaggcccccagggctggaatactgctacaaccccagccacaacccagaggagcagctctcctccaaggacctggtgtcctgcgcctaccaggtggcccgaggcatggagtatctggcctccaagaagtgcatacaccgagacctggcagccaggaatgtcctggtgacagaggacaatgtgatgaagatagcagactttggcctcgcacgggacattcaccacatcgactactataaaaagacaaccaacggccgactgcctgtgaagtggatggcacccgaggcattatttgaccggatctacacccaccagagtgatgtgtggtctttcggggtgctcctgtgggagatcttcactctgggcggctccccataccccggtgtgcctgtggaggaacttttcaagctgctgaaggagggtcaccgcatggacaagcccagtaactgcaccaacgagctgtacatgatgatgcgggactgctggcatgcagtgccctcacagagacccaccttcaagcagctggtggaagacctggaccgcatcgtggccttgacctccaaccaggagtacctggacctgtccatgcccctggaccagtactcccccagctttcccgacacccggagctctacgtgctcctcaggggaggattccgtcttctctcatgagccgctgcccgaggagccctgcctgccccgacacccagcccagcttgccaatggcggactcaaacgccgctgactgccacccacacgccctccccagactccaccgtcagctgtaaccctcacccacagcccctgctgggcccaccacctgtccgtccctgtcccctttcctgctggcaggagccggctgcctaccaggggccttcctgtgtggcctgccttcaccccactcagctcacctctccctccacctcctctccacctgctggtgagaggtgcaaagaggcagatctttgctgccagccacttcatcccctcccagatgttggaccaacacccctccctgccaccaggcactgcctggagggcagggagtgggagccaatgaacaggcatgcaagtgagagcttcctgagctttctcctgtcggtttggtctgttttgccttcacccataagcccctcgcactctggtggcaggtgccttgtcctcagggctacagcagtagggaggtcagtgcttcgtgcctcgattgaaggtgacctctgccccagataggtggtgccagtggcttattaattccgatactagtttgctttgctgaccaaatgcctggtaccagaggatggtgaggcgaaggccaggttgggggcagtgttgtggccctggggcccagccccaaactgggggctctgtatatagctatgaagaaaacacaaagtgtataaatctgagtatatatttacatgtctttttaaaagggtcgttaccagagatttacccatcgggtaagatgctcctggtggctgggaggcatcagttgctatatattaaaaacaaaaaagaaaaaaaaggaaaatgtttttaaaaaggtcatatattttttgctacttttgctgttttatttttttaaattatgttctaaacctattttcagtttaggtccctcaataaaaattgctgctgcttca

SEQ ID NO: 3 - DNA, 1821 bp, Homo sapiens
  source 1..1821: mol_type = other DNA
atggactctgtggtgccctctgacaagggcaactacacctgcattgtggagaatgagtacggcagcatcaaccacacataccagctggatgtcgtggagcggtcccctcaccggcccatcctgcaagcagggttgcccgccaacaaaacagtggccctgggtagcaacgtggagttcatgtgtaaggtgtacagtgacccgcagccgcacatccagtggctaaagcacatcgaggtgaatgggagcaagattggcccagacaacctgccttatgtccagatcttgaagactgctggagttaataccaccgacaaagagatggaggtgcttcacttaagaaatgtctcctttgaggacgcaggggagtatacgtgcttggcgggtaactctatcggactctcccatcactctgcatggttgaccgttctggaagccctggaagagaggccggcagtgatgacctcgcccctgtacctggagatcatcatctattgcacaggggccttcctcatctcctgcatggtggggtcggtcatcgtctacaagatgaagagtggtaccaagaagagtgacttccacagccagatggctgtgcacaagctggccaagagcatccctctgcgcagacaggtaacagtgtctgctgactccagtgcatccatgaactctggggttcttctggttcggccatcacggctctcctccagtgggactcccatgctagcaggggtctctgagtatgagcttcccgaagaccctcgctgggagctgcctcgggacagactggtcttaggcaaacccctgggagagggctgctttgggcaggtggtgttggcagaggctatcgggctggacaaggacaaacccaaccgtgtgaccaaagtggctgtgaagatgttgaagtcggacgcaacagagaaagacttgtcagacctgatctcagaaatggagatgatgaagatgatcgggaagcataagaatatcatcaacctgctgggggcctgcacgcaggatggtcccttgtatgtcatcgtggagtatgcctccaagggcaacctgcgggagtacctgcaggcccggaggcccccagggctggaatactgctacaaccccagccacaacccagaggagcagctctcctccaaggacctggtgtcctgcgcctaccaggtggcccgaggcatggagtatctggcctccaagaagtgcatacaccgagacctggcagccaggaatgtcctggtgacagaggacaatgtgatgaagatagcagactttggcctcgcacgggacattcaccacatcgactactataaaaagacaaccaacggccgactgcctgtgaagtggatggcacccgaggcattatttgaccggatctacacccaccagagtgatgtgtggtctttcggggtgctcctgtgggagatcttcactctgggcggctccccataccccggtgtgcctgtggaggaacttttcaagctgctgaaggagggtcaccgcatggacaagcccagtaactgcaccaacgagctgtacatgatgatgcgggactgctggcatgcagtgccctcacagagacccaccttcaagcagctggtggaagacctggaccgcatcgtggccttgacctccaaccaggagtacctggacctgtccatgcccctggaccagtactcccccagctttcccgacacccggagctctacgtgctcctcaggggaggattccgtcttctctcatgagccgctgcccgaggagccctgcctgccccgacacccagcccagcttgccaatggcggactcaaacgccgctga

SEQ ID NO: 4 - DNA, 21 bp, Homo sapiens
  source 1..21: mol_type = other DNA
cacctggagcatcataatgga

SEQ ID NO: 5 - DNA, 22 bp, Homo sapiens
  source 1..22: mol_type = other DNA
tggagcatcataatggactctg

SEQ ID NO: 6 - DNA, 19 bp, Homo sapiens
  source 1..19: mol_type = other DNA
agtcagcggcgtttgagtc

SEQ ID NO: 7 - DNA, 18 bp, Homo sapiens
  source 1..18: mol_type = other DNA
gtgtgggtggcagtcagc

SEQ ID NO: 8 - DNA, 19 bp, Homo sapiens
  source 1..19: mol_type = other DNA
cagtcagcggcgtttgagt

SEQ ID NO: 9 - protein, 604 aa, Homo sapiens [sequence omitted]

SEQ ID NO: 10 - DNA, 2280 bp, Homo sapiens
  source 1..2280: mol_type = other DNA
ccctggaagagaggccggcagtgatgacctcgcccctgtacctggagatcatcatctattgcacaggggccttcctcatctcctgcatggtggggtcggtcatcgtctacaagatgaagagtggtaccaagaagagtgacttccacagccagatggctgtgcacaagctggccaagagcatccctctgcgcagacaggtgtctgctgactccagtgcatccatgaactctggggttcttctggttcggccatcacggctctcctccagtgggactcccatgctagcaggggtctctgagtatgagcttcccgaagaccctcgctgggagctgcctcgggacagactggtcttaggcaaacccctgggagagggctgctttgggcaggtggtgttggcagaggctatcgggctggacaaggacaaacccaaccgtgtgaccaaagtggctgtgaagatgttgaagtcggacgcaacagagaaagacttgtcagacctgatctcagaaatggagatgatgaagatgatcgggaagcataagaatatcatcaacctgctgggggcctgcacgcaggatggtcccttgtatgtcatcgtggagtatgcctccaagggcaacctgcgggagtacctgcaggcccggaggcccccagggctggaatactgctacaaccccagccacaacccagaggagcagctctcctccaaggacctggtgtcctgcgcctaccaggtggcccgaggcatggagtatctggcctccaagaagtgcatacaccgagacctggcagccaggaatgtcctggtgacagaggacaatgtgatgaagatagcagactttggcctcgcacgggacattcaccacatcgactactataaaaagacaaccaacggccgactgcctgtgaagtggatggcacccgaggcattatttgaccggatctacacccaccagagtgatgtgtggtctttcggggtgctcctgtgggagatcttcactctgggcggctccccataccccggtgtgcctgtggaggaacttttcaagctgctgaaggagggtcaccgcatggacaagcccagtaactgcaccaacgagctgtacatgatgatgcgggactgctggcatgcagtgccctcacagagacccaccttcaagcagctggtggaagacctggaccgcatcgtggccttgacctccaaccaggagtacctggacctgtccatgcccctggaccagtactcccccagctttcccgacacccggagctctacgtgctcctcaggggaggattccgtcttctctcatgagccgctgcccgaggagccctgcctgccccgacacccagcccagcttgccaatggcggactcaaacgccgctgactgccacccacacgccctccccagactccaccgtcagctgtaaccctcacccacagcccctgctgggcccaccacctgtccgtccctgtcccctttcctgctggcaggagccggctgcctaccaggggccttcctgtgtggcctgccttcaccccactcagctcacctctccctccacctcctctccacctgctggtgagaggtgcaaagaggcagatctttgctgccagccacttcatcccctcccagatgttggaccaacacccctccctgccaccaggcactgcctggagggcagggagtgggagccaatgaacaggcatgcaagtgagagcttcctgagctttctcctgtcggtttggtctgttttgccttcacccataagcccctcgcactctggtggcaggtgccttgtcctcagggctacagcagtagggaggtcagtgcttcgtgcctcgattgaaggtgacctctgccccagataggtggtgccagtggcttattaattccgatactagtttgctttgctgaccaaatgcctggtaccagaggatggtgaggcgaaggccaggttgggggcagtgttgtggccctggggcccagccccaaactgggggctctgtatatagctatgaagaaaacacaaagtgtataaatctgagtatatatttacatgtctttttaaaagggtcgttaccagagatttacccatcgggtaagatgctcctggtggctgggaggcatcagttgctatatattaaaaacaaaaaagaaaaaaaaggaaaatgtttttaaaaaggtcatatattttttgctacttttgctgttttatttttttaaattatgttctaaacctattttcagtttaggtccctcaataaaaattgctgctgcttca

SEQ ID NO: 11 - DNA, 1359 bp, Homo sapiens
  source 1..1359: mol_type = other DNA
atgacctcgcccctgtacctggagatcatcatctattgcacaggggccttcctcatctcctgcatggtggggtcggtcatcgtctacaagatgaagagtggtaccaagaagagtgacttccacagccagatggctgtgcacaagctggccaagagcatccctctgcgcagacaggtgtctgctgactccagtgcatccatgaactctggggttcttctggttcggccatcacggctctcctccagtgggactcccatgctagcaggggtctctgagtatgagcttcccgaagaccctcgctgggagctgcctcgggacagactggtcttaggcaaacccctgggagagggctgctttgggcaggtggtgttggcagaggctatcgggctggacaaggacaaacccaaccgtgtgaccaaagtggctgtgaagatgttgaagtcggacgcaacagagaaagacttgtcagacctgatctcagaaatggagatgatgaagatgatcgggaagcataagaatatcatcaacctgctgggggcctgcacgcaggatggtcccttgtatgtcatcgtggagtatgcctccaagggcaacctgcgggagtacctgcaggcccggaggcccccagggctggaatactgctacaaccccagccacaacccagaggagcagctctcctccaaggacctggtgtcctgcgcctaccaggtggcccgaggcatggagtatctggcctccaagaagtgcatacaccgagacctggcagccaggaatgtcctggtgacagaggacaatgtgatgaagatagcagactttggcctcgcacgggacattcaccacatcgactactataaaaagacaaccaacggccgactgcctgtgaagtggatggcacccgaggcattatttgaccggatctacacccaccagagtgatgtgtggtctttcggggtgctcctgtgggagatcttcactctgggcggctccccataccccggtgtgcctgtggaggaacttttcaagctgctgaaggagggtcaccgcatggacaagcccagtaactgcaccaacgagctgtacatgatgatgcgggactgctggcatgcagtgccctcacagagacccaccttcaagcagctggtggaagacctggaccgcatcgtggccttgacctccaaccaggagtacctggacctgtccatgcccctggaccagtactcccccagctttcccgacacccggagctctacgtgctcctcaggggaggattccgtcttctctcatgagccgctgcccgaggagccctgcctgccccgacacccagcccagcttgccaatggcggactcaaacgccgctga

SEQ ID NO: 12 - DNA, 18 bp, Homo sapiens
  source 1..18: mol_type = other DNA
ccggcagtgatgacctcg

SEQ ID NO: 13 - DNA, 18 bp, Homo sapiens
  source 1..18: mol_type = other DNA
cagtgatgacctcgcccc

SEQ ID NO: 14 - protein, 462 aa, Homo sapiens
MTSPLYLEIIIYCTGAFLISCMVGSVIVYKMKSGTKKSDFHSQMAVHKLAKSIPLRRQVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQVLHPDLTTHLLPLRCFWKVRKRRCLLRGQEPHPCSRGAGQRVKAQQPSTMDGFLQGNRWGWAGEGAPT

SEQ ID NO: 15 - DNA, 2913 bp, Homo sapiens
  source 1..2913: mol_type = other DNA
ccgtagctccatattggacatccccagaaaagatggaaaagaaattgcatgcagtgccggctgccaagacagtgaagttcaaatgcccttccagtgggaccccaaaccccacactgcgctggttgaaaaatggcaaagaattcaaacctgaccacagaattggaggctacaaggtccgttatgccacctggagcatcataatggactctgtggtgccctctgacaagggcaactacacctgcattgtggagaatgagtacggcagcatcaaccacacataccagctggatgtcgtggagcggtcccctcaccggcccatcctgcaagcagggttgcccgccaacaaaacagtggccctgggtagcaacgtggagttcatgtgtaaggtgtacagtgacccgcagccgcacatccagtggctaaagcacatcgaggtgaatgggagcaagattggcccagacaacctgccttatgtccagatcttgaagactgctggagttaataccaccgacaaagagatggaggtgcttcacttaagaaatgtctcctttgaggacgcaggggagtatacgtgcttggcgggtaactctatcggactctcccatcactctgcatggttgaccgttctggaagccctggaagagaggccggcagtgatgacctcgcccctgtacctggagatcatcatctattgcacaggggccttcctcatctcctgcatggtggggtcggtcatcgtctacaagatgaagagtggtaccaagaagagtgacttccacagccagatggctgtgcacaagctggccaagagcatccctctgcgcagacaggtgtctgctgactccagtgcatccatgaactctggggttcttctggttcggccatcacggctctcctccagtgggactcccatgctagcaggggtctctgagtatgagcttcccgaagaccctcgctgggagctgcctcgggacagactggtcttaggcaaacccctgggagagggctgctttgggcaggtggtgttggcagaggctatcgggctggacaaggacaaacccaaccgtgtgaccaaagtggctgtgaagatgttgaagtcggacgcaacagagaaagacttgtcagacctgatctcagaaatggagatgatgaagatgatcgggaagcataagaatatcatcaacctgctgggggcctgcacgcaggatggtcccttgtatgtcatcgtggagtatgcctccaagggcaacctgcgggagtacctgcaggcccggaggcccccagggctggaatactgctacaaccccagccacaacccagaggagcagctctcctccaaggacctggtgtcctgcgcctaccaggtggcccgaggcatggagtatctggcctccaagaagtgcatacaccgagacctggcagccaggaatgtcctggtgacagaggacaatgtgatgaagatagcagactttggcctcgcacgggacattcaccacatcgactactataaaaagacaaccaacggccgactgcctgtgaagtggatggcacccgaggcattatttgaccggatctacacccaccagagtgatgtgtggtctttcggggtgctcctgtgggagatcttcactctgggcggctccccataccccggtgtgcctgtggaggaacttttcaagctgctgaaggagggtcaccgcatggacaagcccagtaactgcaccaacgagctgtacatgatgatgcgggactgctggcatgcagtgccctcacagagacccaccttcaagcagctggtggaagacctggaccgcatcgtggccttgacctccaaccaggagtacctggacctgtccatgcccctggaccagtactcccccagctttcccgacacccggagctctacgtgctcctcaggggaggattccgtcttctctcatgagccgctgcccgaggagccctgcctgccccgacacccagcccagcttgccaatggcggactcaaacgccgctgactgccacccacacgccctccccagactccaccgtcagctgtaaccctcacccacagcccctgctgggcccaccacctgtccgtccctgtcccctttcctgctggcaggagccggctgcctaccaggggccttcctgtgtggcctgccttcaccccactcagctcacctctccctccacctcctctccacctgctggtgagaggtgcaaagaggcagatctttgctgccagccacttcatcccctcccagatgttggaccaacacccctccctgccaccaggcactgcctggagggcagggagtgggagccaatgaacaggcatgcaagtgagagcttcctgagctttctcctgtcggtttggtctgttttgccttcacccataagcccctcgcactctggtggcaggtgccttgtcctcagggctacagcagtagggaggtcagtgcttcgtgcctcgattgaaggtgacctctgccccagataggtggtgccagtggcttattaattccgatactagtttgctttgctgaccaaatgcctggtaccagaggatggtgaggcgaaggccaggttgggggcagtgttgtggccctggggcccagccccaaactgggggctctgtatatagctatgaagaaaacacaaagtgtataaatctgagtatatatttacatgtctttttaaaagggtcgttaccagagatttacccatcgggtaagatgctcctggtggctgggaggcatcagttgctatatattaaaaacaaaaaagaaaaaaaaggaaaatgtttttaaaaaggtcatatattttttgctacttttgctgttttatttttttaaattatgttctaaacctattttcagtttaggtccctcaataaaaattgctgctgcttca

SEQ ID NO: 16 - DNA, 1983 bp, Homo sapiens
  source 1..1983: mol_type = other DNA
atggaaaagaaattgcatgcagtgccggctgccaagacagtgaagttcaaatgcccttccagtgggaccccaaaccccacactgcgctggttgaaaaatggcaaagaattcaaacctgaccacagaattggaggctacaaggtccgttatgccacctggagcatcataatggactctgtggtgccctctgacaagggcaactacacctgcattgtggagaatgagtacggcagcatcaaccacacataccagctggatgtcgtggagcggtcccctcaccggcccatcctgcaagcagggttgcccgccaacaaaacagtggccctgggtagcaacgtggagttcatgtgtaaggtgtacagtgacccgcagccgcacatccagtggctaaagcacatcgaggtgaatgggagcaagattggcccagacaacctgccttatgtccagatcttgaagactgctggagttaataccaccgacaaagagatggaggtgcttcacttaagaaatgtctcctttgaggacgcaggggagtatacgtgcttggcgggtaactctatcggactctcccatcactctgcatggttgaccgttctggaagccctggaagagaggccggcagtgatgacctcgcccctgtacctggagatcatcatctattgcacaggggccttcctcatctcctgcatggtggggtcggtcatcgtctacaagatgaagagtggtaccaagaagagtgacttccacagccagatggctgtgcacaagctggccaagagcatccctctgcgcagacaggtgtctgctgactccagtgcatccatgaactctggggttcttctggttcggccatcacggctctcctccagtgggactcccatgctagcaggggtctctgagtatgagcttcccgaagaccctcgctgggagctgcctcgggacagactggtcttaggcaaacccctgggagagggctgctttgggcaggtggtgttggcagaggctatcgggctggacaaggacaaacccaaccgtgtgaccaaagtggctgtgaagatgttgaagtcggacgcaacagagaaagacttgtcagacctgatctcagaaatggagatgatgaagatgatcgggaagcataagaatatcatcaacctgctgggggcctgcacgcaggatggtcccttgtatgtcatcgtggagtatgcctccaagggcaacctgcgggagtacctgcaggcccggaggcccccagggctggaatactgctacaaccccagccacaacccagaggagcagctctcctccaaggacctggtgtcctgcgcctaccaggtggcccgaggcatggagtatctggcctccaagaagtgcatacaccgagacctggcagccaggaatgtcctggtgacagaggacaatgtgatgaagatagcagactttggcctcgcacgggacattcaccacatcgactactataaaaagacaaccaacggccgactgcctgtgaagtggatggcacccgaggcattatttgaccggatctacacccaccagagtgatgtgtggtctttcggggtgctcctgtgggagatcttcactctgggcggctccccataccccggtgtgcctgtggaggaacttttcaagctgctgaaggagggtcaccgcatggacaagcccagtaactgcaccaacgagctgtacatgatgatgcgggactgctggcatgcagtgccctcacagagacccaccttcaagcagctggtggaagacctggaccgcatcgtggccttgacctccaaccaggagtacctggacctgtccatgcccctggaccagtactcccccagctttcccgacacccggagctctacgtgctcctcaggggaggattccgtcttctctcatgagccgctgcccgaggagccctgcctgccccgacacccagcccagcttgccaatggcggactcaaacgccgctga

SEQ ID NO: 17 - DNA, 20 bp, Homo sapiens
  source 1..20: mol_type = other DNA
ttggacatccccagaaaaga

SEQ ID NO: 18 - DNA, 20 bp, Homo sapiens
  source 1..20: mol_type = other DNA
ccatattggacatccccaga

SEQ ID NO: 19 - protein, 660 aa, Homo sapiens
MEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEERPAVMTSPLYLEIIIYCTGAFLISCMVGSVIVYKMKSGTKKSDFHSQMAVHKLAKSIPLRRQVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSMPLDQYSPSFPDTRSSTCSSGEDSVFSHEPLPEEPCLPRHPAQLANGGLKRR

SEQ ID NO: 20 - DNA, 2431 bp, Homo sapiens
  source 1..2431: mol_type = other DNA
actgctggagttaataccaccgacaaagagatggaggtgcttcacttaagaaatgtctcctttgaggacgcaggggagtatacgtgcttggcgggtaactctatcggactctcccatcactctgcatggttgaccgttctggaagccctggaagagaggccggcagtgatgacctcgcccctgtacctggagatcatcatctattgcacaggggccttcctcatctcctgcatggtggggtcggtcatcgtctacaagatgaagagtggtaccaagaagagtgacttccacagccagatggctgtgcacaagctggccaagagcatccctctgcgcagacaggtaacagtgtctgctgactccagtgcatccatgaactctggggttcttctggttcggccatcacggctctcctccagtgggactcccatgctagcaggggtctctgagtatgagcttcccgaagaccctcgctgggagctgcctcgggacagactggtcttaggcaaacccctgggagagggctgctttgggcaggtggtgttggcagaggctatcgggctggacaaggacaaacccaaccgtgtgaccaaagtggctgtgaagatgttgaagtcggacgcaacagagaaagacttgtcagacctgatctcagaaatggagatgatgaagatgatcgggaagcataagaatatcatcaacctgctgggggcctgcacgcaggatggtcccttgtatgtcatcgtggagtatgcctccaagggcaacctgcgggagtacctgcaggcccggaggcccccagggctggaatactgctacaaccccagccacaacccagaggagcagctctcctccaaggacctggtgtcctgcgcctaccaggtggcccgaggcatggagtatctggcctccaagaagtgcatacaccgagacctggcagccaggaatgtcctggtgacagaggacaatgtgatgaagatagcagactttggcctcgcacgggacattcaccacatcgactactataaaaagacaaccaacggccgactgcctgtgaagtggatggcacccgaggcattatttgaccggatctacacccaccagagtgatgtgtggtctttcggggtgctcctgtgggagatcttcactctgggcggctccccataccccggtgtgcctgtggaggaacttttcaagctgctgaaggagggtcaccgcatggacaagcccagtaactgcaccaacgagctgtacatgatgatgcgggactgctggcatgcagtgccctcacagagacccaccttcaagcagctggtggaagacctggaccgcatcgtggccttgacctccaaccaggagtacctggacctgtccatgcccctggaccagtactcccccagctttcccgacacccggagctctacgtgctcctcaggggaggattccgtcttctctcatgagccgctgcccgaggagccctgcctgccccgacacccagcccagcttgccaatggcggactcaaacgccgctgactgccacccacacgccctccccagactccaccgtcagctgtaaccctcacccacagcccctgctgggcccaccacctgtccgtccctgtcccctttcctgctggcaggagccggctgcctaccaggggccttcctgtgtggcctgccttcaccccactcagctcacctctccctccacctcctctccacctgctggtgagaggtgcaaagaggcagatctttgctgccagccacttcatcccctcccagatgttggaccaacacccctccctgccaccaggcactgcctggagggcagggagtgggagccaatgaacaggcatgcaagtgagagcttcctgagctttctcctgtcggtttggtctgttttgccttcacccataagcccctcgcactctggtggcaggtgccttgtcctcagggctacagcagtagggaggtcagtgcttcgtgcctcgattgaaggtgacctctgccccagataggtggtgccagtggcttattaattccgatactagtttgctttgctgaccaaatgcctggtaccagaggatggtgaggcgaaggccaggttgggggcagtgttgtggccctggggcccagccccaaactgggggctctgtatatagctatgaagaaaacacaaagtgtataaatctgagtatatatttacatgtctttttaaaagggtcgttaccagagatttacccatcgggtaagatgctcctggtggctgggaggcatcagttgctatatattaaaaacaaaaaagaaaaaaaaggaaaatgtttttaaaaaggtcatatattttttgctacttttgctgttttatttttttaaattatgttctaaacctattttcagtttaggtccctcaataaaaattgctgctgcttca

SEQ ID NO: 21 - DNA, 1503 bp, Homo sapiens
  source 1..1503: mol_type = other DNA
atggaggtgcttcacttaagaaatgtctcctttgaggacgcaggggagtatacgtgcttggcgggtaactctatcggactctcccatcactctgcatggttgaccgttctggaagccctggaagagaggccggcagtgatgacctcgcccctgtacctggagatcatcatctattgcacaggggccttcctcatctcctgcatggtggggtcggtcatcgtctacaagatgaagagtggtaccaagaagagtgacttccacagccagatggctgtgcacaagctggccaagagcatccctctgcgcagacaggtaacagtgtctgctgactccagtgcatccatgaactctggggttcttctggttcggccatcacggctctcctccagtgggactcccatgctagcaggggtctctgagtatgagcttcccgaagaccctcgctgggagctgcctcgggacagactggtcttaggcaaacccctgggagagggctgctttgggcaggtggtgttggcagaggctatcgggctggacaaggacaaacccaaccgtgtgaccaaagtggctgtgaagatgttgaagtcggacgcaacagagaaagacttgtcagacctgatctcagaaatggagatgatgaagatgatcgggaagcataagaatatcatcaacctgctgggggcctgcacgcaggatggtcccttgtatgtcatcgtggagtatgcctccaagggcaacctgcgggagtacctgcaggcccggaggcccccagggctggaatactgctacaaccccagccacaacccagaggagcagctctcctccaaggacctggtgtcctgcgcctaccaggtggcccgaggcatggagtatctggcctccaagaagtgcatacaccgagacctggcagccaggaatgtcctggtgacagaggacaatgtgatgaagatagcagactttggcctcgcacgggacattcaccacatcgactactataaaaagacaaccaacggccgactgcctgtgaagtggatggcacccgaggcattatttgaccggatctacacccaccagagtgatgtgtggtctttcggggtgctcctgtgggagatcttcactctgggcggctccccataccccggtgtgcctgtggaggaacttttcaagctgctgaaggagggtcaccgcatggacaagcccagtaactgcaccaacgagctgtacatgatgatgcgggactgctggcatgcagtgccctcacagagacccaccttcaagcagctggtggaagacctggaccgcatcgtggccttgacctccaaccaggagtacctggacctgtccatgcccctggaccagtactcccccagctttcccgacacccggagctctacgtgctcctcaggggaggattccgtcttctctcatgagccgctgcccgaggagccctgcctgccccgacacccagcccagcttgccaatggcggactcaaacgccgctga

SEQ ID NO: 22 - DNA, 20 bp, Homo sapiens
  source 1..20: mol_type = other DNA
ccgacaaagagatggaggtg

SEQ ID NO: 23 - DNA, 22 bp, Homo sapiens
  source 1..22: mol_type = other DNA
caaagagatggaggtgcttcac

SEQ ID NO: 24 - protein, 452 aa, Homo sapiens [sequence omitted]

SEQ ID NO: 25 - DNA, 1296 bp, Homo sapiens
  source 1..1296: mol_type = other DNA
atggtggggtcggtcatcgtctacaagatgaagagtggtaccaagaagagtgacttccacagccagatggctgtgcacaagctggccaagagcatccctctgcgcagacaggtgtctgctgactccagtgcatccatgaactctggggttcttctggttcggccatcacggctctcctccagtgggactcccatgctagcaggggtctctgagtatgagcttcccgaagaccctcgctgggagctgcctcgggacagactggtcttaggcaaacccctgggagagggctgctttgggcaggtggtgttggcagaggctatcgggctggacaaggacaaacccaaccgtgtgaccaaagtggctgtgaagatgttgaagtcggacgcaacagagaaagacttgtcagacctgatctcagaaatggagatgatgaagatgatcgggaagcataagaatatcatcaacctgctgggggcctgcacgcaggatggtcccttgtatgtcatcgtggagtatgcctccaagggcaacctgcgggagtacctgcaggcccggaggcccccagggctggaatactgctacaaccccagccacaacccagaggagcagctctcctccaaggacctggtgtcctgcgcctaccaggtggcccgaggcatggagtatctggcctccaagaagtgcatacaccgagacctggcagccaggaatgtcctggtgacagaggacaatgtgatgaagatagcagactttggcctcgcacgggacattcaccacatcgactactataaaaagacaaccaacggccgactgcctgtgaagtggatggcacccgaggcattatttgaccggatctacacccaccagagtgatgtgtggtctttcggggtgctcctgtgggagatcttcactctgggcggctccccataccccggtgtgcctgtggaggaacttttcaagctgctgaaggagggtcaccgcatggacaagcccagtaactgcaccaacgagctgtacatgatgatgcgggactgctggcatgcagtgccctcacagagacccaccttcaagcagctggtggaagacctggaccgcatcgtggccttgacctccaaccaggagtacctggacctgtccatgcccctggaccagtactcccccagctttcccgacacccggagctctacgtgctcctcaggggaggattccgtcttctctcatgagccgctgcccgaggagccctgcctgccccgacacccagcccagcttgccaatggcggactcaaacgccgctga

SEQ ID NO: 26 - protein, 431 aa, Homo sapiens [sequence omitted]

SEQ ID NO: 27 - DNA, 17 bp, Homo sapiens
  source 1..17: mol_type = other DNA
atggtggggtcggtcat

SEQ ID NO: 28 - DNA, 1269 bp, Homo sapiens
  source 1..1269: mol_type = other DNA
atgaagagtggtaccaagaagagtgacttccacagccagatggctgtgcacaagctggccaagagcatccctctgcgcagacaggtgtctgctgactccagtgcatccatgaactctggggttcttctggttcggccatcacggctctcctccagtgggactcccatgctagcaggggtctctgagtatgagcttcccgaagaccctcgctgggagctgcctcgggacagactggtcttaggcaaacccctgggagagggctgctttgggcaggtggtgttggcagaggctatcgggctggacaaggacaaacccaaccgtgtgaccaaagtggctgtgaagatgttgaagtcggacgcaacagagaaagacttgtcagacctgatctcagaaatggagatgatgaagatgatcgggaagcataagaatatcatcaacctgctgggggcctgcacgcaggatggtcccttgtatgtcatcgtggagtatgcctccaagggcaacctgcgggagtacctgcaggcccggaggcccccagggctggaatactgctacaaccccagccacaacccagaggagcagctctcctccaaggacctggtgtcctgcgcctaccaggtggcccgaggcatggagtatctggcctccaagaagtgcatacaccgagacctggcagccaggaatgtcctggtgacagaggacaatgtgatgaagatagcagactttggcctcgcacgggacattcaccacatcgactactataaaaagacaaccaacggccgactgcctgtgaagtggatggcacccgaggcattatttgaccggatctacacccaccagagtgatgtgtggtctttcggggtgctcctgtgggagatcttcactctgggcggctccccataccccggtgtgcctgtggaggaacttttcaagctgctgaaggagggtcaccgcatggacaagcccagtaactgcaccaacgagctgtacatgatgatgcgggactgctggcatgcagtgccctcacagagacccaccttcaagcagctggtggaagacctggaccgcatcgtggccttgacctccaaccaggagtacctggacctgtccatgcccctggaccagtactcccccagctttcccgacacccggagctctacgtgctcctcaggggaggattccgtcttctctcatgagccgctgcccgaggagccctgcctgccccgacacccagcccagcttgccaatggcggactcaaacgccgctga

SEQ ID NO: 29 - protein, 422 aa, Homo sapiens
MKSGTKKSDFHSQMAVHKLAKSIPLRRQVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSMPLDQYSPSFPDTRSSTCSSGEDSVFSHEPLPEEPCLPRHPAQLANGGLKRR

SEQ ID NO: 30 - DNA, 25 bp, Homo sapiens
  source 1..25: mol_type = other DNA
atgaagagtggtaccaagaagagtg

SEQ ID NO: 31 - DNA, 1104 bp, Homo sapiens
  source 1..1104: mol_type = other DNA
atgctagcaggggtctctgagtatgagcttcccgaagaccctcgctgggagctgcctcgggacagactggtcttaggcaaacccctgggagagggctgctttgggcaggtggtgttggcagaggctatcgggctggacaaggacaaacccaaccgtgtgaccaaagtggctgtgaagatgttgaagtcggacgcaacagagaaagacttgtcagacctgatctcagaaatggagatgatgaagatgatcgggaagcataagaatatcatcaacctgctgggggcctgcacgcaggatggtcccttgtatgtcatcgtggagtatgcctccaagggcaacctgcgggagtacctgcaggcccggaggcccccagggctggaatactgctacaaccccagccacaacccagaggagcagctctcctccaaggacctggtgtcctgcgcctaccaggtggcccgaggcatggagtatctggcctccaagaagtgcatacaccgagacctggcagccaggaatgtcctggtgacagaggacaatgtgatgaagatagcagactttggcctcgcacgggacattcaccacatcgactactataaaaagacaaccaacggccgactgcctgtgaagtggatggcacccgaggcattatttgaccggatctacacccaccagagtgatgtgtggtctttcggggtgctcctgtgggagatcttcactctgggcggctccccataccccggtgtgcctgtggaggaacttttcaagctgctgaaggagggtcaccgcatggacaagcccagtaactgcaccaacgagctgtacatgatgatgcgggactgctggcatgcagtgccctcacagagacccaccttcaagcagctggtggaagacctggaccgcatcgtggccttgacctccaaccaggagtacctggacctgtccatgcccctggaccagtactcccccagctttcccgacacccggagctctacgtgctcctcaggggaggattccgtcttctctcatgagccgctgcccgaggagccctgcctgccccgacacccagcccagcttgccaatggcggactcaaacgccgctga

SEQ ID NO: 32 - protein, 367 aa, Homo sapiens
MLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSMPLDQYSPSFPDTRSSTCSSGEDSVFSHEPLPEEPCLPRHPAQLANGGLKRR

SEQ ID NO: 33 - DNA, 19 bp, Homo sapiens
  source 1..19: mol_type = other DNA
catgctagcaggggtctct

SEQ ID NO: 34 - DNA, 1104 bp, Homo sapiens; identical to SEQ ID NO: 31
  source 1..1104: mol_type = other DNA

SEQ ID NO: 35 - protein, 308 aa, Homo sapiens
MLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSMPLDQYSPSFPDTRSSTCSSGEDSVFSHEPLPEEPCLPRHPAQLANGGLKRR

SEQ ID NO: 36 - DNA, 20 bp, Homo sapiens
  source 1..20: mol_type = other DNA
atgctagcaggggtctctga

SEQ ID NO: 37 - DNA, 2463 bp, Homo sapiens
  source 1..2463: mol_type = other DNA
atgtggagctggaagtgcctcctcttctgggctgtgctggtcacagccacactctgcaccgctaggccgtccccgaccttgcctgaacaagcccagccctggggagcccctgtggaagtggagtccttcctggtccaccccggtgacctgctgcagcttcgctgtcggctgcgggacgatgtgcagagcatcaactggctgcgggacggggtgcagctggcggaaagcaaccgcacccgcatcacaggggaggaggtggaggtgcaggactccgtgcccgcagactccggcctctatgcttgcgtaaccagcagcccctcgggcagtgacaccacctacttctccgtcaatgtttcagatgctctcccctcctcggaggatgatgatgatgatgatgactcctcttcagaggagaaagaaacagataacaccaaaccaaaccccgtagctccatattggacatccccagaaaagatggaaaagaaattgcatgcagtgccggctgccaagacagtgaagttcaaatgcccttccagtgggaccccaaaccccacactgcgctggttgaaaaatggcaaagaattcaaacctgaccacagaattggaggctacaaggtccgttatgccacctggagcatcataatggactctgtggtgccctctgacaagggcaactacacctgcattgtggagaatgagtacggcagcatcaaccacacataccagctggatgtcgtggagcggtcccctcaccggcccatcctgcaagcagggttgcccgccaacaaaacagtggccctgggtagcaacgtggagttcatgtgtaaggtgtacagtgacccgcagccgcacatccagtggctaaagcacatcgaggtgaatgggagcaagattggcccagacaacctgccttatgtccagatcttgaagactgctggagttaataccaccgacaaagagatggaggtgcttcacttaagaaatgtctcctttgaggacgcaggggagtatacgtgcttggcgggtaactctatcggactctcccatcactctgcatggttgaccgttctggaagccctggaagagaggccggcagtgatgacctcgcccctgtacctggagatcatcatctattgcacaggggccttcctcatctcctgcatggtggggtcggtcatcgtctacaagatgaagagtggtaccaagaagagtgacttccacagccagatggctgtgcacaagctggccaagagcatccctctgcgcagacaggtaacagtgtctgctgactccagtgcatccatgaactctggggttcttctggttcggccatcacggctctcctccagtgggactcccatgctagcaggggtctctgagtatgagcttcccgaagaccctcgctgggagctgcctcgggacagactggtcttaggcaaacccctgggagagggctgctttgggcaggtggtgttggcagaggctatcgggctggacaaggacaaacccaaccgtgtgaccaaagtggctgtgaagatgttgaagtcggacgcaacagagaaagacttgtcagacctgatctcagaaatggagatgatgaagatgatcgggaagcataagaatatcatcaacctgctgggggcctgcacgcaggatggtcccttgtatgtcatcgtggagtatgcctccaagggcaacctgcgggagtacctgcaggcccggaggcccccagggctggaatactgctacaaccccagccacaacccagaggagcagctctcctccaaggacctggtgtcctgcgcctaccaggtggcccgaggcatggagtatctggcctccaagaagtgcatacaccgagacctggcagccaggaatgtcctggtgacagaggacaatgtgatgaagatagcagactttggcctcgcacgggacattcaccacatcgactactataaaaagacaaccaacggccgactgcctgtgaagtggatggcacccgaggcattatttgaccggatctacacccaccagagtgatgtgtggtctttcggggtgctcctgtgggagatcttcactctgggcggctccccataccccggtgtgcctgtggaggaacttttcaagctgctgaaggagggtcaccgcatggacaagcccagtaactgcaccaacgagctgtacatgatgatgcgggactgctggcatgcagtgccctcacagagacccaccttcaagcagctggtggaagacctggaccgcatcgtggccttgacctccaaccaggagtacctggacctgtccatgcccctggaccagtactcccccagctttcccgacacccggagctctacgtgctcctcaggggaggattccgtcttctctcatgagccgctgcccgaggagccctgcctgccccgacacccagcccagcttgccaatggcggactcaaacgccgctga

(19) United States
(12) Patent Application Publication
(10) Pub. No.: US 2024/0327928 A1
(43) Pub. Date: Oct. 3, 2024

(54) METHOD FOR IDENTIFYING RESPONSIVENESS TO FIBROBLAST GROWTH FACTOR RECEPTOR 1 INHIBITOR THERAPY

(71) Applicant: UNIVERSITÄT ZU KÖLN, Köln (DE)

(72) Inventors: Florian MALCHERS, Köln (DE); Roman THOMAS, Köln (DE)

(21) Appl. No.: 18/681,429

(22) PCT Filed: Aug. 11, 2022
(86) PCT No.: PCT/EP2022/072607

§ 371 Date: Feb. 5, 2024

(30) Foreign Application Priority Data
Aug. 11, 2021 (EP) 21190726.6

(51) Int. Cl. C12Q 1/6886

(52) U.S. Cl. CPC C12Q 1/6886; C12Q 2600/106; C12Q 2600/112; C12Q 2600/156

(57) ABSTRACT

The present invention pertains to a method for identifying a subject suffering from cancer as a responder or non-responder to fibroblast growth factor receptor 1 (FGFR1) inhibitor therapy. The method involves the determination of the presence or absence of a genetic alteration of the FGFR1 gene, wherein the genetic variation is a copy number variation caused by a Breakage-Fusion-Bridge-like (BFB-like) mechanism wherein the break is located within, or within close proximity of, the ORF of the FGFR1 gene. These breaks associate with FGFR inhibitor sensitivity, and intra-chromosomal tail-to-tail breaks in FGFR1 may work as a predictive therapeutic marker for an FGFR inhibitor therapy in cancer that can be used to stratify patients for FGFR-inhibitor therapy. Furthermore, provided are inhibitors of FGFR1 for use in the treatment of cancer, and a method for identifying a genetic biomarker associated with a disorder, such as cancer.

### FIELD OF THE INVENTION

The present invention pertains to a method for identifying a subject suffering from cancer as a responder or non-responder to fibroblast growth factor receptor 1 (FGFR1) inhibitor therapy. The method involves the determination of the presence or absence of a genetic alteration of the FGFR1 gene, wherein the genetic alteration is a copy number variation caused by chromosomal breaks. Breakage-Fusion-Bridge-like (BFB-like) mechanism is the main cause of these copy number variations and induce breaks within, or within close proximity of, the ORF of the FGFR1 gene. The breaks lead to deregulated FGFR1 expression or to the deletion of the canonical FGFR1 ATG start-codon, are associated with FGFR inhibitor sensitivity, and intra-chromosomal tail-to-tail breaks in or close to FGFR1 and may work as a predictive therapeutic marker for an FGFR inhibitor therapy in cancer that can be used to stratify patients for FGFR-inhibitor therapy. Furthermore provided are inhibitors of FGFR1 for use in the treatment of cancer, and a method for identifying a genetic biomarker associated with a disorder, such as cancer.

### DESCRIPTION

The Fibroblast growth factor receptor 1 (FGFR) family of receptor tyrosine kinases comprises five highly conserved members, namely Fibroblast growth factor receptor 1 (FGFR1), Fibroblast growth factor receptor 2 (FGFR2), Fibroblast growth factor receptor 3 (FGFR3), Fibroblast growth factor receptor 4 (FGFR4), and Fibroblast growth factor receptor-like 1 (FGFRL1). A full-length representative protein member of the FGFR family consists of an extracellular region, composed of two or three immunoglobulin-like domains, a single hydrophobic membrane-spanning segment and a cytoplasmic tyrosine kinase domain. The extracellular portion of the protein interacts with fibroblast growth factors, setting in motion a cascade of downstream signals.

FGFR1 is also known as fms-related tyrosine kinase-2 and CD331, and the FGFR1 gene is located on human chromosome 8 at position p11.23 (i.e. 8p11.23). The gene comprises 24 exons, and codes for a precursor mRNA that is alternatively spliced at exons 8A or 8B, thereby generating two mRNAs coding for two different FGFR1 isoforms, FGFR1-IIIb (also termed FGFR1b) and FGFR1-IIIc (also termed FGFR1c), respectively. While FGFR1-IIIc appears responsible for most of the functions of the FGFR1 gene, FGFR1-IIIb appears to have only a minor, somewhat redundant functional role. Furthermore, the expression of at least nine FGFR1 splice variants are described (Gene: FGFR1 (ENSG00000077782)-Homo_sapiens-Ensembl genome browser 103). Of note, it is likely that during embryonal development, some cells express only the extracellular part of FGFR. Interestingly, the secretion of this part functions as an FGF trap (FGF is the activating ligand of FGFR1) and regulates FGFR-signalling by a FGF-gradient.

Previous studies have disclosed an association between an enhanced expression of FGFR1 and various cancers, such as breast, bladder and lung cancer. Also, somatic mutations and epigenetic changes in the expression of the FGFR1 gene have been found to be linked to various cancers, including glioblastoma, melanoma, breast, hematological and lung cancer. The FGFR1 gene is often activated in human cancers as a result of duplication, fusion with other genes, and point mutations. Accordingly, FGFR1 is classified as proto-oncogene. It is also known that several cancerous diseases are, in principle, dependent on FGFR1. In view of the above, FGFR inhibitors can be a viable therapeutic option in the treatment of cancer patients with gene amplification or increased expression of members of FGFR1. Currently, multiple FGFR inhibitors are in clinical trials.

US 20160090633A1 discloses methods for identifying a cancer patient that will be responsive to treatment with a fibroblast growth factor receptor (FGFR) inhibitor and methods of treating cancer patients. The methods involve evaluating a biological sample from the patient for the presence of one or more FGFR mutants from a FGFR mutant gene panel.

EP2695950A1 discloses a method for predicting the responsiveness of a mammalian tumor cell or cancer cell to an FGFR inhibitor comprising the evaluation of the status of FGFR and the status of c-myc, whereby the status of FGFR and of c-myc is indicative for the responsiveness to the inhibitor.

EP3102705A2 discloses a method of characterizing a cancer by obtaining a sample from a subject suspected of having cancer; and determining whether a fibroblast growth factor receptor (FGFR) fusion is present in the sample, wherein the FGFR fusion comprises a FGFR locus, thereby characterizing the cancer based on the presence or absence of the FGFR fusion.

Lung cancer is classified into two diverse types of cancer, namely non-small-cell lung carcinoma (NSCLC) and small-cell lung carcinoma (SCLC). The term NSCLC comprises any type of epithelial lung cancer other than SCLC, and accounts for about 85% of all lung cancers. Unfortunately, most cases of NSCLCs are relatively insensitive to chemotherapy, compared to SCLC. The most common types of NSCLC are squamous cell lung cancer (SQLC), large-cell carcinoma, and adenocarcinoma.

Squamous cell lung cancer (SQLC) is the second most common lung cancer subtype and among the cancers with the worst prognosis. Unfortunately, genetically activated kinase targets that provide opportunities for effective drug treatment, such as those occurring in lung adenocarcinoma (e.g., EGFR mutations or ALK rearrangements) have not been identified so far in squamous cell lung cancer. The discovery of recurrent FGFR1 amplifications (located on 8p11.23) had raised hopes that patients with such amplifications might benefit from FGFR inhibition. In a clinical trial, only a minority of patients (approx. 10%) of patients with FGFR1-amplified tumors exhibited durable responses to single agent FGFR inhibition. Due to the continuing need for novel ways to treat cancers such as NSCLC, in particular SQLC, a reliable method to distinguish fibroblast growth factor receptor-inhibitor therapy responders from non-responders is urgently needed. Thus, the technical problem underlying the present invention is the provision of means and methods for the evaluation of subjects and/or cells for their responsiveness to anti-cancer treatment with inhibitors of the fibroblast growth factor receptor.

The technical problem is solved by provision of the embodiments characterized in the claims.

The present invention solves the above identified technical problem since, as documented herein below and in the appended examples, it was surprisingly found that a Breakage-Fusion-Bridge-like (BFB-like) mechanism can occur within the 8p arm. Interestingly, the BFB-like mechanism induces several head-to-head and tail-to-tail breaks within the 8p arm. Tail-to tail breaks close to the FGFR1 transcription start site induce focal FGFR1 amplification, deregulate FGFR1 transcription and are associated with FGFR inhibitor sensitivity. Furthermore, tail-to-tail breaks within FGFR1 Exon 1 to Exon 9 induce the deletion of the canonical FGFR1 ATG start codon, and the cancer cell uses a downstream in-frame ATG start codon. This often leads to transcription of a truncated FGFR1 version lagging the extracellular part of FGFR1, which has inhibitory function for FGFR1 signaling. Thus, these tail-to-tail intra-chromosomal breaks in FGFR1 and close to FGFR1 may work as a predictive therapeutic marker for an FGFR inhibitor therapy in cancer, such as NSCLC, in particular SQLC, that can be used to stratify patients for FGFR-inhibitor therapy. Analysis of the presence or absence of a genetic alteration of the FGFR1 gene, wherein the genetic alteration is a copy number variation caused by breaks located within, or within close proximity of, the ORF of the FGFR1 gene provides for the reliable prediction of the responsiveness of a tumor/cancer cell or an individual to cancer/tumor treatment with an FGFR1 inhibitor therapy.

### BRIEF DESCRIPTION OF THE INVENTION

Generally, and by way of brief description, the main aspects of the present invention can be described as follows:

In a first aspect, the invention pertains to a method for identifying a subject suffering from cancer as a responder or non-responder to fibroblast growth factor receptor 1 (FGFR1) inhibitor therapy.

In a second aspect, the invention pertains to an inhibitor of FGFR1 for use in the treatment of cancer in a patient.

In a third aspect, the invention pertains to a method for identifying a subject suffering from cancer as a responder or non-responder to a therapy with a receptor binding agent.

In a fourth aspect, the invention pertains to a method for identifying a genetic biomarker associated with a disorder.

In a fifth aspect, the invention pertains to a method for the diagnosis, prognosis, stratification and/or monitoring of a therapy, of cancer in a subject.

### DETAILED DESCRIPTION OF THE INVENTION

In the following, the elements of the invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine two or more of the explicitly described embodiments or which combine the one or more of the explicitly described embodiments with any number of the disclosed and/or preferred elements. Furthermore, any permutations and combinations of all described elements in this application should be considered disclosed by the description of the present application unless the context indicates otherwise.

In the first aspect, the invention pertains to a method for identifying a subject suffering from cancer as a responder or non-responder to fibroblast growth factor receptor 1 (FGFR1) inhibitor therapy, the method comprising the steps of
a) Providing from the subject a biological sample comprising genetic material associated with the cancer,
b) Determining in the genetic material the presence or absence of a genetic alteration of the FGFR1 gene, wherein the genetic alteration is a copy number variation caused by chromosomal breaks, such as a Breakage-Fusion-Bridge-like (BFB-like) mechanism, wherein the break is located within, or within close proximity of, the ORF of the FGFR1 gene;
   wherein the presence of the genetic alteration in the FGFR1 gene in the biological sample of the subject indicates that the subject is a responder to FGFR1-inhibitor therapy.

As used herein, the term “subject” or “patient” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject. In a particularly preferred embodiment, the subject is a mammal, such as a mouse, a rat, a guinea pig, a rabbit, a cat, a dog, a monkey, or a human, preferably a human, such as a human patient, more preferably a human patient at risk of suffering from cancer, and most preferably a human patient having received a lung transplantation, a lung allograft, a graft of hematopoetic stem cells, a chemotherapy, and/or a radiotherapy, optionally wherein said human patient is at risk of suffering from cancer.

As used herein, the term “subject at risk of suffering from cancer” or “human patient at risk of suffering from cancer” refers to a subject that presents one or more symptoms indicative of cancer.

Methods according to the present invention are useful for identifying a subject suffering from cancer as a responder or non-responder to fibroblast growth factor receptor 1 (FGFR1) inhibitor therapy, wherein the cancer is any kind of cancer. In a particularly preferred embodiment, the cancer is a solid cancer, preferably a cancer characterized by the expression of FGFR1, such as breast cancer, a gastric cancer, bladder cancer or lung cancer, and most preferably is Non-small cell lung cancer (NSCLC), such as squamous cell lung cancer (SQLC).

In a preferred embodiment, the method for identifying a subject suffering from cancer as a responder or non-responder to fibroblast growth factor receptor 1 (FGFR1) inhibitor therapy is an in vitro or ex vivo method. Since the herein described methods are non-invasive, the term “providing a biological” sample shall preferably not be interpreted to include a surgical procedure conducted at the subject. In a preferred embodiment, the subject is a human, preferably a human patient diagnosed with NSCLC, such as SQLC.

A “diagnosis” or the term “diagnostic” in context of the present invention means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

The term “biological sample” as used herein refers to a sample that was obtained and may be assayed for the presence or absence of a genetic alteration of the FGFR1 gene, wherein the genetic alteration is a copy number variation caused by chromosomal breaks, such as a Breakage-Fusion-Bridge-like (BFB-like) mechanism wherein the break is located within, or within close proximity of, the ORF of the FGFR1 gene as disclosed with the present invention. The biological sample can include a biological fluid (e.g., blood, cerebrospinal fluid, urine, plasma, serum), tissue biopsy, and the like. Preferably, the biological sample comprises tumour cells of the subject, preferably wherein the biological sample is a tumour biopsy, or a sample of a resected primary tumour.

To understand the underlying mechanism of FGFR1 dependency, the inventors performed whole genome and transcriptome sequencing of FGFR1-amplified primary tumors with unknown response upon FGFR inhibition. In addition, the inventors performed deep sequencing of FGFR1-amplified samples whereof the response upon FGFR inhibition was known for some of the samples (see Example section). In both cohorts the inventors identified intra-chromosomal tail-to-tail breaks close to the FGFR1 transcription start site, being responsible for focal amplification of FGFR1. In course of the present invention it was surprisingly found that these specific breaks are preferably caused by a Breakage-Fusion-Bridge-like (BFB-like) mechanism in squamous cell lung cancer. The amplification mechanism induces head-to-head followed by tail-to-tail intra-chromosomal breaks within the 8p arm.

Based on this mechanism the inventors were able to reconstruct the observed 8p11-p12 copy number profile, taking only all genomic breaks within the 8p-arm into account. Further, the inventors were able to show that tail-to-tail breaks close to the transcription start side of FGFR1 occurred in a multitude of 8p11-p12 amplified samples and led to focal amplification of FGFR1. This was primarily observed in cell lines, PDX models and patient samples responding upon an FGFR inhibitor therapy. In addition, the inventors could also show that in some FGFR1-amplified samples the break occurred within the open reading frame (ORF) of FGFR1, causing expression of a somatically altered variant of FGFR1 lacking the ectodomain (ΔEC-FGFR1). Specifically, the inventors observed the expression of short variants of FGFR1, and in particular to an altered FGFR1 transcript that lacks the oncogenic FGFR1 using non-canonical in frame ATG start codons, as shown by IHC staining, transcriptomic sequencing, and functional genomics.

Overexpression of the altered FGFR1 transcript transforms Baf3 cells and lead to an FGFR1-dependent phenotype. The inventors' results demonstrate that the truncation of the FGFR1 ectodomain is a frequent event in 8p11.23-amplified squamous cell lung cancer caused by tail-to-tail breaks. These breaks associate with FGFR inhibitor sensitivity and intra-chromosomal tail-to-tail breaks in FGFR1 may work as a predictive therapeutic marker for an FGFR inhibitor therapy in squamous cell lung cancer that can be used to stratify patients for FGFR-inhibitor therapy.

Accordingly, the methods and means of the present invention can be used to stratify patients into responders and non-responders to FGFR-inhibitor therapy. The term “stratify” or “stratification” for the purposes of this invention refers to the advantage that the method according to the invention renders possible decisions for the treatment and therapy of the patient, whether it is the hospitalization of the patient, the use, effect and/or dosage of one or more drugs, a therapeutic measure or the monitoring of a course of the disease and the course of therapy or etiology or classification of a disease, e.g., into a new or existing subtype or the differentiation of diseases and the patients thereof.

The methods and means of the present invention can also be used to monitor a therapy of cancer, in particular an FGFR-inhibitor therapy in a cancer patient. The term “monitoring a therapy” means for the purpose of the present invention to observe disease progression in a subject who receives a therapy. In other words, the subject during the therapy is regularly monitored for the effect of the applied therapy, which allows the medical practitioner to estimate at an early stage during the therapy whether the prescribed treatment is effective or not, and therefore to adjust the treatment regime accordingly.

As used herein the term “responsiveness to the inhibitor” or “responsive individual to the inhibitor” means in the context of the present invention that a mammalian tumor cell (or mammalian tumor) or mammalian cancer cell (or mammalian cancer) or an individual suffering from cancer, suspected to suffer from or being prone to suffer from cancer shows a response to an inhibitor of FGFR1 (or treatment with an inhibitor of FGFR1). A person of skill will readily be in the position to confirm that (a) cell(s) or individual(s) predicted to be responsive to an inhibitor of FGFR1 shows indeed a response to the inhibitor of FGFR1 (or to treatment with an inhibitor of FGFR1). For example, a response to an inhibitor may be reflected in a decreased suffering from cancer, such as a diminished and/or halted growth of a tumor and/or a reduction of the size of a tumor, the prevention of the formation of metastases or a reduction of number or size of metastases.

The term “response” as used herein may be reflected in form of a “complete response” or “partial response” of the herein identified individuals/patients or tumor cells. Such a “complete response” or “partial response” can primarily be seen in tumor cells or individuals with genetic modification of the FGFR1 locus resulting in a truncation of at least a part of an extracellular domain of FGFR1, and/or at targeted genetic copy number gain of a FGFR1 gene, or a modified variant thereof; and/or a consecutive palindromic arrangement of the FGFR1 gene, or a partial FGFR1 gene. Accordingly, a “complete response” or “partial response”, in particular a “complete response”, is preferred.

In this context, the term “response” can refer to a “tumor shrinkage”. “Tumor shrinkage” can refer to a reduced volume of the tumor upon treatment with the FGFR1 inhibitor compared to the initial volume at the start of (i.e. prior to) the treatment with the FGFR1 inhibitor. If the tumor volume is, for example, less than about 65% of the initial volume, the tumor has shrunk upon treatment. A tumor volume of, for example, less than 65% (like of from about 1% to 64%) compared to the initial tumor volume (100%) at the start of (i.e. prior to) the treatment with the FGFR1 inhibitor can represent a “partial response” (or partial remission; e.g. a decrease in the size of a tumor, or in the extent of cancer in the body, in response to treatment with the FGFR1 inhibitor). A “partial response” may encompass a (temporarily) tumor shrinkage/reduction in tumor volume during the course of the treatment compared to the initial tumor volume at the start of the treatment (i.e. prior to treatment).

Preferably, the patient group suffering from cancer and identified as a responder or non-responder to fibroblast growth factor receptor 1 (FGFR1) inhibitor therapy according to the present invention shows a “complete response”. The term “complete response” as used herein can refer to the disappearance of all signs of cancer in response to the inhibitor of FGFR1 (or treatment with an inhibitor of FGFR1). The term “complete response” and the term “complete remission” can be used interchangeably herein. For example, a “complete response” may be reflected in the continued shrinkage of the tumor until the tumor has disappeared. A tumor volume of, for example, 0% compared to the initial tumor volume (100%) at the start of (i.e. prior to) the treatment with the FGFR1 inhibitor can represent a “complete response”. Thus, the terms “responsiveness to the inhibitor” or “responsive individual to the inhibitor” as used herein may indicate the “complete response”/“complete responsiveness” of the tumor (cells), cancer (cells) or individuals predicted to respond in accordance with the present invention.

Any and all methods and means for identifying a subject suffering from cancer as a responder or non-responder to fibroblast growth factor receptor 1 (FGFR1) inhibitor therapy of this invention can be supplemented by conventional histological analysis of sampled specimens, which would allow an earlier and more specific diagnosis of cancer, making earlier treatment with potentially better outcome possible.

The term “FGFR1 inhibitor” or “inhibitor of FGFR1” or “antagonist of FGFR1” or any similar expressions shall in context of the present invention encompass any compound that has an activity as a modulator of the expression, function and/or stability of FGFR1, or of a variant of FGFR1. In some embodiments, the FGFR1 inhibitor can be selected from a small molecule, a polypeptide, peptide, glycoprotein, a peptide-mimetic, an antigen binding protein (ABP) (for example, an antibody, antibody-like molecule or other antigen binding derivative, or an or antigen binding fragment thereof), a nucleic acid such as a DNA or RNA, for example an antisense or inhibitory DNA or RNA, a ribozyme, an RNA or DNA aptamer, RNAi, siRNA, shRNA and the like, including variants or derivatives thereof such as a peptide nucleic acid (PNA), a genetic construct for targeted gene editing, such as a CRISPR/Cas9 construct and/or a guide nucleic acid (gRNA or gDNA) and/or tracrRNA.

In a preferred embodiment, the FGFR1 inhibitor is a selective or unselective FGFR1 inhibitor, such as an inhibitor of FGFR1, FGFR2, FGFR3, and/or FGFR4, such as an inhibitor selected from the group consisting of NVP-BGJ398, AZD4547, TKI258, JNJ42756493, pemigatinib, lucitanib, Rogaratinib, Debio 1347, FIIN2, LY2874455, PRN1371 and ponatinib.

Another preferred embodiment relates to the method for identifying a subject suffering from cancer as a responder or non-responder to FGFR1 inhibitor therapy, wherein the genetic alteration is
a) a genetic modification of the FGFR1 locus resulting in a truncation of at least a part of an extracellular domain of FGFR1, and/or
b) a genetic copy number gain of a FGFR1 gene, or a modified variant thereof; and/or
c) a consecutive palindromic arrangement of the FGFR1 gene, or a partial FGFR1 gene; and/or
d) a genetic modification of the FGFR1 locus resulting in the translation initiation of a non-canonical ATG start-codon.

A variant of FGFR1 is, in some embodiments, a protein comprising an amino acid sequence having at least 60%, 70%, 80%, 90%, preferably at least 80% such as at least 90%, sequence identity to SEQ ID NO: 1 (the human FGFR1 amino acid sequence), and most preferably at least 95% (such as at least 98%) sequence identity to SEQ ID NO: 1. In one preferred embodiment of the invention, the variant of FGFR1 comprises an amino acid sequence with at least 80% sequence identity to the amino acid sequence shown in SEQ ID NO: 1.

As used herein, the terms “identical” or percent “identity”, when used anywhere herein in the context of two or more nucleic acid or protein/polypeptide sequences, refer to two or more sequences or subsequences that are the same or have (or have at least) a specified percentage of amino acid residues or nucleotides that are the same (i.e., at, or at least, about 60% identity, preferably at, or at least, 65%, 70%, 75%, 80%, 85%, 90%, 91%, 92%, 93% or 94%, identity, and more preferably at, or at least, about 95%, 96%, 97%, 98%, 99%, or higher identity over a specified region-preferably over their full length sequences-, when compared and aligned for maximum correspondence over the comparison window or designated region) as measured using a sequence comparison algorithms, or by manual alignment and visual inspection (see, e.g., NCBI web site).

In a particularly preferred embodiment, the genetic alteration is an alteration of the FGFR1 gene resulting in the expression of an ectodomain-deleted variant of FGFR1, preferably wherein the variant of FGFR1 is translated from a non-canonical ATG in exon 1 to exon 9 of FGFR1.

A further aspect of the present invention pertains to an inhibitor of FGFR1 for use in the treatment of cancer in a patient, wherein the cancer is characterized by a genome which comprises a genetic alteration in the FGFR1 gene comprising a consecutive palindromic copy number variation of the FGFR1 gene, or of a fragment of the FGFR1 gene. Such a genetic alteration in the FGFR1 gene comprising a consecutive palindromic copy number variation of the FGFR1 gene, or of a fragment of the FGFR1 gene, can be the cause of a deregulated transcription.

A preferred embodiment relates to the inhibitor of FGFR1 for use in the treatment of cancer in a patient, wherein the consecutive palindromic copy number variation is an extraverted palindrome.

A further embodiment pertains to the inhibitor of FGFR1 for use in the treatment of cancer in a patient, wherein the inhibitor of FGFR1 is a small molecular inhibitor, an antigen binding protein, such as an antibody, or any antibody derivative, preferably which specifically binds to and inhibits FGFR, preferably FGFR1.

In a particularly preferred embodiment, the inhibitor of FGFR1 is a selective or unselective inhibitor of FGFR1, FGFR2, FGFR3, and/or FGFR4, preferably of FGFR1, such as an inhibitor selected from the group consisting of NVP-BG.J398, AZD4547, TKI258, JNJ42756493, pemigatinib, lucitanib, Rogaratinib, Debio 1347, FIIN2, LY2874455, PRN1371 and ponatinib.

The inhibitor of FGFR1 for use in the treatment of cancer in a patient according to the present invention is useful for the treatment of any kind of cancer. In a preferred embodiment, the inhibitor of FGFR1 is for use in the treatment of cancer in a patient, wherein the cancer is a solid cancer, preferably a cancer characterized by the expression of FGFR1, such as breast cancer, a gastric cancer, bladder cancer or lung cancer and most preferably is Non-small cell lung cancer (NSCLC), such as squamous cell lung cancer (SQLC).

Yet another preferred embodiment relates to the compound for use in the treatment and/or prevention of a proliferative disease in a subject, wherein the proliferative disease is a cancer which is refractory or resistant to treatment with at least one prior therapy.

In a particularly preferred embodiment, the genetic alteration is a focal amplification of the FGFR1 gene, or of a fragment of the FGFR1 gene.

A further embodiment pertains to the inhibitor of FGFR1 for use in the treatment of cancer in a patient, wherein the fragment of the FGFR1 gene is an expressible sequence and results in a FGFR1 protein variant, preferably an ectodomain-deleted FGFR1 protein variant.

Yet another embodiment relates to the inhibitor of FGFR1 for use in the treatment of cancer in a patient, wherein the treatment comprises a preliminary step of identifying the patient as a responder to an FGFR1 inhibitor therapy, wherein the preliminary step comprises a method according to the first aspect of the present invention.

In another aspect, which can be combined with any of the other aspects and any of the particularly preferred embodiments, the present invention provides a pharmaceutical composition for use in the treatment and/or prevention of cancer as described before, together with a pharmaceutically acceptable carrier and/or excipient.

The pharmaceutical composition of the invention is formulated to be compatible with its intended route of administration. Examples of routes of administration include parenteral, e.g., intrathecal, intra-arterial, intravenous, intradermal, subcutaneous, oral, transdermal (topical), intracerebroventricular, intraparenchymal, intratumoral and transmucosal administration. Generally, a pharmaceutical composition of the invention shall comprise one or more compounds for inhibiting FGFR1, and at least one pharmaceutically acceptable carrier and/or excipient. The pharmaceutical compositions as described herein are particularly useful for use in the herein described methods for treating proliferative diseases.

Solutions or suspensions used for parenteral, intradermal, or subcutaneous application can include the following components: a sterile diluent such as water for injection, saline solution, fixed oils, polyethylene glycols, glycerine; propylene glycol or other synthetic solvents; anti-bacterial agents such as benzyl alcohol or methyl parabens; antioxidants, such as ascorbic acid or sodium bisulfate; chelating agents such as ethylenediaminetetraacetic acid; buffers such as acetates, citrates or phosphates and agents for the adjustment of tonicity such as sodium chloride or dextrose.

Yet a further aspect of the present invention pertains to the use of an inhibitor of FGFR1 for the manufacture of a medicament for the treatment and/or prevention of a proliferative disease.

The herein disclosed compounds and pharmaceutical compositions are in particular useful in a method of preventing or treating a subject suffering from cancer comprising the administration of a therapeutically effective amount of an inhibitor of FGFR1 to the subject. In yet another aspect of this invention, which can be combined with any one of the other aspects and/or embodiments of this invention, there is provided a method for the treatment and/or prevention of a cancer disease in a subject, the method comprising one or more steps of administering to the subject a therapeutically effective amount of an inhibitor of FGFR1.

Treatment or prevention in some embodiments comprises the administration of a therapeutically effective amount of an FGFR1 inhibitor, wherein said inhibitor is preferably administered to said subject by intravenous, vaginal, oral, intranasal, intrathecal, intra-arterial, intradermal, subcutaneous, transdermal (topical), intracerebroventricular, intraparenchymal, intratumoral, transmucosal, rectal, bronchial, and/or parenteral administration, or by any clinically/medically accepted method.

Additionally preferred is that said therapeutically effective amount of an inhibitor of FGFR1 is provided in form of a tablet, a pill, a capsule, a troche, a dragée, a powder, an aerosol spray, a nasal spray, a suppository, a gel, an ointment, a salve, a cream, and/or a solution.

As used herein, the term “therapeutically effective amount” means that amount of a compound or pharmaceutical composition that will elicit the biological or medical response of a tissue, system, animal or human subject that is being sought, for instance, by a researcher or clinician, in accordance with the herein disclosed invention. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder.

For the prevention or treatment of a disease according to this invention, the appropriate dosage of an inhibitor of FGFR1 (or a pharmaceutical composition comprised thereof) will depend on the type of cancer to be treated, the severity and course of the disease, whether the inhibitor of FGFR1 and/or pharmaceutical composition is administered for preventive or therapeutic purposes, previous therapy, the patient's clinical history, age, size/weight and response to the inhibitor of FGFR1, and the decision of the attending physician. The inhibitor of FGFR1 and/or pharmaceutical composition is suitably administered to the patient at one time or over a series of treatments. If such inhibitor of FGFR1 and/or pharmaceutical composition is administered over a series of treatments, the total number of administrations for a given course of treatment may consist of a total of about 2, 3, 4, 5, 6, 7, 8, 9, 10 or more than about 10 treatments. For example, a treatment may be given once every day (or 2, 3 or 4 times a day) for a week, a month or even several months. In certain embodiments, the course of treatment may continue indefinitely.

Another aspect of the present invention pertains to a method for identifying a subject suffering from cancer as a responder or non-responder to a therapy with a receptor binding agent, the method comprising the steps of
a) Providing from the subject a biological sample comprising genetic material associated with the cancer,
b) Determining in the genetic material the presence or absence of a genetic alteration of a gene encoding for a receptor expressed in a cell associated with the cancer, wherein the genetic alteration is
   i. a copy number gain of the gene encoding for the receptor; and/or
   ii. a variant receptor gene encoding for a variant receptor protein which comprises a truncated extracellular domain, or no extracellular domain, of the receptor;
      wherein the presence of the genetic alteration in the biological sample of the subject indicates that the subject is a responder to a therapy with a receptor binding agent.

A preferred embodiment relates to the method for identifying a subject suffering from cancer as a responder or non-responder to a therapy with a receptor binding agent, wherein the receptor binding agent binds specifically to the receptor affected by said genetic alteration, preferably wherein the receptor binding agent is an inhibitor of the receptor.

In another preferred embodiment, the genetic alteration is a copy number gain of the variant receptor gene encoding for the variant receptor protein.

A further aspect of the present invention relates to a method for identifying a genetic biomarker associated with a disorder, the method comprising the steps of:
a) Providing a multiplicity of genetic samples of a patient group known to suffer from the disorder, and a multiplicity of genetic samples of a control group known to not suffer from the disorder;
b) Determining for one or more candidate genetic alterations a significant increased chance of occurrence in the multiplicity of genetic samples of a patient group compared to the multiplicity of genetic samples of a control group, wherein the candidate genetic alteration is the result of a tail-to-tail break from a Breakage-Fusion-Bridge (BFB) mechanism or a normal chromosomal break, optionally wherein a focal amplification of a gene is induced, and preferably is a consecutive palindromic copy number change of a genomic sequence;
   wherein if the candidate genetic alteration significantly occurs within the multiplicity of genetic samples of a patient group compared to the multiplicity of genetic samples of a control group, the candidate genetic alteration is a biomarker associated with the disorder.

A “biomarker” or “marker” in the context of the present invention refers to a genetic alteration, which is differentially present in a sample taken from subjects having a certain condition as compared to a comparable sample taken from subjects who do not have said condition.

In one embodiment, the genetic sample is a biological sample comprising genomic DNA, preferably a liquid sample, for example derived from blood, plasma, serum, saliva, urine, or is a tissue sample, such as a sample of a tumor tissue affected by the disorder.

A further embodiment relates to the method for identifying a genetic biomarker associated with a disorder, wherein the disorder is a proliferative disorder, such as a cancer, preferably a liquid or solid cancer. In another preferred embodiment, the cancer is breast cancer, a gastric cancer, bladder cancer or lung cancer, and most preferably is Non-small cell lung cancer (NSCLC), such as squamous cell lung cancer (SQLC).

In a particularly preferred embodiment, the genetic alteration is a copy number gain.

In another embodiment, the genomic sequence comprises a complete or partial sequence of a protein coding gene.

Yet another embodiment pertains to the method for identifying a genetic biomarker associated with a disorder, wherein the partial sequence of the protein coding gene still allows for the expression of a protein from the truncated open reading frame or the palindromic genetic locus, preferably without using the canonical ATG start codon, for example by using a non-canonical ATG in exon 1 to exon 9 of FGFR1.

In a further embodiment, the chromosomal break, such as the break of the Breakage-Fusion-Bridge-like (BFB-like) mechanism is located within the Open Reading Frame (ORF) of the protein coding gene, and preferably is in close proximity to the transcriptional start site, wherein a close proximity is within 1 mb, preferably within 800 kb, more preferably within 400 kb from the transcriptional start site.

Yet another embodiment relates to the method for identifying a genetic biomarker associated with a disorder, wherein the partial sequence of the protein coding gene is an expressible sequence and results in a FGFR1 protein variant, preferably an ectodomain-deleted FGFR1 protein variant.

Yet another aspect of this invention, which can be combined with any of the other aspects and any of the particularly preferred embodiments, pertains to a kit comprising means for determining the presence or absence of a genetic alteration of the FGFR1 gene. Preferably the kit of the invention is a kit for performing a method in accordance with the present invention comprising means for determining the presence or absence of a genetic alteration of the FGFR1 gene, wherein the genetic alteration is a copy number variation caused by a chromosomal break, such as a Breakage-Fusion-Bridge-like (BFB-like) mechanism, wherein the break is located within, or within close proximity of, the ORF of the FGFR1 gene.

Optionally, the kit can further comprise instructions for suitable operational parameters in the form of a label or a separate insert, and/or a container for holding its ingredients. For example, the kit may have standard instructions informing a consumer how to wash the probe after a sample of plasma is contacted on the probe.

In a further aspect of this invention, which can be combined with any of the other aspects and any of the particularly preferred embodiments, the present invention provides a kit comprising:
i) a therapeutically effective amount of a compound for use according to this invention, or a pharmaceutical composition for use according to this invention,
ii) written instructions to apply said therapeutically effective amount of said compound for use, or said pharmaceutical composition for use; and
iii) optionally, a container holding said compound, or said pharmaceutical composition, and the written instructions.

Optionally, the kit can further comprise instructions for suitable operational parameters in the form of a label or a separate insert. For example, the kit may have standard instructions informing a consumer how to wash the probe after a sample of plasma is contacted on the probe. In either embodiment, the kit may optionally further comprise a standard or control information so that the test sample can be compared with the control information standard.

Yet a further aspect of the present invention pertains to a method for the diagnosis, prognosis, stratification and/or monitoring of a therapy, of cancer in a subject, comprising the steps of:
a) Providing a biological sample from the subject, and
b) Determining in the biological sample the presence or absence of a chromosomal break, such as a Breakage-Fusion-Bridge-like (BFB-like) mechanism, wherein the break is located within, or within close proximity of, the ORF of the FGFR1 gene, an alteration of the mRNA-transcript of the FGFR1 gene, an alteration of the FGFR1 protein, an altered level of an mRNA-transcript of the FGFR1 gene, and/or an altered level of the FGFR1 protein, preferably wherein the extracellular domain of FGFR1 is deleted,
   wherein the presence of the chromosomal break, the alteration of the mRNA-transcript of the FGFR1 gene, the alteration of the FGFR1 protein, a differential level of an mRNA-transcript of the FGFR1 gene, and/or a differential level of the FGFR1 protein as determined in step (b) compared to a healthy control or reference value is indicative for the presence of cancer in the subject.

In a preferred embodiment of the above diagnostic method, the chromosomal break is a tail-to-tail Breakage-Fusion-Bridge-like (BFB-like) mechanism, wherein the break is located within, or within close proximity of, the ORF of the FGFR1 gene.

In another preferred embodiment, the chromosomal break induces:
a. a genetic modification of the FGFR1 locus resulting in a truncation of at least a part of an extracellular domain of FGFR1, and/or
b. a focal amplification of FGFR1, or of a fragment of the FGFR1 gene; and/or
c. a consecutive palindromic arrangement of the FGFR1 gene, or a partial FGFR1 gene; and/or
d. a genetic modification of the FGFR1 locus resulting in the translation initiation of a non-canonical ATG start-codon.

Further preferred is that the presence of the genetic modification of the FGFR1 gene, and/or the alteration of the mRNA-transcript of the FGFR1 gene results in the translation of an N-terminally truncated FGFR1 polypeptide. The method therefore preferably comprises detecting N-terminally truncated FGFR1 in a biological sample from the subject. More preferably, the method comprises the detection of a truncated FGFR1 polypeptide, wherein at least one IG-like domain is missing. In a particularly preferred embodiment, the determining of step b) of the method for the diagnosis, prognosis, stratification and/or monitoring of a therapy, of cancer in a subject is performed by immunohistochemistry (IHC).

In yet another preferred embodiment, the truncated FGFR1 variant is detected in step b) of the method for the diagnosis, prognosis, stratification and/or monitoring of a therapy, of cancer in a subject by the RNA technology “Archer” (Democratizing precision oncology (archerdx.com) or (Archer FusionPlex Comprehensive Thyroid & Lung (CTL)|Diagnóstica Longwood (dlongwood.com)).

In yet another preferred embodiment, the genetic alteration, the alteration of the mRNA-transcript of the FGFR1 gene, or the alteration of the FGFR1 protein comprises a sequence according to any one of SEQ ID Nos: 2 to 37.

Another preferred embodiment relates to the above method for the diagnosis, prognosis, stratification and/or monitoring of a therapy, of cancer in a subject, wherein the genetic alteration, and/or the alteration of the mRNA-transcript of the FGFR1 gene comprises a nucleic acid sequence according to any one of SEQ ID Nos: 2, 3, 10, 11, 15, 16, 20, 21, 25, 28, 31, 34, or 37, and/or wherein the altered FGFR1 protein comprises an amino acid sequence according to any one of SEQ ID Nos: 9, 14, 19, 24, 26, 29, 32, or 35.

The above method for the diagnosis, prognosis, stratification and/or monitoring of a therapy, of cancer in a subject is useful for diagnosing, prognosing, stratifying and/or monitoring any kind of cancer. Preferably, the cancer is a solid cancer, more preferably a cancer characterized by the expression of FGFR1, such as breast cancer, a gastric cancer, bladder cancer or lung cancer, and most preferably is Non-small cell lung cancer (NSCLC), such as squamous cell lung cancer (SQLC).

The terms “of the [present] invention”, “in accordance with the invention”, “according to the invention” and the like, as used herein are intended to refer to all aspects and embodiments of the invention described and/or claimed herein.

As used herein, the term “comprising” is to be construed as encompassing both “including” and “consisting of”, both meanings being specifically intended, and hence individually disclosed embodiments in accordance with the present invention. Where used herein, “and/or” is to be taken as specific disclosure of each of the two specified features or components with or without the other. For example, “A and/or B” is to be taken as specific disclosure of each of (i) A, (ii) B and (iii) A and B, just as if each is set out individually herein. In the context of the present invention, the terms “about” and “approximately” denote an interval of accuracy that the person skilled in the art will understand to still ensure the technical effect of the feature in question. The term typically indicates deviation from the indicated numerical value by ±20%, ±15%, ±10%, and for example±5%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. Where an indefinite or definite article is used when referring to a singular noun, e.g. “a”, “an” or “the”, this includes a plural of that noun unless something else is specifically stated.

It is to be understood that application of the teachings of the present invention to a specific problem or environment, and the inclusion of variations of the present invention or additional features thereto (such as further aspects and embodiments), will be within the capabilities of one having ordinary skill in the art in light of the teachings contained herein.

Unless context dictates otherwise, the descriptions and definitions of the features set out above are not limited to any particular aspect or embodiment of the invention and apply equally to all aspects and embodiments which are described.

All references, patents, and publications cited herein are hereby incorporated by reference in their entirety.

### BRIEF DESCRIPTION OF THE FIGURES AND SEQUENCES

The figures show:

FIG. 1 shows copy number analysis of samples with unknown FGFR inhibitor response. a) Average copy number plotted for 25 primary squamous cell lung cancer tumors with 8p11-p12 amplification at chromosome 8p11-p12. Copy numbers were calculated from tumor/normal whole-genome sequencing data and plotted with ROBOCOP (40×average sequencing depth). Line number 2 indicates average copy number of 25 samples with unknown FGFR inhibitor response. Line number 1 indicates average copy number of 9 primary tumor samples with tail-to-tail breaks close to FGFR1 (400 kb range). Line number 3 indicates average copy number of 16 primary tumor samples without tail-to-tail breaks close to FGFR1 (>1 mb). Locations of genes are annotated with a right-pointing triangle (coded on positive strand) or a left-pointing triangle (coded on negative strand). FGFR1 location is highlighted, and location of tail-to-tail breaks close to FGFR1 is also highlighted (left graph). Amplification peak of 9 samples is zoomed (right graph). Location of FGFR1 and NSD3 together with detected head-to-head (medium grey arrows), normal (light grey arrow), and tail-to-tail (black arrow) breaks within the genes are indicated. b) Domain architectures of NSD3 short (top, 1-645 AA) and NSD3 long (bottom, 1-1437 AA). Medium grey arrows indicate head-to-head, light grey arrows indicate normal breaks. N-terminus is right, C-terminus is left. c) Domain architecture of FGFR1 alpha (three IG domains). Detected tail-to-tail break is indicated (black arrow). TM=transmembrane domain, AB=acid box (self-inhibitory function), N-terminus (right) to C-terminus (left, 1-822 AA) d) Schematic overview of two random occurring breakage-fusion-bridge mechanisms forming 8p11-p12 amplifications. The two examples differ only in the consecutive order of breaks (indicated by arrows and I, II, III, and IV) e) One primer nested PCR over a detected head-to-head break (palindromic) using tumor-, matching normal-DNA (top) and the corresponding control (bottom, actin primers and no template) run at once and on the same gel f) Copy number plots of one tumor/normal whole genome sequenced sample (60× sequencing depth). Estimated copy number plot calculated from read counts (top), integral copy number plot calculated from read counts corrected by purity estimation (middle), and a copy number plot generated only from detected breaks and their coverage taking a breakage-fusion-bridge (BFB) mechanism into account (bottom). g) Evolution of the BFB shown in Figure if (bottom). The 8p arm is indicated by letters starting with A from the centrosome. In total 7 breaks were detected. One normal break (indicated by medium grey arrow) and 6 Breakage-Fusion-Bridge breaks (indicated by the first bridge and black arrows).

FIG. 2 shows copy number analysis of samples with known FGFR inhibitor response. a) Average GI50 values of 8 cell lines screened against the FGFR-inhibitors BG.J398 and AZD4547. Bars are indicated by number (1) (GI50>1 μM) or number (2) (GI50>1 μM) b) Average copy number of 6 cell lines not sensitive upon FGFR inhibition (marked by number (1)) and 2 cell lines which are sensitive upon FGFR inhibition (marked by number (2)). Copy number was extracted from genomic sequencing or whole exome sequencing data and plotted to chromosome 8 (hg19) with ROBOCOP. Location of NSD3 and FGFR1 with grey-backed surface c) Average tumor growth reduction of 8 patient derived xenograft tumor models treated with BG.J398 vs. vehicle. All models were confirmed to be FGFR1 amplified by FGFR1 FISH. Each bar represents an experiment of at least six mice and 12 tumors. Bars are marked by number (1) (tumor reduction of <30%) or by number (2) (tumor reduction of >30%). d) Average copy number of 5 patient derived xenograft tumor models not benefitting from FGFR inhibition (marked by number (1)) and 3 patient derived xenograft tumor (PDX) models which benefit from FGFR inhibition (marked by number (2)). Copy number was extracted from genomic sequencing data and plotted to chromosome 8 (hg19) with ROBOCOP. e) Tumor volume change of patients with squamous cell lung cancer harboring FGFR1 amplification and treated with BG.J398. Tumor volume change was measured by RECIST criteria. 5 patients had a progressive disease upon FGFR inhibition (marked by number (1)) and 3 patients showed durable, partial response upon FGFR inhibition (marked by number (2)). TUM003 and TUM007 died during treatment and no sign of response. * labeled sample was an of label FGFR inhibitor treated patient where no RECIST data were available. f) Average copy number of 5 patients with progressive disease (marked by number (2)) and 4 patients with durable, partial response upon FGFR inhibition (marked by number (1), one patient was treated with an off label used FGFR inhibitor (GW786034 HCl). Copy number was extracted from hybrid capture based sequencing and plotted to chromosome 8 (hg19) with ROBOCOP. g) Hybrid-capture-based genomic sequencing (558× depth) copy number plot zoomed to the location of FGFR1 (middle). Tumor sample from a patient responding upon treatment with an off label used FGFR inhibitor (GW786034 HCl). Normal exon structure of FGFR1 with genomic breaks are shown (bottom). Break coverage (top) and location of breaks are indicated (arrows). Corresponding HE and pFGFR1 IHC stains are shown (right) h) Resulting rearrangement of the tail-to-tail break is shown (dashed lines). i) Hybrid-capture-based genomic sequencing (615×depth) copy number plot zoomed to the location of FGFR1 (middle). Tumor sample is from a patient responding upon treatment with an FGFR inhibitor (BG.J398). Normal exon structure of FGFR1 (bottom), location of a tail-to-tail break (arrows), and break coverage (top) is shown. j) Resulting rearrangement of the tail-to-tail break is shown (dashed lines).

FIG. 3 shows the identification of a novel oncogenic ectodomain deleted FGFR1 version. a) Copy number plot of a tumor/normal whole-genome-sequenced (30×coverage) primary squamous cell lung cancer tumor and plotted with ROBOCOP (middle). Located genes in this region (middle, bottom; positive strand, negative strand), break coverage (top), location of tail-to-tail breaks (arrows and black dashed lines) are shown. Normal exon structure of NSD3 and FGFR1 with the genomic breaks are indicated by an asterisk independent of the chromosomal position shown above (bottom). b) Resulting rearrangement of the t-to-t break (dashed lines, top) and spanning reads from whole transcriptome sequencing data (bottom) is shown. c) PCR from corresponding cDNA with two different primer pairs, spanning the t-to-t break and FGFR1 (left) and matched normal cDNA (right) d) Hybrid-capture-based genomic sequencing (468×depth) copy number plot zoomed to the location of FGFR1 (middle). Tumor sample is from a patient with unknown response upon FGFR inhibition. Normal exon structure of FGFR1 (bottom), location of a tail-to-tail break (arrows), and break coverage (top) is shown. e) The resulting rearrangement of the t-to-t break is shown (dashed lines). f) Break spanning reads detected by ARCHER sequencing technique are indicated. g) Immunoblotting of transformed Baf3 cells (Baf3 FGFR1 AEC and Baf3 EML4-ALK as control) vs. non-transformed Baf3 cells transduced with empty Vector (Baf3 e.V.), FGFR1 alpha (Baf3 FGFR1aplpa), and FGFR1 beta (Baf3 FGFR1beta). Baf3 e.V, FGFR1 alpha and beta are cultured with IL3. h) Screening of Baf3 e.V., FGFR1 beta, and ectodomain lacking FGFR1 (FGFR1 AEC, using an inframe ATG in exon 9, NM_015850) using increasing concentrations of the FGFR inhibitor BG.J398 or i) the FGFR inhibitor AZD4547 (measuring cellular ATP content after 96 hours). Baf3 e.V. and Baf3 FGFR1 beta was screened with IL3 and Baf3 FGFR1 AEC was screened under IL3 depleted conditions.

The sequences show:

SEQ ID NO. 1:
Amino acid sequence of human Fibroblast
growth factor receptor 1 (FGFR1)
Met Trp Ser Trp Lys Cys Leu Leu Phe Trp Ala
Val Leu Val Thr Ala Thr Leu Cys Thr Ala Arg
Pro Ser Pro Thr Leu Pro Glu Gln Ala Gln Pro
Trp Gly Ala Pro Val Glu Val Glu Ser Phe Leu
Val His Pro Gly Asp Leu Leu Gln Leu Arg Cys
Arg Leu Arg Asp Asp Val Gln Ser Ile Asn Trp
Leu Arg Asp Gly Val Gln Leu Ala Glu Ser Asn
Arg Thr Arg Ile Thr Gly Glu Glu Val Glu Val
Gln Asp Ser Val Pro Ala Asp Ser Gly Leu Tyr
Ala Cys Val Thr Ser Ser Pro Ser Gly Ser Asp
Thr Thr Tyr Phe Ser Val Asn Val Ser Asp Ala
Leu Pro Ser Ser Glu Asp Asp Asp Asp Asp Asp
Asp Ser Ser Ser Glu Glu Lys Glu Thr Asp Asn
Thr Lys Pro Asn Arg Met Pro Val Ala Pro Tyr
Trp Thr Ser Pro Glu Lys Met Glu Lys Lys Leu
His Ala Val Pro Ala Ala Lys Thr Val Lys Phe
Lys Cys Pro Ser Ser Gly Thr Pro Asn Pro Thr
Leu Arg Trp Leu Lys Asn Gly Lys Glu Phe Lys
Pro Asp His Arg Ile Gly Gly Tyr Lys Val Arg
Tyr Ala Thr Trp Ser Ile Ile Met Asp Ser Val
Val Pro Ser Asp Lys Gly Asn Tyr Thr Cys Ile
Val Glu Asn Glu Tyr Gly Ser Ile Asn His Thr
Tyr Gln Leu Asp Val Val Glu Arg Ser Pro His
Arg Pro Ile Leu Gln Ala Gly Leu Pro Ala Asn
Lys Thr Val Ala Leu Gly Ser Asn Val Glu Phe
Met Cys Lys Val Tyr Ser Asp Pro Gln Pro His
Ile Gln Trp Leu Lys His Ile Glu Val Asn Gly
Ser Lys Ile Gly Pro Asp Asn Leu Pro Tyr Val
Gln Ile Leu Lys Thr Ala Gly Val Asn Thr Thr
Asp Lys Glu Met Glu Val Leu His Leu Arg Asn
Val Ser Phe Glu Asp Ala Gly Glu Tyr Thr Cys
Leu Ala Gly Asn Ser Ile Gly Leu Ser His His
Ser Ala Trp Leu Thr Val Leu Glu Ala Leu Glu
Glu Arg Pro Ala Val Met Thr Ser Pro Leu Tyr
Leu Glu Ile Ile Ile Tyr Cys Thr Gly Ala Phe
Leu Ile Ser Cys Met Val Gly Ser Val Ile Val
Tyr Lys Met Lys Ser Gly Thr Lys Lys Ser Asp
Phe His Ser Gln Met Ala Val His Lys Leu Ala
Lys Ser Ile Pro Leu Arg Arg Gln Val Thr Val
Ser Ala Asp Ser Ser Ala Ser Met Asn Ser Gly
Val Leu Leu Val Arg Pro Ser Arg Leu Ser Ser
Ser Gly Thr Pro Met Leu Ala Gly Val Ser Glu
Tyr Glu Leu Pro Glu Asp Pro Arg Trp Glu Leu
Pro Arg Asp Arg Leu Val Leu Gly Lys Pro Leu
Gly Glu Gly Cys Phe Gly Gln Val Val Leu Ala
Glu Ala Ile Gly Leu Asp Lys Asp Lys Pro Asn
Arg Val Thr Lys Val Ala Val Lys Met Leu Lys
Ser Asp Ala Thr Glu Lys Asp Leu Ser Asp Leu
Ile Ser Glu Met Glu Met Met Lys Met Ile Gly
Lys His Lys Asn Ile Ile Asn Leu Leu Gly Ala
Cys Thr Gln Asp Gly Pro Leu Tyr Val Ile Val
Glu Tyr Ala Ser Lys Gly Asn Leu Arg Glu Tyr
Leu Gln Ala Arg Arg Pro Pro Gly Leu Glu Tyr
Cys Tyr Asn Pro Ser His Asn Pro Glu Glu Gln
Leu Ser Ser Lys Asp Leu Val Ser Cys Ala Tyr
Gln Val Ala Arg Gly Met Glu Tyr Leu Ala Ser
Lys Lys Cys Ile His Arg Asp Leu Ala Ala Arg
Asn Val Leu Val Thr Glu Asp Asn Val Met Lys
Ile Ala Asp Phe Gly Leu Ala Arg Asp Ile His
His Ile Asp Tyr Tyr Lys Lys Thr Thr Asn Gly
Arg Leu Pro Val Lys Trp Met Ala Pro Glu Ala
Leu Phe Asp Arg Ile Tyr Thr His Gln Ser Asp
Val Trp Ser Phe Gly Val Leu Leu Trp Glu Ile
Phe Thr Leu Gly Gly Ser Pro Tyr Pro Gly Val
Pro Val Glu Glu Leu Phe Lys Leu Leu Lys Glu
Gly His Arg Met Asp Lys Pro Ser Asn Cys Thr
Asn Glu Leu Tyr Met Met Met Arg Asp Cys Trp
His Ala Val Pro Ser Gln Arg Pro Thr Phe Lys
Gln Leu Val Glu Asp Leu Asp Arg Ile Val Ala
Leu Thr Ser Asn Gln Glu Tyr Leu Asp Leu Ser
Met Pro Leu Asp Gln Tyr Ser Pro Ser Phe Pro
Asp Thr Arg Ser Ser Thr Cys Ser Ser Gly Glu
Asp Ser Val Phe Ser His Glu Pro Leu Pro Glu
Glu Pro Cys Leu Pro Arg His Pro Ala Gln Leu
Ala Asn Gly Gly Leu Lys Arg Arg

According to the present invention, genetic alterations of the FGFR1 gene are the result of a chromosomal break, such as a tail-to-tail Breakage-Fusion-Bridge (BFB)-like mechanism. These can be detected on several levels, such as on mRNA level. In general, any in-frame non-canonical ATG start codon in exon 1 to exon 9 of FGFR1 can be used for transcription of altered FGFR1 after a chromosomal break, such as a Breakage-Fusion-Bridge-like (BFB-like) mechanism. As an example, SEQ ID No: 2 shows the result of a genetic alteration of the FGFR1 gene that was caused by the tail-to-tail Breakage-Fusion-Bridge (BFB) mechanism.

SEQ ID No: 2
GTGGGAGAGTGACACACAAAACACCCCGGGAAGCCGGAGGAGGCG
TGGAAAGGTGGGGCCCGGGAGGTAAAACTGGGATCGGCGCGTTCA
GTGAGGCTGATTAAAGAACAACGCGCAACCAGCGCCATACGGCGC
TCCTCAGGGTGACTCGGCTCTCTAGCTCTTAAGGGTTAGAAGCGA
CCCTCGCACGGTCCCCGGTGCCACCTGGAGCATCATAATGGACTC
TGTGGTGCCCTCTGACAAGGGCAACTACACCTGCATTGTGGAGAA
TGAGTACGGCAGCATCAACCACACATACCAGCTGGATGTCGTGGA
GCGGTCCCCTCACCGGCCCATCCTGCAAGCAGGGTTGCCCGCCAA
CAAAACAGTGGCCCTGGGTAGCAACGTGGAGTTCATGTGTAAGGT
GTACAGTGACCCGCAGCCGCACATCCAGTGGCTAAAGCACATCGA
GGTGAATGGGAGCAAGATTGGCCCAGACAACCTGCCTTATGTCCA
GATCTTGAAGACTGCTGGAGTTAATACCACCGACAAAGAGATGGA
GGTGCTTCACTTAAGAAATGTCTCCTTTGAGGACGCAGGGGAGTA
TACGTGCTTGGCGGGTAACTCTATCGGACTCTCCCATCACTCTGC
ATGGTTGACCGTTCTGGAAGCCCTGGAAGAGAGGCCGGCAGTGAT
GACCTCGCCCCTGTACCTGGAGATCATCATCTATTGCACAGGGGC
CTTCCTCATCTCCTGCATGGTGGGGTCGGTCATCGTCTACAAGAT
GAAGAGTGGTACCAAGAAGAGTGACTTCCACAGCCAGATGGCTGT
GCACAAGCTGGCCAAGAGCATCCCTCTGCGCAGACAGGTAACAGT
GTCTGCTGACTCCAGTGCATCCATGAACTCTGGGGTTCTTCTGGT
TCGGCCATCACGGCTCTCCTCCAGTGGGACTCCCATGCTAGCAGG
GGTCTCTGAGTATGAGCTTCCCGAAGACCCTCGCTGGGAGCTGCC
TCGGGACAGACTGGTCTTAGGCAAACCCCTGGGAGAGGGCTGCTT
TGGGCAGGTGGTGTTGGCAGAGGCTATCGGGCTGGACAAGGACAA
ACCCAACCGTGTGACCAAAGTGGCTGTGAAGATGTTGAAGTCGGA
CGCAACAGAGAAAGACTTGTCAGACCTGATCTCAGAAATGGAGAT
GATGAAGATGATCGGGAAGCATAAGAATATCATCAACCTGCTGGG
GGCCTGCACGCAGGATGGTCCCTTGTATGTCATCGTGGAGTATGC
CTCCAAGGGCAACCTGCGGGAGTACCTGCAGGCCCGGAGGCCCCC
AGGGCTGGAATACTGCTACAACCCCAGCCACAACCCAGAGGAGCA
GCTCTCCTCCAAGGACCTGGTGTCCTGCGCCTACCAGGTGGCCCG
AGGCATGGAGTATCTGGCCTCCAAGAAGTGCATACACCGAGACCT
GGCAGCCAGGAATGTCCTGGTGACAGAGGACAATGTGATGAAGAT
AGCAGACTTTGGCCTCGCACGGGACATTCACCACATCGACTACTA
TAAAAAGACAACCAACGGCCGACTGCCTGTGAAGTGGATGGCACC
CGAGGCATTATTTGACCGGATCTACACCCACCAGAGTGATGTGTG
GTCTTTCGGGGTGCTCCTGTGGGAGATCTTCACTCTGGGCGGCTC
CCCATACCCCGGTGTGCCTGTGGAGGAACTTTTCAAGCTGCTGAA
GGAGGGTCACCGCATGGACAAGCCCAGTAACTGCACCAACGAGCT
GTACATGATGATGCGGGACTGCTGGCATGCAGTGCCCTCACAGAG
ACCCACCTTCAAGCAGCTGGTGGAAGACCTGGACCGCATCGTGGC
CTTGACCTCCAACCAGGAGTACCTGGACCTGTCCATGCCCCTGGA
CCAGTACTCCCCCAGCTTTCCCGACACCCGGAGCTCTACGTGCTC
CTCAGGGGAGGATTCCGTCTTCTCTCATGAGCCGCTGCCCGAGGA
GCCCTGCCTGCCCCGACACCCAGCCCAGCTTGCCAATGGCGGACT
CAAACGCCGCTGACTGCCACCCACACGCCCTCCCCAGACTCCACC
GTCAGCTGTAACCCTCACCCACAGCCCCTGCTGGGCCCACCACCT
GTCCGTCCCTGTCCCCTTTCCTGCTGGCAGGAGCCGGCTGCCTAC
CAGGGGCCTTCCTGTGTGGCCTGCCTTCACCCCACTCAGCTCACC
TCTCCCTCCACCTCCTCTCCACCTGCTGGTGAGAGGTGCAAAGAG
GCAGATCTTTGCTGCCAGCCACTTCATCCCCTCCCAGATGTTGGA
CCAACACCCCTCCCTGCCACCAGGCACTGCCTGGAGGGCAGGGAG
TGGGAGCCAATGAACAGGCATGCAAGTGAGAGCTTCCTGAGCTTT
CTCCTGTCGGTTTGGTCTGTTTTGCCTTCACCCATAAGCCCCTCG
CACTCTGGTGGCAGGTGCCTTGTCCTCAGGGCTACAGCAGTAGGG
AGGTCAGTGCTTCGTGCCTCGATTGAAGGTGACCTCTGCCCCAGA
TAGGTGGTGCCAGTGGCTTATTAATTCCGATACTAGTTTGCTTTG
CTGACCAAATGCCTGGTACCAGAGGATGGTGAGGCGAAGGCCAGG
TTGGGGGCAGTGTTGTGGCCCTGGGGCCCAGCCCCAAACTGGGGG
CTCTGTATATAGCTATGAAGAAAACACAAAGTGTATAAATCTGAG
TATATATTTACATGTCTTTTTAAAAGGGTCGTTACCAGAGATTTA
CCCATCGGGTAAGATGCTCCTGGTGGCTGGGAGGCATCAGTTGCT
ATATATTAAAAACAAAAAAGAAAAAAAAGGAAAATGTTTTTAAAA
AGGTCATATATTTTTTGCTACTTTTGCTGTTTTATTTTTTTAAAT
TATGTTCTAAACCTATTTTCAGTTTAGGTCCCTCAATAAAAATTG
CTGCTGCTTCA

The underlined part in SEQ ID No: 2 is being transcribed and is shown as SEQ ID No: 3 below.

SEQ ID No: 3
ATGGACTCTGTGGTGCCCTCTGACAAGGGCAACTACACCTGCATT
GTGGAGAATGAGTACGGCAGCATCAACCACACATACCAGCTGGAT
GTCGTGGAGCGGTCCCCTCACCGGCCCATCCTGCAAGCAGGGTTG
CCCGCCAACAAAACAGTGGCCCTGGGTAGCAACGTGGAGTTCATG
TGTAAGGTGTACAGTGACCCGCAGCCGCACATCCAGTGGCTAAAG
CACATCGAGGTGAATGGGAGCAAGATTGGCCCAGACAACCTGCCT
TATGTCCAGATCTTGAAGACTGCTGGAGTTAATACCACCGACAAA
GAGATGGAGGTGCTTCACTTAAGAAATGTCTCCTTTGAGGACGCA
GGGGAGTATACGTGCTTGGCGGGTAACTCTATCGGACTCTCCCAT
CACTCTGCATGGTTGACCGTTCTGGAAGCCCTGGAAGAGAGGCCG
GCAGTGATGACCTCGCCCCTGTACCTGGAGATCATCATCTATTGC
ACAGGGGCCTTCCTCATCTCCTGCATGGTGGGGTCGGTCATCGTC
TACAAGATGAAGAGTGGTACCAAGAAGAGTGACTTCCACAGCCAG
ATGGCTGTGCACAAGCTGGCCAAGAGCATCCCTCTGCGCAGACAG
GTAACAGTGTCTGCTGACTCCAGTGCATCCATGAACTCTGGGGTT
CTTCTGGTTCGGCCATCACGGCTCTCCTCCAGTGGGACTCCCATG
CTAGCAGGGGTCTCTGAGTATGAGCTTCCCGAAGACCCTCGCTGG
GAGCTGCCTCGGGACAGACTGGTCTTAGGCAAACCCCTGGGAGAG
GGCTGCTTTGGGCAGGTGGTGTTGGCAGAGGCTATCGGGCTGGAC
AAGGACAAACCCAACCGTGTGACCAAAGTGGCTGTGAAGATGTTG
AAGTCGGACGCAACAGAGAAAGACTTGTCAGACCTGATCTCAGAA
ATGGAGATGATGAAGATGATCGGGAAGCATAAGAATATCATCAAC
CTGCTGGGGGCCTGCACGCAGGATGGTCCCTTGTATGTCATCGTG
GAGTATGCCTCCAAGGGCAACCTGCGGGAGTACCTGCAGGCCCGG
AGGCCCCCAGGGCTGGAATACTGCTACAACCCCAGCCACAACCCA
GAGGAGCAGCTCTCCTCCAAGGACCTGGTGTCCTGCGCCTACCAG
GTGGCCCGAGGCATGGAGTATCTGGCCTCCAAGAAGTGCATACAC
CGAGACCTGGCAGCCAGGAATGTCCTGGTGACAGAGGACAATGTG
ATGAAGATAGCAGACTTTGGCCTCGCACGGGACATTCACCACATC
GACTACTATAAAAAGACAACCAACGGCCGACTGCCTGTGAAGTGG
ATGGCACCCGAGGCATTATTTGACCGGATCTACACCCACCAGAGT
GATGTGTGGTCTTTCGGGGTGCTCCTGTGGGAGATCTTCACTCTG
GGCGGCTCCCCATACCCCGGTGTGCCTGTGGAGGAACTTTTCAAG
CTGCTGAAGGAGGGTCACCGCATGGACAAGCCCAGTAACTGCACC
AACGAGCTGTACATGATGATGCGGGACTGCTGGCATGCAGTGCCC
TCACAGAGACCCACCTTCAAGCAGCTGGTGGAAGACCTGGACCGC
ATCGTGGCCTTGACCTCCAACCAGGAGTACCTGGACCTGTCCATG
CCCCTGGACCAGTACTCCCCCAGCTTTCCCGACACCCGGAGCTCT
ACGTGCTCCTCAGGGGAGGATTCCGTCTTCTCTCATGAGCCGCTG
CCCGAGGAGCCCTGCCTGCCCCGACACCCAGCCCAGCTTGCCAAT
GGCGGACTCAAACGCCGCTGA

The primers used for cloning of SEQ ID Nos: 2 and 3 are shown as SEQ ID Nos: 4 to 8 below.

(212_attB1_Soo674)
SEQ ID No: 4:
CACCTGGAGCATCATAATGGA
(213_attB1_Soo674)
SEQ ID No: 5:
TGGAGCATCATAATGGACTCTG
(220_attB2_FGFR1)
SEQ ID No: 6:
AGTCAGCGGCGTTTGAGTC
(221_attB2_FGFR1)
SEQ ID No: 7:
GTGTGGGTGGCAGTCAGC
(222_attB2_FGFR1)
SEQ ID No: 8:
CAGTCAGCGGCGTTTGAGT

Translation of SEQ ID No: 3 generates a protein comprising the amino acid sequence of SEQ ID No: 9.

SEQ ID No: 9
MDSVVPSDKGNYTCIVENEYGSINHTYQLDVVERSPHRPILQAGL
PANKTVALGSNVEFMCKVYSDPQPHIQWLKHIEVNGSKIGPDNLP
YVQILKTAGVNTTDKEMEVLHLRNVSFEDAGEYTCLAGNSIGLSH
HSAWLTVLEALEERPAVMTSPLYLEIIIYCTGAFLISCMVGSVIV
YKMKSGTKKSDFHSQMAVHKLAKSIPLRRQVSADSSASMNSGVLL
VRPSRLSSSGTPMLAGVSEYELPEDPRWELPRDRLVLGKPLGEGC
FGQVVLAEAIGLDKDKPNRVTKVAVKMLKSDATEKDLSDLISEME
MMKMIGKHKNIINLLGACTQDGPLYVIVEYASKGNLREYLQARRP
PGLEYCYNPSHNPEEQLSSKDLVSCAYQVARGMEYLASKKCIHRD
LAARNVLVTEDNVMKIADFGLARDIHHIDYYKKTTNGRLPVKWMA
PEALFDRIYTHQSDVWSFGVLLWEIFTLGGSPYPGVPVEELFKLL
KEGHRMDKPSNCTNELYMMMRDCWHAVPSQRPTFKQLVEDLDRIV
ALTSNQEYLDLSMPLDQYSPSFPDTRSSTCSSGEDSVFSHEPLPE
EPCLPRHPAQLANGGLKRR

Of note, the altered FGFR1 protein comprising the amino acid sequence of SEQ ID No: 9 has 604 amino acids and due to the N-terminal truncation, the altered FGFR1 protein comprises one IG-like-domain and the protein kinase domain.

FGFR1 transcripts of exemplary primary tumor samples of subjects that are responders to FGFR1-inhibitor therapy are shown in exemplary SEQ ID Nos. below. In principle all variants below can be transcribed (without 5′ UTR). As an example, SEQ ID No: 10 shows the transcript of sample A921, where a tail-to-tail-break within the intron before exon 9 was observed.

SEQ ID No: 10
CCCTGGAAGAGAGGCCGGCAGTGATGACCTCGCCCCTGTACCTGG
AGATCATCATCTATTGCACAGGGGCCTTCCTCATCTCCTGCATGG
TGGGGTCGGTCATCGTCTACAAGATGAAGAGTGGTACCAAGAAGA
GTGACTTCCACAGCCAGATGGCTGTGCACAAGCTGGCCAAGAGCA
TCCCTCTGCGCAGACAGGTGTCTGCTGACTCCAGTGCATCCATGA
ACTCTGGGGTTCTTCTGGTTCGGCCATCACGGCTCTCCTCCAGTG
GGACTCCCATGCTAGCAGGGGTCTCTGAGTATGAGCTTCCCGAAG
ACCCTCGCTGGGAGCTGCCTCGGGACAGACTGGTCTTAGGCAAAC
CCCTGGGAGAGGGCTGCTTTGGGCAGGTGGTGTTGGCAGAGGCTA
TCGGGCTGGACAAGGACAAACCCAACCGTGTGACCAAAGTGGCTG
TGAAGATGTTGAAGTCGGACGCAACAGAGAAAGACTTGTCAGACC
TGATCTCAGAAATGGAGATGATGAAGATGATCGGGAAGCATAAGA
ATATCATCAACCTGCTGGGGGCCTGCACGCAGGATGGTCCCTTGT
ATGTCATCGTGGAGTATGCCTCCAAGGGCAACCTGCGGGAGTACC
TGCAGGCCCGGAGGCCCCCAGGGCTGGAATACTGCTACAACCCCA
GCCACAACCCAGAGGAGCAGCTCTCCTCCAAGGACCTGGTGTCCT
GCGCCTACCAGGTGGCCCGAGGCATGGAGTATCTGGCCTCCAAGA
AGTGCATACACCGAGACCTGGCAGCCAGGAATGTCCTGGTGACAG
AGGACAATGTGATGAAGATAGCAGACTTTGGCCTCGCACGGGACA
TTCACCACATCGACTACTATAAAAAGACAACCAACGGCCGACTGC
CTGTGAAGTGGATGGCACCCGAGGCATTATTTGACCGGATCTACA
CCCACCAGAGTGATGTGTGGTCTTTCGGGGTGCTCCTGTGGGAGA
TCTTCACTCTGGGCGGCTCCCCATACCCCGGTGTGCCTGTGGAGG
AACTTTTCAAGCTGCTGAAGGAGGGTCACCGCATGGACAAGCCCA
GTAACTGCACCAACGAGCTGTACATGATGATGCGGGACTGCTGGC
ATGCAGTGCCCTCACAGAGACCCACCTTCAAGCAGCTGGTGGAAG
ACCTGGACCGCATCGTGGCCTTGACCTCCAACCAGGAGTACCTGG
ACCTGTCCATGCCCCTGGACCAGTACTCCCCCAGCTTTCCCGACA
CCCGGAGCTCTACGTGCTCCTCAGGGGAGGATTCCGTCTTCTCTC
ATGAGCCGCTGCCCGAGGAGCCCTGCCTGCCCCGACACCCAGCCC
AGCTTGCCAATGGCGGACTCAAACGCCGCTGACTGCCACCCACAC
GCCCTCCCCAGACTCCACCGTCAGCTGTAACCCTCACCCACAGCC
CCTGCTGGGCCCACCACCTGTCCGTCCCTGTCCCCTTTCCTGCTG
GCAGGAGCCGGCTGCCTACCAGGGGCCTTCCTGTGTGGCCTGCCT
TCACCCCACTCAGCTCACCTCTCCCTCCACCTCCTCTCCACCTGC
TGGTGAGAGGTGCAAAGAGGCAGATCTTTGCTGCCAGCCACTTCA
TCCCCTCCCAGATGTTGGACCAACACCCCTCCCTGCCACCAGGCA
CTGCCTGGAGGGCAGGGAGTGGGAGCCAATGAACAGGCATGCAAG
TGAGAGCTTCCTGAGCTTTCTCCTGTCGGTTTGGTCTGTTTTGCC
TTCACCCATAAGCCCCTCGCACTCTGGTGGCAGGTGCCTTGTCCT
CAGGGCTACAGCAGTAGGGAGGTCAGTGCTTCGTGCCTCGATTGA
AGGTGACCTCTGCCCCAGATAGGTGGTGCCAGTGGCTTATTAATT
CCGATACTAGTTTGCTTTGCTGACCAAATGCCTGGTACCAGAGGA
TGGTGAGGCGAAGGCCAGGTTGGGGGCAGTGTTGTGGCCCTGGGG
CCCAGCCCCAAACTGGGGGCTCTGTATATAGCTATGAAGAAAACA
CAAAGTGTATAAATCTGAGTATATATTTACATGTCTTTTTAAAAG
GGTCGTTACCAGAGATTTACCCATCGGGTAAGATGCTCCTGGTGG
CTGGGAGGCATCAGTTGCTATATATTAAAAACAAAAAAGAAAAAA
AAGGAAAATGTTTTTAAAAAGGTCATATATTTTTTGCTACTTTTG
CTGTTTTATTTTTTTAAATTATGTTCTAAACCTATTTTCAGTTTA
GGTCCCTCAATAAAAATTGCTGCTGCTTCA

The underlined part in SEQ ID No: 10 is being transcribed and is shown as SEQ ID No: 11 below.

SEQ ID No: 11
ATGACCTCGCCCCTGTACCTGGAGATCATCATCTATTGCACAGGG
GCCTTCCTCATCTCCTGCATGGTGGGGTCGGTCATCGTCTACAAG
ATGAAGAGTGGTACCAAGAAGAGTGACTTCCACAGCCAGATGGCT
GTGCACAAGCTGGCCAAGAGCATCCCTCTGCGCAGACAGGTGTCT
GCTGACTCCAGTGCATCCATGAACTCTGGGGTTCTTCTGGTTCGG
CCATCACGGCTCTCCTCCAGTGGGACTCCCATGCTAGCAGGGGTC
TCTGAGTATGAGCTTCCCGAAGACCCTCGCTGGGAGCTGCCTCGG
GACAGACTGGTCTTAGGCAAACCCCTGGGAGAGGGCTGCTTTGGG
CAGGTGGTGTTGGCAGAGGCTATCGGGCTGGACAAGGACAAACCC
AACCGTGTGACCAAAGTGGCTGTGAAGATGTTGAAGTCGGACGCA
ACAGAGAAAGACTTGTCAGACCTGATCTCAGAAATGGAGATGATG
AAGATGATCGGGAAGCATAAGAATATCATCAACCTGCTGGGGGCC
TGCACGCAGGATGGTCCCTTGTATGTCATCGTGGAGTATGCCTCC
AAGGGCAACCTGCGGGAGTACCTGCAGGCCCGGAGGCCCCCAGGG
CTGGAATACTGCTACAACCCCAGCCACAACCCAGAGGAGCAGCTC
TCCTCCAAGGACCTGGTGTCCTGCGCCTACCAGGTGGCCCGAGGC
ATGGAGTATCTGGCCTCCAAGAAGTGCATACACCGAGACCTGGCA
GCCAGGAATGTCCTGGTGACAGAGGACAATGTGATGAAGATAGCA
GACTTTGGCCTCGCACGGGACATTCACCACATCGACTACTATAAA
AAGACAACCAACGGCCGACTGCCTGTGAAGTGGATGGCACCCGAG
GCATTATTTGACCGGATCTACACCCACCAGAGTGATGTGTGGTCT
TTCGGGGTGCTCCTGTGGGAGATCTTCACTCTGGGCGGCTCCCCA
TACCCCGGTGTGCCTGTGGAGGAACTTTTCAAGCTGCTGAAGGAG
GGTCACCGCATGGACAAGCCCAGTAACTGCACCAACGAGCTGTAC
ATGATGATGCGGGACTGCTGGCATGCAGTGCCCTCACAGAGACCC
ACCTTCAAGCAGCTGGTGGAAGACCTGGACCGCATCGTGGCCTTG
ACCTCCAACCAGGAGTACCTGGACCTGTCCATGCCCCTGGACCAG
TACTCCCCCAGCTTTCCCGACACCCGGAGCTCTACGTGCTCCTCA
GGGGAGGATTCCGTCTTCTCTCATGAGCCGCTGCCCGAGGAGCCC
TGCCTGCCCCGACACCCAGCCCAGCTTGCCAATGGCGGACTCAAA
CGCCGCTGA

The primers used for cloning of SEQ ID Nos: 10 and 11 are shown as SEQ ID Nos: 12 to 13 below.

(214_attB1_A921)
SEQ ID No: 12
CCGGCAGTGATGACCTCG
(215_attB1_A921)
SEQ ID No: 13
CAGTGATGACCTCGCCCC

Translation of SEQ ID No: 11 generates a protein comprising the amino acid sequence of SEQ ID No: 14.

SEQ ID No: 14
MTSPLYLEIIIYCTGAFLISCMVGSVIVYKMKSGTKKSDFHSQMA
VHKLAKSIPLRRQVSADSSASMNSGVLLVRPSRLSSSGTPMLAGV
SEYELPEDPRWELPRDRLVLGKPLGEGCFGQVVLAEAIGLDKDKP
NRVTKVAVKMLKSDATEKDLSDLISEMEMMKMIGKHKNIINLLGA
CTQDGPLYVIVEYASKGNLREYLQARRPPGLEYCYNPSHNPEEQL
SSKDLVSCAYQVARGMEYLASKKCIHRDLAARNVLVTEDNVMKIA
DFGLARDIHHIDYYKKTTNGRLPVKWMAPEALFDRIYTHQSDVWS
FGVLLWEIFTLGGSPYPGVPVEELFKLLKEGHRMDKPSNCTNELY
MMMRDCWHAVPSQRPTFKQLVEDLDRIVALTSNQEYLDLSMPLDQ
YSPSFPDTRSSTCSSGEDSVFSHEPLPEEPCLPRHPAQLANGGLK
RR

Of note, the altered FGFR1 protein comprising the amino acid sequence of SEQ ID No: 14 has 452 amino acids comprising no IG-like-domain due to the N-terminal truncation, but the protein kinase domain. In the context of the present invention, a polypeptide comprising the amino acid sequence of SEQ ID No: 14 is also referred to as ΔEC-FGFR1. The double-underlined residues in SEQ ID No: 14 form the transmembrane domain, whereas the single-underlined residues form the kinase domain.

SEQ ID No: 15 shows the transcript of sample p68.

SEQ ID No: 15
CCGTAGCTCCATATTGGACATCCCCAGAAAAGATGGAAAAGAAAT
TGCATGCAGTGCCGGCTGCCAAGACAGTGAAGTTCAAATGCCCTT
CCAGTGGGACCCCAAACCCCACACTGCGCTGGTTGAAAAATGGCA
AAGAATTCAAACCTGACCACAGAATTGGAGGCTACAAGGTCCGTT
ATGCCACCTGGAGCATCATAATGGACTCTGTGGTGCCCTCTGACA
AGGGCAACTACACCTGCATTGTGGAGAATGAGTACGGCAGCATCA
ACCACACATACCAGCTGGATGTCGTGGAGCGGTCCCCTCACCGGC
CCATCCTGCAAGCAGGGTTGCCCGCCAACAAAACAGTGGCCCTGG
GTAGCAACGTGGAGTTCATGTGTAAGGTGTACAGTGACCCGCAGC
CGCACATCCAGTGGCTAAAGCACATCGAGGTGAATGGGAGCAAGA
TTGGCCCAGACAACCTGCCTTATGTCCAGATCTTGAAGACTGCTG
GAGTTAATACCACCGACAAAGAGATGGAGGTGCTTCACTTAAGAA
ATGTCTCCTTTGAGGACGCAGGGGAGTATACGTGCTTGGCGGGTA
ACTCTATCGGACTCTCCCATCACTCTGCATGGTTGACCGTTCTGG
AAGCCCTGGAAGAGAGGCCGGCAGTGATGACCTCGCCCCTGTACC
TGGAGATCATCATCTATTGCACAGGGGCCTTCCTCATCTCCTGCA
TGGTGGGGTCGGTCATCGTCTACAAGATGAAGAGTGGTACCAAGA
AGAGTGACTTCCACAGCCAGATGGCTGTGCACAAGCTGGCCAAGA
GCATCCCTCTGCGCAGACAGGTGTCTGCTGACTCCAGTGCATCCA
TGAACTCTGGGGTTCTTCTGGTTCGGCCATCACGGCTCTCCTCCA
GTGGGACTCCCATGCTAGCAGGGGTCTCTGAGTATGAGCTTCCCG
AAGACCCTCGCTGGGAGCTGCCTCGGGACAGACTGGTCTTAGGCA
AACCCCTGGGAGAGGGCTGCTTTGGGCAGGTGGTGTTGGCAGAGG
CTATCGGGCTGGACAAGGACAAACCCAACCGTGTGACCAAAGTGG
CTGTGAAGATGTTGAAGTCGGACGCAACAGAGAAAGACTTGTCAG
ACCTGATCTCAGAAATGGAGATGATGAAGATGATCGGGAAGCATA
AGAATATCATCAACCTGCTGGGGGCCTGCACGCAGGATGGTCCCT
TGTATGTCATCGTGGAGTATGCCTCCAAGGGCAACCTGCGGGAGT
ACCTGCAGGCCCGGAGGCCCCCAGGGCTGGAATACTGCTACAACC
CCAGCCACAACCCAGAGGAGCAGCTCTCCTCCAAGGACCTGGTGT
CCTGCGCCTACCAGGTGGCCCGAGGCATGGAGTATCTGGCCTCCA
AGAAGTGCATACACCGAGACCTGGCAGCCAGGAATGTCCTGGTGA
CAGAGGACAATGTGATGAAGATAGCAGACTTTGGCCTCGCACGGG
ACATTCACCACATCGACTACTATAAAAAGACAACCAACGGCCGAC
TGCCTGTGAAGTGGATGGCACCCGAGGCATTATTTGACCGGATCT
ACACCCACCAGAGTGATGTGTGGTCTTTCGGGGTGCTCCTGTGGG
AGATCTTCACTCTGGGCGGCTCCCCATACCCCGGTGTGCCTGTGG
AGGAACTTTTCAAGCTGCTGAAGGAGGGTCACCGCATGGACAAGC
CCAGTAACTGCACCAACGAGCTGTACATGATGATGCGGGACTGCT
GGCATGCAGTGCCCTCACAGAGACCCACCTTCAAGCAGCTGGTGG
AAGACCTGGACCGCATCGTGGCCTTGACCTCCAACCAGGAGTACC
TGGACCTGTCCATGCCCCTGGACCAGTACTCCCCCAGCTTTCCCG
ACACCCGGAGCTCTACGTGCTCCTCAGGGGAGGATTCCGTCTTCT
CTCATGAGCCGCTGCCCGAGGAGCCCTGCCTGCCCCGACACCCAG
CCCAGCTTGCCAATGGCGGACTCAAACGCCGCTGACTGCCACCCA
CACGCCCTCCCCAGACTCCACCGTCAGCTGTAACCCTCACCCACA
GCCCCTGCTGGGCCCACCACCTGTCCGTCCCTGTCCCCTTTCCTG
CTGGCAGGAGCCGGCTGCCTACCAGGGGCCTTCCTGTGTGGCCTG
CCTTCACCCCACTCAGCTCACCTCTCCCTCCACCTCCTCTCCACC
TGCTGGTGAGAGGTGCAAAGAGGCAGATCTTTGCTGCCAGCCACT
TCATCCCCTCCCAGATGTTGGACCAACACCCCTCCCTGCCACCAG
GCACTGCCTGGAGGGCAGGGAGTGGGAGCCAATGAACAGGCATGC
AAGTGAGAGCTTCCTGAGCTTTCTCCTGTCGGTTTGGTCTGTTTT
GCCTTCACCCATAAGCCCCTCGCACTCTGGTGGCAGGTGCCTTGT
CCTCAGGGCTACAGCAGTAGGGAGGTCAGTGCTTCGTGCCTCGAT
TGAAGGTGACCTCTGCCCCAGATAGGTGGTGCCAGTGGCTTATTA
ATTCCGATACTAGTTTGCTTTGCTGACCAAATGCCTGGTACCAGA
GGATGGTGAGGCGAAGGCCAGGTTGGGGGCAGTGTTGTGGCCCTG
GGGCCCAGCCCCAAACTGGGGGCTCTGTATATAGCTATGAAGAAA
ACACAAAGTGTATAAATCTGAGTATATATTTACATGTCTTTTTAA
AAGGGTCGTTACCAGAGATTTACCCATCGGGTAAGATGCTCCTGG
TGGCTGGGAGGCATCAGTTGCTATATATTAAAAACAAAAAAGAAA
AAAAAGGAAAATGTTTTTAAAAAGGTCATATATTTTTTGCTACTT
TTGCTGTTTTATTTTTTTAAATTATGTTCTAAACCTATTTTCAGT
TTAGGTCCCTCAATAAAAATTGCTGCTGCTTCA

The underlined part in SEQ ID No: 15 is being transcribed and is shown as SEQ ID No: 16 below.

SEQ ID No: 16
ATGGAAAAGAAATTGCATGCAGTGCCGGCTGCCAAGACAGTGAAGTTCAA
ATGCCCTTCCAGTGGGACCCCAAACCCCACACTGCGCTGGTTGAAAAATG
GCAAAGAATTCAAACCTGACCACAGAATTGGAGGCTACAAGGTCCGTTAT
GCCACCTGGAGCATCATAATGGACTCTGTGGTGCCCTCTGACAAGGGCAA
CTACACCTGCATTGTGGAGAATGAGTACGGCAGCATCAACCACACATACC
AGCTGGATGTCGTGGAGCGGTCCCCTCACCGGCCCATCCTGCAAGCAGGG
TTGCCCGCCAACAAAACAGTGGCCCTGGGTAGCAACGTGGAGTTCATGTG
TAAGGTGTACAGTGACCCGCAGCCGCACATCCAGTGGCTAAAGCACATCG
AGGTGAATGGGAGCAAGATTGGCCCAGACAACCTGCCTTATGTCCAGATC
TTGAAGACTGCTGGAGTTAATACCACCGACAAAGAGATGGAGGTGCTTCA
CTTAAGAAATGTCTCCTTTGAGGACGCAGGGGAGTATACGTGCTTGGCGG
GTAACTCTATCGGACTCTCCCATCACTCTGCATGGTTGACCGTTCTGGAA
GCCCTGGAAGAGAGGCCGGCAGTGATGACCTCGCCCCTGTACCTGGAGAT
CATCATCTATTGCACAGGGGCCTTCCTCATCTCCTGCATGGTGGGGTCGG
TCATCGTCTACAAGATGAAGAGTGGTACCAAGAAGAGTGACTTCCACAGC
CAGATGGCTGTGCACAAGCTGGCCAAGAGCATCCCTCTGCGCAGACAGGT
GTCTGCTGACTCCAGTGCATCCATGAACTCTGGGGTTCTTCTGGTTCGGC
CATCACGGCTCTCCTCCAGTGGGACTCCCATGCTAGCAGGGGTCTCTGAG
TATGAGCTTCCCGAAGACCCTCGCTGGGAGCTGCCTCGGGACAGACTGGT
CTTAGGCAAACCCCTGGGAGAGGGCTGCTTTGGGCAGGTGGTGTTGGCAG
AGGCTATCGGGCTGGACAAGGACAAACCCAACCGTGTGACCAAAGTGGCT
GTGAAGATGTTGAAGTCGGACGCAACAGAGAAAGACTTGTCAGACCTGAT
CTCAGAAATGGAGATGATGAAGATGATCGGGAAGCATAAGAATATCATCA
ACCTGCTGGGGGCCTGCACGCAGGATGGTCCCTTGTATGTCATCGTGGAG
TATGCCTCCAAGGGCAACCTGCGGGAGTACCTGCAGGCCCGGAGGCCCCC
AGGGCTGGAATACTGCTACAACCCCAGCCACAACCCAGAGGAGCAGCTCT
CCTCCAAGGACCTGGTGTCCTGCGCCTACCAGGTGGCCCGAGGCATGGAG
TATCTGGCCTCCAAGAAGTGCATACACCGAGACCTGGCAGCCAGGAATGT
CCTGGTGACAGAGGACAATGTGATGAAGATAGCAGACTTTGGCCTCGCAC
GGGACATTCACCACATCGACTACTATAAAAAGACAACCAACGGCCGACTG
CCTGTGAAGTGGATGGCACCCGAGGCATTATTTGACCGGATCTACACCCA
CCAGAGTGATGTGTGGTCTTTCGGGGTGCTCCTGTGGGAGATCTTCACTC
TGGGCGGCTCCCCATACCCCGGTGTGCCTGTGGAGGAACTTTTCAAGCTG
CTGAAGGAGGGTCACCGCATGGACAAGCCCAGTAACTGCACCAACGAGCT
GTACATGATGATGCGGGACTGCTGGCATGCAGTGCCCTCACAGAGACCCA
CCTTCAAGCAGCTGGTGGAAGACCTGGACCGCATCGTGGCCTTGACCTCC
AACCAGGAGTACCTGGACCTGTCCATGCCCCTGGACCAGTACTCCCCCAG
CTTTCCCGACACCCGGAGCTCTACGTGCTCCTCAGGGGAGGATTCCGTCT
TCTCTCATGAGCCGCTGCCCGAGGAGCCCTGCCTGCCCCGACACCCAGCC
CAGCTTGCCAATGGCGGACTCAAACGCCGCTGA

The primers used for cloning of SEQ ID Nos: 15 and 16 are shown as SEQ ID Nos: 17 to 18 below.

SEQ ID No: 17
TTGGACATCCCCAGAAAAGA (216_attB1_p68)
SEQ ID No: 18
CCATATTGGACATCCCCAGA (217_attB1_p68)

Translation of SEQ ID No: 16 generates a protein comprising the amino acid sequence of SEQ ID No: 19.

SEQ ID No: 19
MEKKLHAVPAAKTVKFKCPSSGTPNPTLRWLKNGKEFKPDHRIGGYKVRY
ATWSIIMDSVVPSDKGNYTCIVENEYGSINHTYQLDVVERSPHRPILQAG
LPANKTVALGSNVEFMCKVYSDPQPHIQWLKHIEVNGSKIGPDNLPYVQI
LKTAGVNTTDKEMEVLHLRNVSFEDAGEYTCLAGNSIGLSHHSAWLTVLE
ALEERPAVMTSPLYLEIIIYCTGAFLISCMVGSVIVYKMKSGTKKSDFHS
QMAVHKLAKSIPLRRQVSADSSASMNSGVLLVRPSRLSSSGTPMLAGVSE
YELPEDPRWELPRDRLVLGKPLGEGCFGQVVLAEAIGLDKDKPNRVTKVA
VKMLKSDATEKDLSDLISEMEMMKMIGKHKNIINLLGACTQDGPLYVIVE
YASKGNLREYLQARRPPGLEYCYNPSHNPEEQLSSKDLVSCAYQVARGME
YLASKKCIHRDLAARNVLVTEDNVMKIADFGLARDIHHIDYYKKTTNGRL
PVKWMAPEALFDRIYTHQSDVWSFGVLLWEIFTLGGSPYPGVPVEELFKL
LKEGHRMDKPSNCTNELYMMMRDCWHAVPSQRPTFKQLVEDLDRIVALTS
NQEYLDLSMPLDQYSPSFPDTRSSTCSSGEDSVFSHEPLPEEPCLPRHPA
QLANGGLKRR

The altered FGFR1 protein comprising the amino acid sequence of SEQ ID No: 19 has 660 amino acids comprising two IG-like-domains, and the protein kinase domain.

SEQ ID No: 20 shows the transcript of sample A1116. Of note, a break within the 5′ UTR (intron 1 of FGFR1) results in deletion of a multitude of regulatory elements on DNA level. On mRNA level, a deletion of the 5′ UTR (coded from exon 1 of FGFR1) can be observed. SEQ ID No: 20 comprises the ATG start codon of wt-FGFR1 coded in exon 2. Translation of the transcript with SEQ ID No: 20 corresponds to wt-FGFR1 (SEQ ID No: 1; FGFR1 NM_015850).

SEQ ID No: 20
ACTGCTGGAGTTAATACCACCGACAAAGAGATGGAGGTGCTTCACTTAAG
AAATGTCTCCTTTGAGGACGCAGGGGAGTATACGTGCTTGGCGGGTAACT
CTATCGGACTCTCCCATCACTCTGCATGGTTGACCGTTCTGGAAGCCCTG
GAAGAGAGGCCGGCAGTGATGACCTCGCCCCTGTACCTGGAGATCATCAT
CTATTGCACAGGGGCCTTCCTCATCTCCTGCATGGTGGGGTCGGTCATCG
TCTACAAGATGAAGAGTGGTACCAAGAAGAGTGACTTCCACAGCCAGATG
GCTGTGCACAAGCTGGCCAAGAGCATCCCTCTGCGCAGACAGGTAACAGT
GTCTGCTGACTCCAGTGCATCCATGAACTCTGGGGTTCTTCTGGTTCGGC
CATCACGGCTCTCCTCCAGTGGGACTCCCATGCTAGCAGGGGTCTCTGAG
TATGAGCTTCCCGAAGACCCTCGCTGGGAGCTGCCTCGGGACAGACTGGT
CTTAGGCAAACCCCTGGGAGAGGGCTGCTTTGGGCAGGTGGTGTTGGCAG
AGGCTATCGGGCTGGACAAGGACAAACCCAACCGTGTGACCAAAGTGGCT
GTGAAGATGTTGAAGTCGGACGCAACAGAGAAAGACTTGTCAGACCTGAT
CTCAGAAATGGAGATGATGAAGATGATCGGGAAGCATAAGAATATCATCA
ACCTGCTGGGGGCCTGCACGCAGGATGGTCCCTTGTATGTCATCGTGGAG
TATGCCTCCAAGGGCAACCTGCGGGAGTACCTGCAGGCCCGGAGGCCCCC
AGGGCTGGAATACTGCTACAACCCCAGCCACAACCCAGAGGAGCAGCTCT
CCTCCAAGGACCTGGTGTCCTGCGCCTACCAGGTGGCCCGAGGCATGGAG
TATCTGGCCTCCAAGAAGTGCATACACCGAGACCTGGCAGCCAGGAATGT
CCTGGTGACAGAGGACAATGTGATGAAGATAGCAGACTTTGGCCTCGCAC
GGGACATTCACCACATCGACTACTATAAAAAGACAACCAACGGCCGACTG
CCTGTGAAGTGGATGGCACCCGAGGCATTATTTGACCGGATCTACACCCA
CCAGAGTGATGTGTGGTCTTTCGGGGTGCTCCTGTGGGAGATCTTCACTC
TGGGCGGCTCCCCATACCCCGGTGTGCCTGTGGAGGAACTTTTCAAGCTG
CTGAAGGAGGGTCACCGCATGGACAAGCCCAGTAACTGCACCAACGAGCT
GTACATGATGATGCGGGACTGCTGGCATGCAGTGCCCTCACAGAGACCCA
CCTTCAAGCAGCTGGTGGAAGACCTGGACCGCATCGTGGCCTTGACCTCC
AACCAGGAGTACCTGGACCTGTCCATGCCCCTGGACCAGTACTCCCCCAG
CTTTCCCGACACCCGGAGCTCTACGTGCTCCTCAGGGGAGGATTCCGTCT
TCTCTCATGAGCCGCTGCCCGAGGAGCCCTGCCTGCCCCGACACCCAGCC
CAGCTTGCCAATGGCGGACTCAAACGCCGCTGACTGCCACCCACACGCCC
TCCCCAGACTCCACCGTCAGCTGTAACCCTCACCCACAGCCCCTGCTGGG
CCCACCACCTGTCCGTCCCTGTCCCCTTTCCTGCTGGCAGGAGCCGGCTG
CCTACCAGGGGCCTTCCTGTGTGGCCTGCCTTCACCCCACTCAGCTCACC
TCTCCCTCCACCTCCTCTCCACCTGCTGGTGAGAGGTGCAAAGAGGCAGA
TCTTTGCTGCCAGCCACTTCATCCCCTCCCAGATGTTGGACCAACACCCC
TCCCTGCCACCAGGCACTGCCTGGAGGGCAGGGAGTGGGAGCCAATGAAC
AGGCATGCAAGTGAGAGCTTCCTGAGCTTTCTCCTGTCGGTTTGGTCTGT
TTTGCCTTCACCCATAAGCCCCTCGCACTCTGGTGGCAGGTGCCTTGTCC
TCAGGGCTACAGCAGTAGGGAGGTCAGTGCTTCGTGCCTCGATTGAAGGT
GACCTCTGCCCCAGATAGGTGGTGCCAGTGGCTTATTAATTCCGATACTA
GTTTGCTTTGCTGACCAAATGCCTGGTACCAGAGGATGGTGAGGCGAAGG
CCAGGTTGGGGGCAGTGTTGTGGCCCTGGGGCCCAGCCCCAAACTGGGGG
CTCTGTATATAGCTATGAAGAAAACACAAAGTGTATAAATCTGAGTATAT
ATTTACATGTCTTTTTAAAAGGGTCGTTACCAGAGATTTACCCATCGGGT
AAGATGCTCCTGGTGGCTGGGAGGCATCAGTTGCTATATATTAAAAACAA
AAAAGAAAAAAAAGGAAAATGTTTTTAAAAAGGTCATATATTTTTTGCTA
CTTTTGCTGTTTTATTTTTTTAAATTATGTTCTAAACCTATTTTCAGTTT
AGGTCCCTCAATAAAAATTGCTGCTGCTTCA

The underlined part in SEQ ID No: 20 is being transcribed and is shown as SEQ ID No: 21 below.

SEQ ID No: 21
ATGGAGGTGCTTCACTTAAGAAATGTCTCCTTTGAGGACGCAGGGGAGTA
TACGTGCTTGGCGGGTAACTCTATCGGACTCTCCCATCACTCTGCATGGT
TGACCGTTCTGGAAGCCCTGGAAGAGAGGCCGGCAGTGATGACCTCGCCC
CTGTACCTGGAGATCATCATCTATTGCACAGGGGCCTTCCTCATCTCCTG
CATGGTGGGGTCGGTCATCGTCTACAAGATGAAGAGTGGTACCAAGAAGA
GTGACTTCCACAGCCAGATGGCTGTGCACAAGCTGGCCAAGAGCATCCCT
CTGCGCAGACAGGTAACAGTGTCTGCTGACTCCAGTGCATCCATGAACTC
TGGGGTTCTTCTGGTTCGGCCATCACGGCTCTCCTCCAGTGGGACTCCCA
TGCTAGCAGGGGTCTCTGAGTATGAGCTTCCCGAAGACCCTCGCTGGGAG
CTGCCTCGGGACAGACTGGTCTTAGGCAAACCCCTGGGAGAGGGCTGCTT
TGGGCAGGTGGTGTTGGCAGAGGCTATCGGGCTGGACAAGGACAAACCCA
ACCGTGTGACCAAAGTGGCTGTGAAGATGTTGAAGTCGGACGCAACAGAG
AAAGACTTGTCAGACCTGATCTCAGAAATGGAGATGATGAAGATGATCGG
GAAGCATAAGAATATCATCAACCTGCTGGGGGCCTGCACGCAGGATGGTC
CCTTGTATGTCATCGTGGAGTATGCCTCCAAGGGCAACCTGCGGGAGTAC
CTGCAGGCCCGGAGGCCCCCAGGGCTGGAATACTGCTACAACCCCAGCCA
CAACCCAGAGGAGCAGCTCTCCTCCAAGGACCTGGTGTCCTGCGCCTACC
AGGTGGCCCGAGGCATGGAGTATCTGGCCTCCAAGAAGTGCATACACCGA
GACCTGGCAGCCAGGAATGTCCTGGTGACAGAGGACAATGTGATGAAGAT
AGCAGACTTTGGCCTCGCACGGGACATTCACCACATCGACTACTATAAAA
AGACAACCAACGGCCGACTGCCTGTGAAGTGGATGGCACCCGAGGCATTA
TTTGACCGGATCTACACCCACCAGAGTGATGTGTGGTCTTTCGGGGTGCT
CCTGTGGGAGATCTTCACTCTGGGCGGCTCCCCATACCCCGGTGTGCCTG
TGGAGGAACTTTTCAAGCTGCTGAAGGAGGGTCACCGCATGGACAAGCCC
AGTAACTGCACCAACGAGCTGTACATGATGATGCGGGACTGCTGGCATGC
AGTGCCCTCACAGAGACCCACCTTCAAGCAGCTGGTGGAAGACCTGGACC
GCATCGTGGCCTTGACCTCCAACCAGGAGTACCTGGACCTGTCCATGCCC
CTGGACCAGTACTCCCCCAGCTTTCCCGACACCCGGAGCTCTACGTGCTC
CTCAGGGGAGGATTCCGTCTTCTCTCATGAGCCGCTGCCCGAGGAGCCCT
GCCTGCCCCGACACCCAGCCCAGCTTGCCAATGGCGGACTCAAACGCCGC
TGA

The primers used for cloning of SEQ ID Nos: 20 and 21 are shown as SEQ ID Nos: 22 to 23 below.

SEQ ID No: 22
CCGACAAAGAGATGGAGGTG (218_attB1_FGFR1_Virtuell)
SEQ ID No: 23
CAAAGAGATGGAGGTGCTTCAC (219_attB1_FGFR1_Virtuell)

SEQ ID No: 25 is the FGFR1 transcript of a further exemplary sample of a responder to FGFR1-inhibitor therapy.

SEQ ID No: 25
ATGGTGGGGTCGGTCATCGTCTACAAGATGAAGAGTGGTACCAAGAAGAG
TGACTTCCACAGCCAGATGGCTGTGCACAAGCTGGCCAAGAGCATCCCTC
TGCGCAGACAGGTGTCTGCTGACTCCAGTGCATCCATGAACTCTGGGGTT
CTTCTGGTTCGGCCATCACGGCTCTCCTCCAGTGGGACTCCCATGCTAGC
AGGGGTCTCTGAGTATGAGCTTCCCGAAGACCCTCGCTGGGAGCTGCCTC
GGGACAGACTGGTCTTAGGCAAACCCCTGGGAGAGGGCTGCTTTGGGCAG
GTGGTGTTGGCAGAGGCTATCGGGCTGGACAAGGACAAACCCAACCGTGT
GACCAAAGTGGCTGTGAAGATGTTGAAGTCGGACGCAACAGAGAAAGACT
TGTCAGACCTGATCTCAGAAATGGAGATGATGAAGATGATCGGGAAGCAT
AAGAATATCATCAACCTGCTGGGGGCCTGCACGCAGGATGGTCCCTTGTA
TGTCATCGTGGAGTATGCCTCCAAGGGCAACCTGCGGGAGTACCTGCAGG
CCCGGAGGCCCCCAGGGCTGGAATACTGCTACAACCCCAGCCACAACCCA
GAGGAGCAGCTCTCCTCCAAGGACCTGGTGTCCTGCGCCTACCAGGTGGC
CCGAGGCATGGAGTATCTGGCCTCCAAGAAGTGCATACACCGAGACCTGG
CAGCCAGGAATGTCCTGGTGACAGAGGACAATGTGATGAAGATAGCAGAC
TTTGGCCTCGCACGGGACATTCACCACATCGACTACTATAAAAAGACAAC
CAACGGCCGACTGCCTGTGAAGTGGATGGCACCCGAGGCATTATTTGACC
GGATCTACACCCACCAGAGTGATGTGTGGTCTTTCGGGGTGCTCCTGTGG
GAGATCTTCACTCTGGGCGGCTCCCCATACCCCGGTGTGCCTGTGGAGGA
ACTTTTCAAGCTGCTGAAGGAGGGTCACCGCATGGACAAGCCCAGTAACT
GCACCAACGAGCTGTACATGATGATGCGGGACTGCTGGCATGCAGTGCCC
TCACAGAGACCCACCTTCAAGCAGCTGGTGGAAGACCTGGACCGCATCGT
GGCCTTGACCTCCAACCAGGAGTACCTGGACCTGTCCATGCCCCTGGACC
AGTACTCCCCCAGCTTTCCCGACACCCGGAGCTCTACGTGCTCCTCAGGG
GAGGATTCCGTCTTCTCTCATGAGCCGCTGCCCGAGGAGCCCTGCCTGCC
CCGACACCCAGCCCAGCTTGCCAATGGCGGACTCAAACGCCGCTGA

Translation of SEQ ID No: 25 generates a protein comprising the amino acid sequence of SEQ ID No: 26.

SEQ ID No: 26
MVGSVIVYKMKSGTKKSDFHSQMAVHKLAKSIPLRRQVSADSSASMNSG
VLLVRPSRLSSSGTPMLAGVSEYELPEDPRWELPRDRLVLGKPLGEGCF
GQVVLAEAIGLDKDKPNRVTKVAVKMLKSDATEKDLSDLISEMEMMKMI
GKHKNIINLLGACTQDGPLYVIVEYASKGNLREYLQARRPPGLEYCYNP
SHNPEEQLSSKDLVSCAYQVARGMEYLASKKCIHRDLAARNVLVTEDNV
MKIADFGLARDIHHIDYYKKTTNGRLPVKWMAPEALFDRIYTHQSDVWS
FGVLLWEIFTLGGSPYPGVPVEELFKLLKEGHRMDKPSNCTNELYMMMR
DCWHAVPSQRPTFKQLVEDLDRIVALTSNQEYLDLSMPLDQYSPSFPDT
RSSTCSSGEDSVFSHEPLPEEPCLPRHPAQLANGGLKRR

The altered FGFR1 protein comprising the amino acid sequence of SEQ ID No: 26 lacks additional 21 amino acids due to the N-terminal truncation when compared to ΔEC-FGFR1 (SEQ ID No: 14). SEQ ID No: 26 is also referred to as ΔEC-21-FGFR1. The double-underlined residues in SEQ ID No: 26 are part of a shorter transmembrane domain, whereas the single-underlined residues form the kinase domain.

The forward primer used for cloning of SEQ ID No: 25 is shown as SEQ ID No: 27 below. SEQ ID No: 27-ATGGTGGGGTCGGTCAT (230_F_ΔEC-21-FGFR1). As reverse primer for cloning of SEQ ID No: 25, any reverse primer in exon 1 to 12 of FGFR1 can be used.

SEQ ID No: 28 is the FGFR1 transcript of a further exemplary sample of a responder to FGFR1-inhibitor therapy.

SEQ ID No: 28
ATGAAGAGTGGTACCAAGAAGAGTGACTTCCACAGCCAGATGGCTGTGC
ACAAGCTGGCCAAGAGCATCCCTCTGCGCAGACAGGTGTCTGCTGACTC
CAGTGCATCCATGAACTCTGGGGTTCTTCTGGTTCGGCCATCACGGCTC
TCCTCCAGTGGGACTCCCATGCTAGCAGGGGTCTCTGAGTATGAGCTTC
CCGAAGACCCTCGCTGGGAGCTGCCTCGGGACAGACTGGTCTTAGGCAA
ACCCCTGGGAGAGGGCTGCTTTGGGCAGGTGGTGTTGGCAGAGGCTATC
GGGCTGGACAAGGACAAACCCAACCGTGTGACCAAAGTGGCTGTGAAGA
TGTTGAAGTCGGACGCAACAGAGAAAGACTTGTCAGACCTGATCTCAGA
AATGGAGATGATGAAGATGATCGGGAAGCATAAGAATATCATCAACCTG
CTGGGGGCCTGCACGCAGGATGGTCCCTTGTATGTCATCGTGGAGTATG
CCTCCAAGGGCAACCTGCGGGAGTACCTGCAGGCCCGGAGGCCCCCAGG
GCTGGAATACTGCTACAACCCCAGCCACAACCCAGAGGAGCAGCTCTCC
TCCAAGGACCTGGTGTCCTGCGCCTACCAGGTGGCCCGAGGCATGGAGT
ATCTGGCCTCCAAGAAGTGCATACACCGAGACCTGGCAGCCAGGAATGT
CCTGGTGACAGAGGACAATGTGATGAAGATAGCAGACTTTGGCCTCGCA
CGGGACATTCACCACATCGACTACTATAAAAAGACAACCAACGGCCGAC
TGCCTGTGAAGTGGATGGCACCCGAGGCATTATTTGACCGGATCTACAC
CCACCAGAGTGATGTGTGGTCTTTCGGGGTGCTCCTGTGGGAGATCTTC
ACTCTGGGCGGCTCCCCATACCCCGGTGTGCCTGTGGAGGAACTTTTCA
AGCTGCTGAAGGAGGGTCACCGCATGGACAAGCCCAGTAACTGCACCAA
CGAGCTGTACATGATGATGCGGGACTGCTGGCATGCAGTGCCCTCACAG
AGACCCACCTTCAAGCAGCTGGTGGAAGACCTGGACCGCATCGTGGCCT
TGACCTCCAACCAGGAGTACCTGGACCTGTCCATGCCCCTGGACCAGTA
CTCCCCCAGCTTTCCCGACACCCGGAGCTCTACGTGCTCCTCAGGGGAG
GATTCCGTCTTCTCTCATGAGCCGCTGCCCGAGGAGCCCTGCCTGCCCC
GACACCCAGCCCAGCTTGCCAATGGCGGACTCAAACGCCGCTGA

Translation of SEQ ID No: 28 generates a protein comprising the amino acid sequence of SEQ ID No: 29.

SEQ ID No: 29
MKSGTKKSDFHSQMAVHKLAKSIPLRRQVSADSSASMNSGVLLVRPSRL
SSSGTPMLAGVSEYELPEDPRWELPRDRLVLGKPLGEGCFGQVVLAEAI
GLDKDKPNRVTKVAVKMLKSDATEKDLSDLISEMEMMKMIGKHKNIINL
LGACTQDGPLYVIVEYASKGNLREYLQARRPPGLEYCYNPSHNPEEQLS
SKDLVSCAYQVARGMEYLASKKCIHRDLAARNVLVTEDNVMKIADFGLA
RDIHHIDYYKKTTNGRLPVKWMAPEALFDRIYTHQSDVWSFGVLLWEIF
TLGGSPYPGVPVEELFKLLKEGHRMDKPSNCTNELYMMMRDCWHAVPSQ
RPTFKQLVEDLDRIVALTSNQEYLDLSMPLDQYSPSFPDTRSSTCSSGE
DSVFSHEPLPEEPCLPRHPAQLANGGLKRR

The altered FGFR1 protein comprising the amino acid sequence of SEQ ID No: 29 lacks 30 amino acids when compared to ΔEC-FGFR1 (SEQ ID No: 14). SEQ ID No: 29 is also referred to as ΔEC-30-FGFR1. Due to the N-terminal truncation, the protein comprising the amino acid sequence of SEQ ID No: 29 has no IG-like domain, but a kinase domain. The underlined residues in SEQ ID No: 29 above form the kinase domain.

The forward primer used for cloning of SEQ ID No: 28 is shown as SEQ ID No: 30 below. SEQ ID No: 30-ATGAAGAGTGGTACCAAGAAGAGTG (231_F_ΔEC-30-FGFR1). As reverse primer for cloning of SEQ ID No: 28, any reverse primer in exon 1 to 12 of FGFR1 can be used.

SEQ ID No: 31 is the FGFR1 transcript of a further exemplary sample of a responder to FGFR1-inhibitor therapy.

SEQ ID No: 31
ATGCTAGCAGGGGTCTCTGAGTATGAGCTTCCCGAAGACCCTCGCTGGG
AGCTGCCTCGGGACAGACTGGTCTTAGGCAAACCCCTGGGAGAGGGCTG
CTTTGGGCAGGTGGTGTTGGCAGAGGCTATCGGGCTGGACAAGGACAAA
CCCAACCGTGTGACCAAAGTGGCTGTGAAGATGTTGAAGTCGGACGCAA
CAGAGAAAGACTTGTCAGACCTGATCTCAGAAATGGAGATGATGAAGAT
GATCGGGAAGCATAAGAATATCATCAACCTGCTGGGGGCCTGCACGCAG
GATGGTCCCTTGTATGTCATCGTGGAGTATGCCTCCAAGGGCAACCTGC
GGGAGTACCTGCAGGCCCGGAGGCCCCCAGGGCTGGAATACTGCTACAA
CCCCAGCCACAACCCAGAGGAGCAGCTCTCCTCCAAGGACCTGGTGTCC
TGCGCCTACCAGGTGGCCCGAGGCATGGAGTATCTGGCCTCCAAGAAGT
GCATACACCGAGACCTGGCAGCCAGGAATGTCCTGGTGACAGAGGACAA
TGTGATGAAGATAGCAGACTTTGGCCTCGCACGGGACATTCACCACATC
GACTACTATAAAAAGACAACCAACGGCCGACTGCCTGTGAAGTGGATGG
CACCCGAGGCATTATTTGACCGGATCTACACCCACCAGAGTGATGTGTG
GTCTTTCGGGGTGCTCCTGTGGGAGATCTTCACTCTGGGCGGCTCCCCA
TACCCCGGTGTGCCTGTGGAGGAACTTTTCAAGCTGCTGAAGGAGGGTC
ACCGCATGGACAAGCCCAGTAACTGCACCAACGAGCTGTACATGATGAT
GCGGGACTGCTGGCATGCAGTGCCCTCACAGAGACCCACCTTCAAGCAG
CTGGTGGAAGACCTGGACCGCATCGTGGCCTTGACCTCCAACCAGGAGT
ACCTGGACCTGTCCATGCCCCTGGACCAGTACTCCCCCAGCTTTCCCGA
CACCCGGAGCTCTACGTGCTCCTCAGGGGAGGATTCCGTCTTCTCTCAT
GAGCCGCTGCCCGAGGAGCCCTGCCTGCCCCGACACCCAGCCCAGCTTG
CCAATGGCGGACTCAAACGCCGCTGA

Translation of SEQ ID No: 31 generates a protein comprising the amino acid sequence of SEQ ID No: 32.

SEQ ID No: 32
MLAGVSEYELPEDPRWELPRDRLVLGKPLGEGCFGQVVLAEAIGLDKDK
PNRVTKVAVKMLKSDATEKDLSDLISEMEMMKMIGKHKNIINLLGACTQ
DGPLYVIVEYASKGNLREYLQARRPPGLEYCYNPSHNPEEQLSSKDLVS
CAYQVARGMEYLASKKCIHRDLAARNVLVTEDNVMKIADFGLARDIHHI
DYYKKTTNGRLPVKWMAPEALFDRIYTHQSDVWSFGVLLWEIFTLGGSP
YPGVPVEELFKLLKEGHRMDKPSNCTNELYMMMRDCWHAVPSQRPTFKQ
LVEDLDRIVALTSNQEYLDLSMPLDQYSPSFPDTRSSTCSSGEDSVFSH
EPLPEEPCLPRHPAQLANGGLKRR

The altered FGFR1 protein comprising the amino acid sequence of SEQ ID No: 32 lacks 85 amino acids when compared to ΔEC-FGFR1 (SEQ ID No: 14). SEQ ID No: 32 is also referred to as ΔEC-85-FGFR1. The protein comprising the amino acid sequence of SEQ ID No: 32 has no IG-like domain due to the N-terminal truncation, but a kinase domain. The underlined residues in SEQ ID No: 32 form the kinase domain.

The forward primer used for cloning of SEQ ID No: 31 is shown as SEQ ID No: 33 below. SEQ ID No: 33-CATGCTAGCAGGGGTCTCT (232_F_ΔEC-85-FGFR1). As reverse primer for cloning of SEQ ID No: 25, any reverse primer in exon 1 to 12 of FGFR1 can be used.

SEQ ID No: 34 is the FGFR1 transcript of a further exemplary sample of a responder to FGFR1-inhibitor therapy.

SEQ ID No: 34
ATGCTAGCAGGGGTCTCTGAGTATGAGCTTCCCGAAGACCCTCGCTGGG
AGCTGCCTCGGGACAGACTGGTCTTAGGCAAACCCCTGGGAGAGGGCTG
CTTTGGGCAGGTGGTGTTGGCAGAGGCTATCGGGCTGGACAAGGACAAA
CCCAACCGTGTGACCAAAGTGGCTGTGAAGATGTTGAAGTCGGACGCAA
CAGAGAAAGACTTGTCAGACCTGATCTCAGAAATGGAGATGATGAAGAT
GATCGGGAAGCATAAGAATATCATCAACCTGCTGGGGGCCTGCACGCAG
GATGGTCCCTTGTATGTCATCGTGGAGTATGCCTCCAAGGGCAACCTGC
GGGAGTACCTGCAGGCCCGGAGGCCCCCAGGGCTGGAATACTGCTACAA
CCCCAGCCACAACCCAGAGGAGCAGCTCTCCTCCAAGGACCTGGTGTCC
TGCGCCTACCAGGTGGCCCGAGGCATGGAGTATCTGGCCTCCAAGAAGT
GCATACACCGAGACCTGGCAGCCAGGAATGTCCTGGTGACAGAGGACAA
TGTGATGAAGATAGCAGACTTTGGCCTCGCACGGGACATTCACCACATC
GACTACTATAAAAAGACAACCAACGGCCGACTGCCTGTGAAGTGGATGG
CACCCGAGGCATTATTTGACCGGATCTACACCCACCAGAGTGATGTGTG
GTCTTTCGGGGTGCTCCTGTGGGAGATCTTCACTCTGGGCGGCTCCCCA
TACCCCGGTGTGCCTGTGGAGGAACTTTTCAAGCTGCTGAAGGAGGGTC
ACCGCATGGACAAGCCCAGTAACTGCACCAACGAGCTGTACATGATGAT
GCGGGACTGCTGGCATGCAGTGCCCTCACAGAGACCCACCTTCAAGCAG
CTGGTGGAAGACCTGGACCGCATCGTGGCCTTGACCTCCAACCAGGAGT
ACCTGGACCTGTCCATGCCCCTGGACCAGTACTCCCCCAGCTTTCCCGA
CACCCGGAGCTCTACGTGCTCCTCAGGGGAGGATTCCGTCTTCTCTCAT
GAGCCGCTGCCCGAGGAGCCCTGCCTGCCCCGACACCCAGCCCAGCTTG
CCAATGGCGGACTCAAACGCCGCTGA

Translation of SEQ ID No: 34 generates a protein comprising the amino acid sequence of SEQ ID No: 35.

SEQ ID No: 35
MLKSDATEKDLSDLISEMEMMKMIGKHKNIINLLGACTQDGPLYVIVEY
ASKGNLREYLQARRPPGLEYCYNPSHNPEEQLSSKDLVSCAYQVARGME
YLASKKCIHRDLAARNVLVTEDNVMKIADFGLARDIHHIDYYKKTTNGR
LPVKWMAPEALFDRIYTHQSDVWSFGVLLWEIFTLGGSPYPGVPVEELF
KLLKEGHRMDKPSNCTNELYMMMRDCWHAVPSQRPTFKQLVEDLDRIVA
LTSNQEYLDLSMPLDQYSPSFPDTRSSTCSSGEDSVFSHEPLPEEPCLP
RHPAQLANGGLKRR

The altered FGFR1 protein comprising the amino acid sequence of SEQ ID No: 35 lacks 144 amino acids when compared to ΔEC-FGFR1 (SEQ ID No: 14). Therefore, SEQ ID No: 35 is also referred to as ΔEC-144-FGFR1. Due to the N-terminal truncation, the protein comprising the amino acid sequence of SEQ ID No: 35 has no IG-like domain, and most likely no functional kinase domain. However, residues that form a part of the kinase domain of the amino acid sequence of SEQ ID No: 35 are underlined.

The forward primer used for cloning of SEQ ID No: 34 is shown as SEQ ID No: 36 below. SEQ ID No: 36-ATGCTAGCAGGGGTCTCTGA (233_F_ΔEC-144-FGFR1). As reverse primer for cloning of SEQ ID No: 25, any reverse primer in exon 1 to 12 of FGFR1 can be used.

SEQ ID No: 37 shows wild type FGFR1 (NM_015850)

SEQ ID No: 37
ATGTGGAGCTGGAAGTGCCTCCTCTTCTGGGCTGTGCTGGTCACAGCCA
CACTCTGCACCGCTAGGCCGTCCCCGACCTTGCCTGAACAAGCCCAGCC
CTGGGGAGCCCCTGTGGAAGTGGAGTCCTTCCTGGTCCACCCCGGTGAC
CTGCTGCAGCTTCGCTGTCGGCTGCGGGACGATGTGCAGAGCATCAACT
GGCTGCGGGACGGGGTGCAGCTGGCGGAAAGCAACCGCACCCGCATCAC
AGGGGAGGAGGTGGAGGTGCAGGACTCCGTGCCCGCAGACTCCGGCCTC
TATGCTTGCGTAACCAGCAGCCCCTCGGGCAGTGACACCACCTACTTCT
CCGTCAATGTTTCAGATGCTCTCCCCTCCTCGGAGGATGATGATGATGA
TGATGACTCCTCTTCAGAGGAGAAAGAAACAGATAACACCAAACCAAAC
CCCGTAGCTCCATATTGGACATCCCCAGAAAAGATGGAAAAGAAATTGC
ATGCAGTGCCGGCTGCCAAGACAGTGAAGTTCAAATGCCCTTCCAGTGG
GACCCCAAACCCCACACTGCGCTGGTTGAAAAATGGCAAAGAATTCAAA
CCTGACCACAGAATTGGAGGCTACAAGGTCCGTTATGCCACCTGGAGCA
TCATAATGGACTCTGTGGTGCCCTCTGACAAGGGCAACTACACCTGCAT
TGTGGAGAATGAGTACGGCAGCATCAACCACACATACCAGCTGGATGTC
GTGGAGCGGTCCCCTCACCGGCCCATCCTGCAAGCAGGGTTGCCCGCCA
ACAAAACAGTGGCCCTGGGTAGCAACGTGGAGTTCATGTGTAAGGTGTA
CAGTGACCCGCAGCCGCACATCCAGTGGCTAAAGCACATCGAGGTGAAT
GGGAGCAAGATTGGCCCAGACAACCTGCCTTATGTCCAGATCTTGAAGA
CTGCTGGAGTTAATACCACCGACAAAGAGATGGAGGTGCTTCACTTAAG
AAATGTCTCCTTTGAGGACGCAGGGGAGTATACGTGCTTGGCGGGTAAC
TCTATCGGACTCTCCCATCACTCTGCATGGTTGACCGTTCTGGAAGCCC
TGGAAGAGAGGCCGGCAGTGATGACCTCGCCCCTGTACCTGGAGATCAT
CATCTATTGCACAGGGGCCTTCCTCATCTCCTGCATGGTGGGGTCGGTC
ATCGTCTACAAGATGAAGAGTGGTACCAAGAAGAGTGACTTCCACAGCC
AGATGGCTGTGCACAAGCTGGCCAAGAGCATCCCTCTGCGCAGACAGGT
AACAGTGTCTGCTGACTCCAGTGCATCCATGAACTCTGGGGTTCTTCTG
GTTCGGCCATCACGGCTCTCCTCCAGTGGGACTCCCATGCTAGCAGGGG
TCTCTGAGTATGAGCTTCCCGAAGACCCTCGCTGGGAGCTGCCTCGGGA
CAGACTGGTCTTAGGCAAACCCCTGGGAGAGGGCTGCTTTGGGCAGGTG
GTGTTGGCAGAGGCTATCGGGCTGGACAAGGACAAACCCAACCGTGTGA
CCAAAGTGGCTGTGAAGATGTTGAAGTCGGACGCAACAGAGAAAGACTT
GTCAGACCTGATCTCAGAAATGGAGATGATGAAGATGATCGGGAAGCAT
AAGAATATCATCAACCTGCTGGGGGCCTGCACGCAGGATGGTCCCTTGT
ATGTCATCGTGGAGTATGCCTCCAAGGGCAACCTGCGGGAGTACCTGCA
GGCCCGGAGGCCCCCAGGGCTGGAATACTGCTACAACCCCAGCCACAAC
CCAGAGGAGCAGCTCTCCTCCAAGGACCTGGTGTCCTGCGCCTACCAGG
TGGCCCGAGGCATGGAGTATCTGGCCTCCAAGAAGTGCATACACCGAGA
CCTGGCAGCCAGGAATGTCCTGGTGACAGAGGACAATGTGATGAAGATA
GCAGACTTTGGCCTCGCACGGGACATTCACCACATCGACTACTATAAAA
AGACAACCAACGGCCGACTGCCTGTGAAGTGGATGGCACCCGAGGCATT
ATTTGACCGGATCTACACCCACCAGAGTGATGTGTGGTCTTTCGGGGTG
CTCCTGTGGGAGATCTTCACTCTGGGCGGCTCCCCATACCCCGGTGTGC
CTGTGGAGGAACTTTTCAAGCTGCTGAAGGAGGGTCACCGCATGGACAA
GCCCAGTAACTGCACCAACGAGCTGTACATGATGATGCGGGACTGCTGG
CATGCAGTGCCCTCACAGAGACCCACCTTCAAGCAGCTGGTGGAAGACC
TGGACCGCATCGTGGCCTTGACCTCCAACCAGGAGTACCTGGACCTGTC
CATGCCCCTGGACCAGTACTCCCCCAGCTTTCCCGACACCCGGAGCTCT
ACGTGCTCCTCAGGGGAGGATTCCGTCTTCTCTCATGAGCCGCTGCCCG
AGGAGCCCTGCCTGCCCCGACACCCAGCCCAGCTTGCCAATGGCGGACT
CAAACGCCGCTGA

### Examples

Certain aspects and embodiments of the invention will now be illustrated by way of example and with reference to the description, figures and tables set out herein. Such examples of the methods, uses and other aspects of the present invention are representative only, and should not be taken to limit the scope of the present invention to only such representative examples.

The examples show:

### Example 1: Identification of Head-to-Head Followed by Tail-to-Tail Intra-Chromosomal Breaks within the 8p Arm by a Breakage-Fusion-Bridge-Like Mechanism in Squamous Cell Lung Cancer

The inventors analyzed whether a distinct 8p11 amplified genome structure, such as heterogeneous 8p11-8p12 amplification patterns, leads to genotypic and phenotypic FGFR1-dependency, i.e. the responsiveness of FGFR1-amplified tumors to FGFR inhibitor (FGFRi) therapy. Specifically, the inventors first analyzed 8p11-12-amplified squamous cell lung carcinomas with unknown responsiveness to FGFR inhibition.

Human lung tumor specimens (initial study cohort): The inventors collected in total 27 fresh-frozen squamous cell lung cancer tumor (SQLC) samples, which were provided by multiple collaborating institutions as fresh-frozen tissue specimen, frozen sections or as genomic DNA extracted from fresh-frozen material. All tumor samples were pathologically assessed to have a purity of at least 60% and no extensive signs of necrosis. Additionally, these tumor samples were reviewed by at least two independent expert pathologists and the diagnosis of SQLC was histomorphologically confirmed by H&E staining and immunohistochemistry. For immunohistochemistry, tissues were fixed in 4% PBS-buffered formalin and embedded in paraffin (FFPE). Immunohistochemistry was performed as described previously on 3 μm slides with specific antibodies for pFGFR1 (Abnova, Y154).

Matching normal material was provided in the form of EDTA-anticoagulated blood or adjacent non-tumorigenic lung tissue. The matched normal tissue was confirmed to be free of tumor contaminants by pathological assessment. Furthermore, tumor and matching normal material were confirmed to be acquired from the same patient by SNP 6.0 array and sequencing analyses. Human tumour samples were obtained from patients under IRB-approved protocols following written informed consent and Patient material was stored at −80° C.

Genome and transcriptome sequencing: The data is processed by aligning sequencing reads to a reference genome (NCBI build 37/hg19) using bwa-mem (0.7.13-r1126; https://github.com/lh3/bwa), masking potential PCR-duplicates and regions of overlapping read pairs, and the collection of various count statistics which are used to call mutations and, for whole genomes, genomic rearrangements. Finally, Sclust is used for sample purity estimation and copy number analysis. RNA-seq data was analysed using TRUP.

ARCHER sequencing: RNA was extracted from FFPE material using the Maxwell RSC in combination with the Maxwell RSC RNA FFPE Kit (Promega) according to the manufacturer's instruction. Removal of genomic DNA was performed with the TURBO DNA-free Kit (Thermo Fisher Scientific) and both tRNA and RNA were quantified using the Qubit RNA HS Assay Kit (Thermo Fisher). Analysis of RNA integrity was done with the 4200 TapeStation System (Agilent Technologies, Santa Clara, CA, USA).

For fusion detection, the Archer FusionPlex Lung Panel (ArcherDX) was used according to manufacturer's instructions with 200 ng tRNA input for library preparation. Using the KAPA Library Quantification Kit (Roche, Pleasanton, CA, USA), purified libraries were quantified. Sequencing was performed on the Nextseq System (Illumina, San Diego, CA, USA) and results were analyzed with the Archer Suite Analysis v 6.2.7 (ArcherDX). Since the Archer Suite Analysis software is not able to label inversions, bam files were extracted and loaded directly in the Integrative Genomics Viewer (IGV) to visualize tail-to-tail breaks.

Computational Analysis: Copy numbers were visualized with GISTIC (Ref. GISTIC2.0 facilitates sensitive and confident localization of the targets of focal somatic copy-number alteration in human cancers) and ROBOCOP. To summaries the copy number information in the FGFR1 region across samples, a view window of 2 MB around the FGFR1 locus was split into 10′000 bins. The available chromosome segment-based copy number data were then mapped onto these bins for each of the samples. Next, the per-bin means of the copy numbers for all samples within a certain condition were calculated and the rolling mean with a window size of 10 (for whole genome sequencing data) and 500 of these numbers was determined to reduce noise in the CAGE data. The process of determining ROlling Binmeans Of COPy numbers is defined here as the ROBOCOP algorithm. Given a chromosome segmentation and sequence of segment copy number values n at the (amplified) FGFR1 locus, the approach estimates an approximate copy number sequence “n′” by applying palindromic BFB transformations to an initial wild-type segmentation. A similarity/distance between the sequences is measured as the Poisson likelihood of observing sequence n given the approximation n′. Using the provided.Java application, BFB inference was done in two steps: (1) Estimating n′ (java bfb.BFB_Algo counts: n mode: substring model: Poisson maxError: o) and (2) converting n′ into a sequence of BFB transformations (java bfb.BFB_Algo counts: n′ mode: search). Interestingly, only amplicons with a BFB approximation of at least eight segments were considered as high confidence (n=11), while amplicons with less than eight segments and/or no support by fold-back inversions were too ambiguous to reliably infer BFB as the underlying mechanism (n=14).

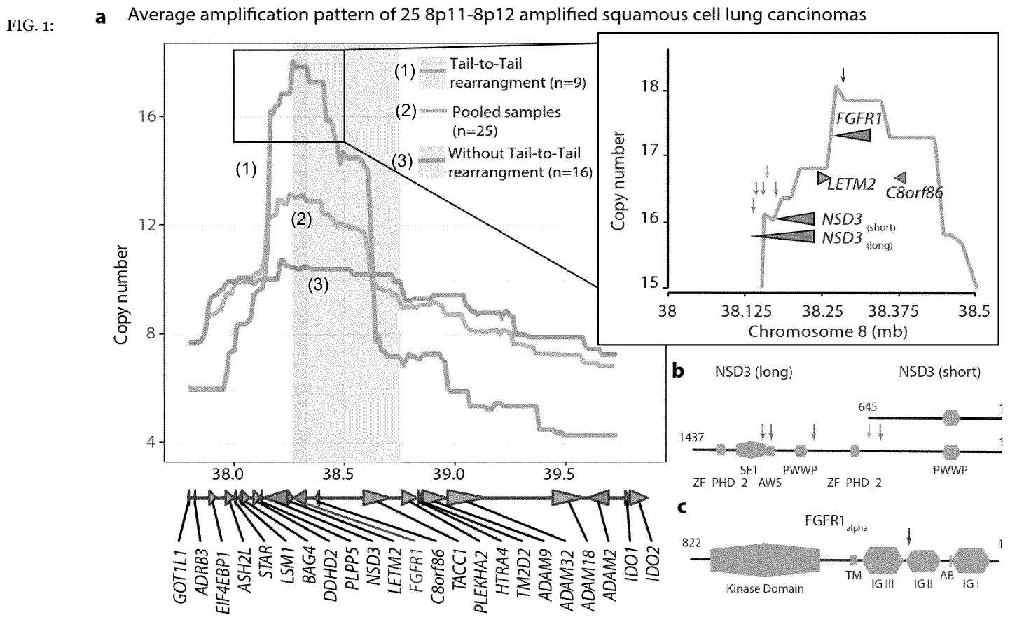

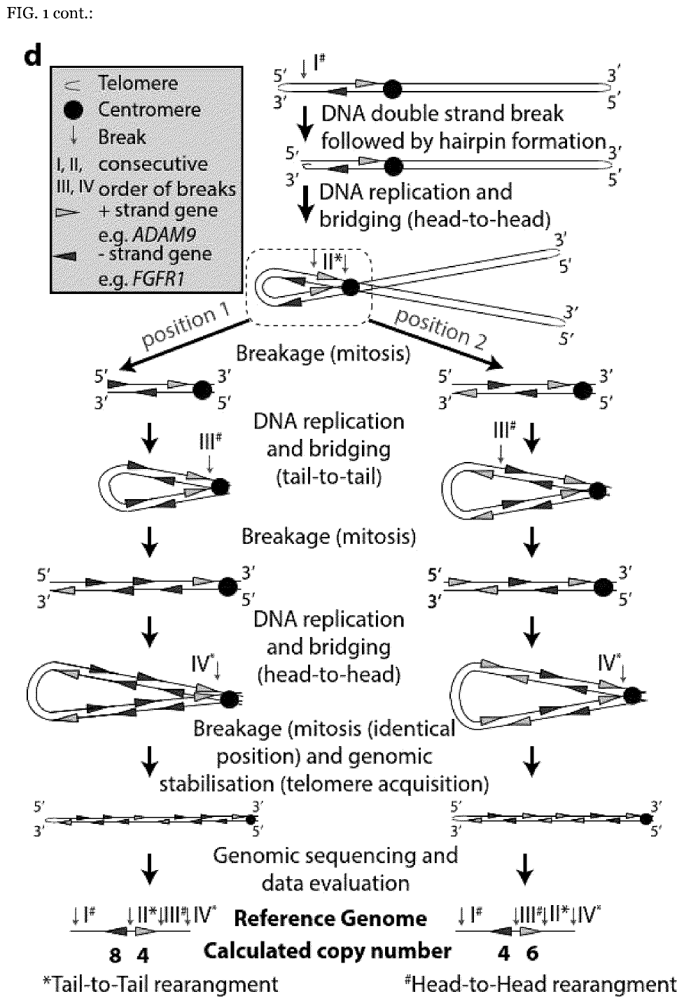

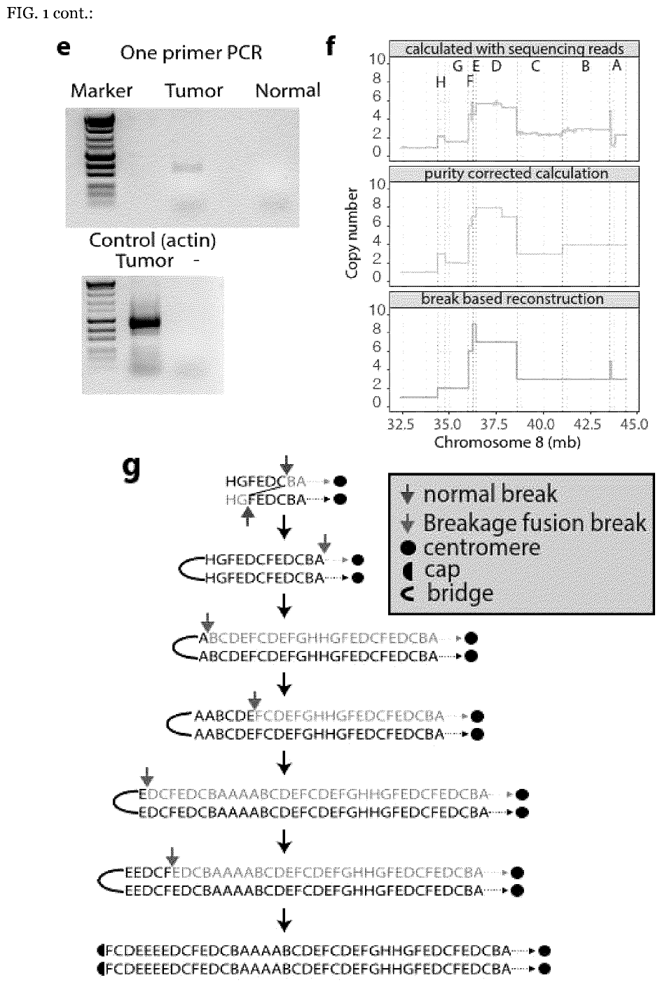

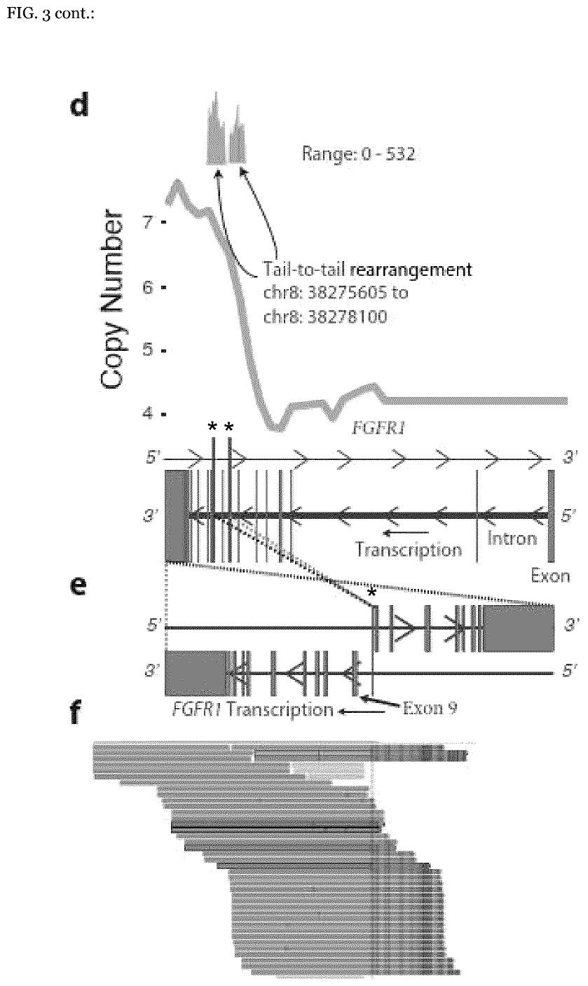

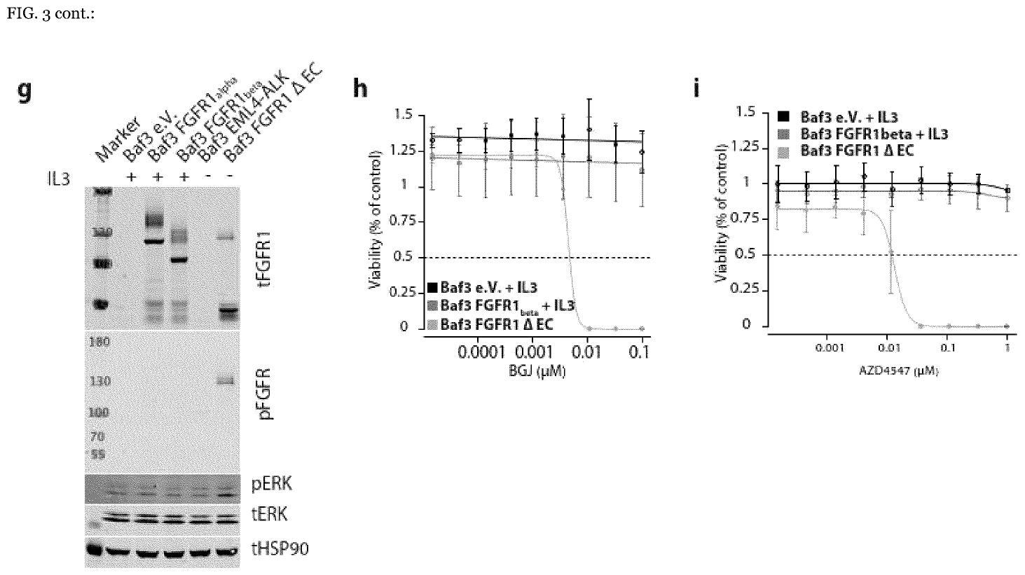

The inventors performed whole-genome sequencing and transcriptome (22 of 25) sequencing of tumors and their matched normal tissue. As expected, plotting the average copy number of all 25 samples revealed that the amplification was centered on FGFR1 and the adjacent NSD3 gene (FDR q=1.3×10⁻³⁸) (FIG. 1a). In order to decode the precise genomic architecture of the 8p11-12 amplicon, the inventors inspected all occurring rearrangements and termed them according to their orientation, as compared to the reference genome (normal, head-to-head or tail-to-tail). The inventors found that only the amplicons with tail-to-tail breaks close to the transcriptional start side of FGFR1 drove the amplification pattern that was centered around FGFR1/NSD3. The inventors identified 9 samples, in which tail-to-tail breaks occurred within a 400-kilobase (kb) region upstream of FGFR1 (36%). These samples exhibited a focal amplicon of 550 kb that was focused on only one gene, FGFR1, whereas the amplified region was three times larger (1.7 mb) in tumors without distinct tail-to-tail breaks (FIG. 1a). In 5 out of the 9 specimens (56%), in which the amplicon centered on FGFR1, a second break that led to the focal amplification, was within the gene NSD3 (or WHSC1L1). These breaks were mainly head-to-head breaks (4 out of 5) and led to deletion of exons 13 to 23 of NSD3 (FIG. 1b). Tumors with head-to-head breaks within NSD3 exhibited increased expression of the short isoform of NSD3, which lacks the catalytic SET domain and has been reported to be non-oncogenic. On the contrary, a tail-to-tail break within FGFR1 occurred in another tumor amplifying particularly the intracellular part of FGFR1 (FIG. 1c). The implications of this break will be described below (FIG. 3). Thus, in 9 of 25 8p-amplified squamous cell lung cancer specimens, tail-to-tail rearrangements caused focal amplifications that were centered exclusively on FGFR1. In 4 of these tumors, the inventors found predominant expression of a non-oncogenic version of NSD3 lacking the SET domain-encoding exons resulting from deleterious genomic events (FIG. 1b).

Further detailed analyses indicated that the pattern of genomic rearrangements on 8p originated from Breakage-Fusion-Bridge (BFB) cycles with characteristic features of telomeric loss, clipped read orientation, copy number variation and at least eight copy number segments (FIG. 1d). In all 25 FGFR1-amplified cases a telomeric loss was observed that was frequently accompanied by intra-chromosomal head-to-head and tail-to-tail fusions (detected as head-to-head or tail-to-tail breaks by clipped read orientation) (FIG. 1d, e). The inventors were thus able to reliably identify BFBs as the underlying cause of 8p amplification in 44% (11 of 25) of the tumors. In 56% of the samples a BFB mechanism was uncertain (48%) or excluded (8%), because of low segmentation or no support from the observed rearrangements. Finally, again confirming a BFB mechanism, the inventors were able to correctly reconstruct the precise 8p amplicon in one tumor considering only the observed genomic breaks and following the assembly logic of BFB as the underlying mechanism (FIG. 1f, g). In summary, 8p amplifications in squamous cell lung cancer are caused by breakage fusion bridges in the majority of cases. Intra-chromosomal tail-to-tail breaks close to the FGFR1 transcription start side followed by head-to-head breaks in or close to NSD3 cause focal and FGFR1-centered amplification.

In summary, the inventors showed that 8p11-8p12 amplifications are driven by a BFB-like mechanism. Intra-chromosomal tail-to-tail breaks close to the FGFR1 transcription start side cause focal amplification and are associated with FGFR inhibitor sensitivity.

### Example 2: Confirmation of a Breakage-Fusion-Bridge Mechanism Forming 8p11-p12-Amplified Lung Cancer Tumors and the Underlying Mechanism of FGFR1-Inhibitor Dependency

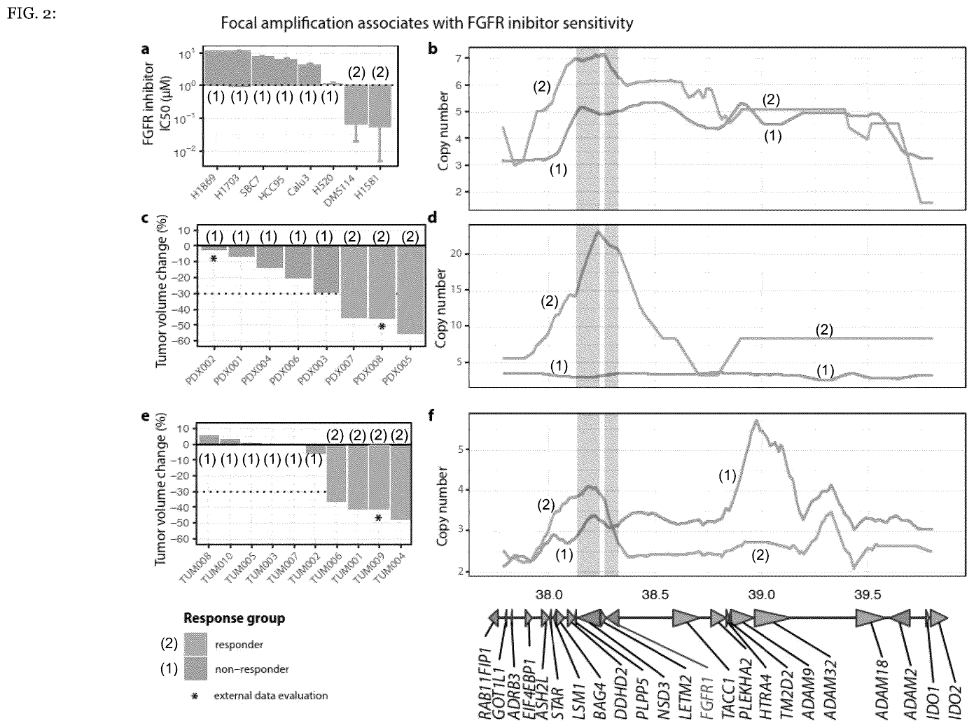

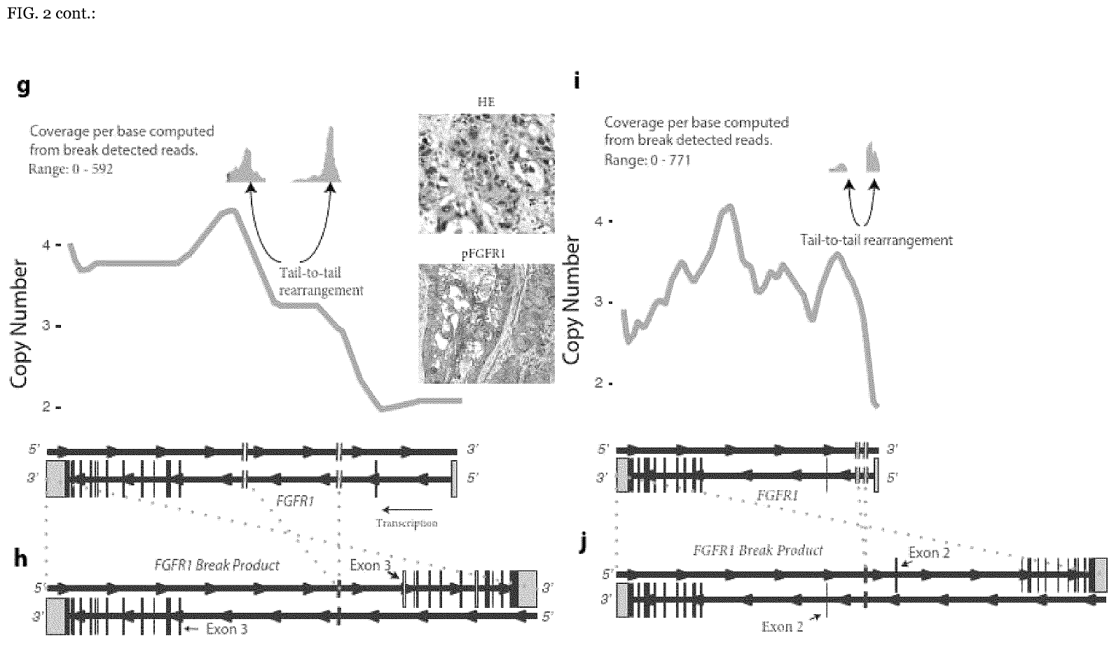

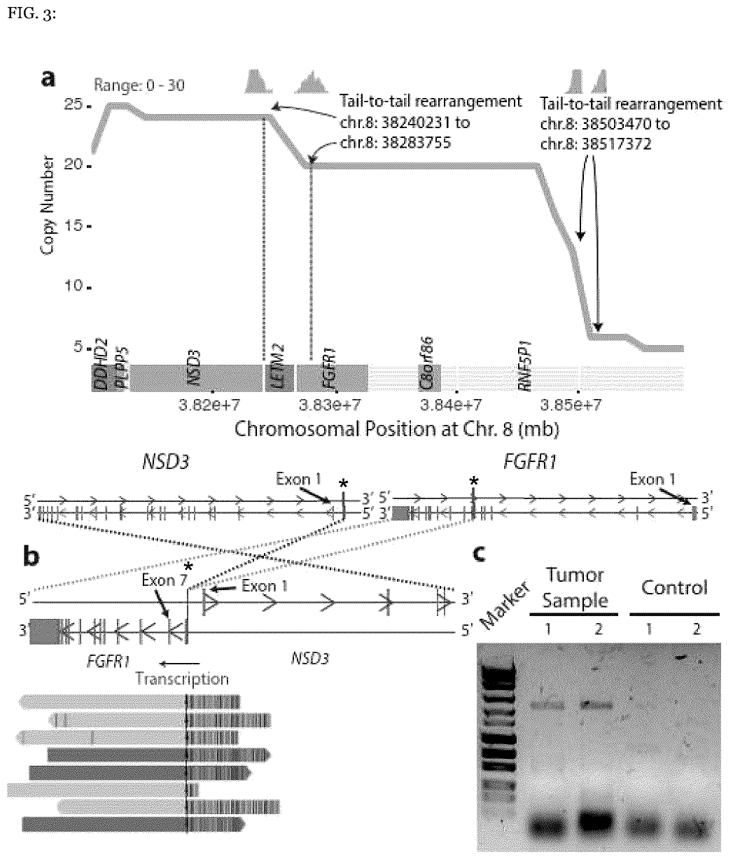

To gain a deeper understanding of 8p11-8p12-amplified lung cancer tumors and to link specific patterns of the 8p12-8p11 amplicon structure with FGFR1 dependency, the inventors analyzed an independent cohort of 26 8p-amplified tumor specimens with known sensitivity to FGFR inhibition. In detail, the inventors analyzed 8 FGFR1-amplified lung cancer cell lines (FIG. 2a, b), of which 6 were resistant to FGFR inhibition with the small-molecule inhibitors, BG.J398 and AZD4547 (half-growth-inhibitory concentration, or GI50, >1 μM) and two cell lines were sensitive (GI50<1 μM, H1581 and DMS114) (FIG. 2a). Confirming the inventors' hypothesis that the specific architecture of the 8p amplicon determines FGFR dependency, the amplicon centered on FGFR1 and NSD3 in the sensitive cell lines, while in the resistant cell lines the amplification was neither focal nor centered on FGFR1 (FIG. 2b). Of note, the inventors found one destructive NSD3 breakpoint in the most sensitive cell line H1581, rearranging exon 15 of NSD3 to an out of frame fusion with exon 10 of ANK1.

Furthermore, the inventors analysed patient-derived 8p12-8p11-amplified squamous cell lung cancer xenotransplant models. In total, the inventors performed FGFR1 fluorescence in situ hybridization (FISH) on 35 patient derived xenograft (PDX) models from EpoBerlin, 39 PDX models from Crown Bioscience Inc., 3 samples of Moro Massimo as previously described. In addition, the inventors established 8 PDX models by implanting subcutaneously 2-3 mm³ tumor pieces from biopsies (transported in DMEM media) together with 5-10 μl of Matrigel (Corning) in NSG mice. The inventors identified eight FGFR1 amplified models and performed CAGE sequencing. CAGE sequenced samples confirming FGFR1 amplification were daily oral treated with BG.J398 (Tocris Bioscience, 20 mg/kg, dissolved in 33% PEG300, 5% Glucose, prepare stock freshly every week and store at 4° C.) or vehicle (33% PEG300, 5% Glucose) with at least three animals per group. Three of the 8 patient derived xenotransplant (PDX) models were sensitive to FGFR inhibition (FIG. 2c). Analysis of chromosomal gene copy number extracted from genomic sequencing data again revealed a pattern of amplification that was centered on NSD3 and FGFR1 in the sensitive PDX models (FIG. 2d). Of note, the inventors observed in one sensitive model a deleterious head-to-head NSD3 rearrangement, deleting exons 9 to 23, but not in any of the insensitive models. By contrast, in insensitive PDX models 8p amplification was not centered on FGFR1 nor focal within the 8p11-8p12 locus and the inventors found a destructive head-to-head rearrangement affecting the FGFR1 gene, deleting exons 6 to 18 in one insensitive PDX, but not in any of the sensitive models.

Finally, the inventors analyzed biopsy specimens obtained from 10 patients with FGFR1-amplified squamous cell lung cancer. The samples were formalin fixed biopsies, taken before treatment. Eight of these patients had been treated with the FGFR-Inhibitor, BG.J398, as part of a phase I clinical trial (FIG. 2e) and one patient had responded to off-label use of pazopanib, a multi-kinase inhibitor that also inhibits FGFR. Of these 10 patients, 4 had experienced a partial response lasting for 9-17 months (defined as tumor shrinkage of at least 30% of the maximal tumor diameter) and 6 patients had progressive disease under treatment with an FGFR inhibitor. Given the low amount of available DNA from patient samples, the inventors developed a hybrid capture based sequencing assay tailored to cover much of the genomic 8p11-8p12 locus, as well as additional 226 genes. The inventors sequenced the 10 clinical specimens with an average sequencing depth of 470×using this approach. The inventors found three different PIK3CA mutations (G118S, E545K and H1047R) in addition to non-focal gain of 8p in 3 of 5 tumor specimens obtained from patients who had not experienced a response to the FGFR-inhibitor, BG.J398. Generally, patients who had not responded to FGFR inhibition, exhibited amplification centered on ADAM-family genes (FIG. 2e, f). By contrast, detailed analyses of chromosomal gene copy number revealed focal amplifications centered on FGFR1/NSD3 in patients with a measurable response to FGFR inhibition (FIG. 2e, f). Furthermore, the inventors identified one deleterious head-to-head break within NSD3 in the responding patient group (1 in 9 samples, deleting exons 6-23).

In a pooled analysis of patients treated with FGFR inhibitors, PDX models and cell lines, the inventors found that focal and FGFR1-centered amplification was induced by tail-to-tail breaks close to the FGFR1 transcriptional start side. These breaks occurred in 7 out of 9 (78%) “responders” (i.e., tumors/cell lines that were sensitive to FGFR inhibition) and 3 out of 12 (25%) non-responders (i.e., insensitive tumors/cell lines, for 5 cell line samples only whole exome sequencing data were available). Thus, tail-to-tail breaks close to the transcription start site of FGFR1 induce focal amplification and were associated with sensitivity to FGFR inhibition (p=0.03, Fisher's exact test). Furthermore, the inventors found three destructive NSD3 breaks in sensitive samples (33%), deleting exons 6 to 23, but not in any of the insensitive models.

In tumors obtained from two of the four sensitive patients, the inventors identified tail-to-tail rearrangements deleting the first exons of FGFR1, including the canonical ATG start codon (FIG. 2g-j), thus suggesting a possibly deleterious genomic event. In the first patient, the rearrangement occurred between exons 2 and 3 (FIG. 2h). The break in the second patient occurred between exons 1 and 2 (FIG. 2j). The inventors therefore speculated that the remaining part of the ORF of FGFR1 might be preserved by an alternative start codon downstream. In support of this notion, the inventors were able to obtain tissue of one of these two specimens, which stained positive for pFGFR1, thus indicating intact and activated FGFR1 signaling (FIG. 2g, right panel).

### Example 3: Identification of Intra-Chromosomal Tail-to-Tail Break from the Non-Coding Region Between FGFR1/NSD3 to FGFR1

As an additional support for the inventors' hypothesis, the inventors identified a similar break in the aforementioned tumor with NSD3/FGFR1-centered amplification that lacked a deleterious break in NSD3 (FIG. 3a). This tumor exhibited a highly covered tail-to-tail break close to the transcription start side of FGFR1, indicating an early event by break amplification, and an intra-chromosomal tail-to-tail break within FGFR1 to a non-coding region. The break deleted exons 1 to 7 of FGFR1 including the canonical ATG start codon (FIG. 3a, b). However, this sample revealed a 50% higher transcription of FGFR1 (18 Fragments Per Kilobase Million) compared to NSD3_{long} (12 Fragments Per Kilobase Million) and revealed transcription across the breakpoint. Furthermore, the inventors confirmed transcription of the rearranged gene by reverse transcriptase PCR using primers covering the break point followed by sequencing (FIG. 3c). Finally, the inventors identified an additional FGFR1-amplified adenosquamous carcinoma with a tail-to-tail break within FGFR1 (FIG. 3d). This break led to deletion of exons 1 to 8 of FGFR1 (FIG. 3e). Again, supporting the inventors' hypothesis of intact FGFR1 gene expression in the context of N-terminal deletions, the inventors found active transcription across the breakpoint (FIG. 3f). In this tumor, too, the inventors observed positive staining for pFGFR1 in immunochemistry. Thus, all 4 tail-to-tail breaks within FGFR1 led to genomic deletions of exons of the 5′ part of the gene (up to exon 8) accompanied by expression of N-terminally truncated versions of FGFR1 lacking the ectodomain, as well as amplification of the region encoding the transmembrane and kinase domains (exons 9-18).

The N-terminal part of FGFR1 (comprising the ectodomain) is responsible for ligand specificity, as well as receptor autoinhibition. Furthermore, exon 9 has an in-frame non-canonical ATG start codon and deletion of the whole FGFR1 ectodomain leads to ligand independent dimerization. The inventors therefore hypothesized that N-terminal deletion of the ectodomain of FGFR1 by intragenic tail-to-tail rearrangements might generate oncogenic versions of the kinase that cause FGFR1 dependency and thus, sensitivity to FGFR inhibition.

The inventors used immunoblotting to analyze whether N-terminal deletion of the ectodomain of FGFR1 by intragenic tail-to-tail rearrangements generates oncogenic versions of the kinase that cause FGFR1 dependency. Briefly, cells were washed with cold PBS and lysed in RIPA Lysis Buffer supplemented with protease (Roche) and phosphatase inhibitor (Calbiochem) cocktails. After 20 minutes of incubation on ice, lysates were centrifuged at 18,000 g for 25 minutes. Protein concentration in supernatants was measured using BCA Protein Assay (ThermoScientific). Equivalent amounts of protein (30-60 μg) were denatured and separated on 4-12% SDS-PAGE gels and after blotting on nitrocellulose membranes (Amersham Hybond-C Extra). The following antibodies were used for immunoblotting: β-actin (MP Bioscience); phospho-FGFR (Tyr653, Tyr654), phospho-AKT (Ser473), AKT, phospho-ERK, and total ERK, total FGFR1 (Abcam), and conjugated antibodies to rabbit and mouse (Millipore).

The inventors further observed that ectopic expression of ΔEC-FGFR1 in murine Ba/F3 cells led to IL3-independent cell growth. By contrast, overexpression of wild-type (wt) FGFR1 was not sufficient to induce IL-3 independence. Furthermore Ba/F3 cells expressing ΔEC-FGFR1 were highly sensitive to FGFR inhibition by the FGFR inhibitors, BG.J398 and AZD4547 (FIG. 3g, h, i).

In summery, the inventors discovered that 8p11-8p12 amplifications in squamous cell lung cancer are typically caused by Breakage Fusion Bridges. The mechanism of amplification induces head-to-head followed by tail-to-tail intra-chromosomal breaks within the 8p arm. Based on this mechanism, the inventors were able to reconstruct the observed 8p11-p12 copy number profile, taking all genomic breaks within the 8p-arm into account. Furthermore, tail-to-tail breaks close to the transcription start side of FGFR1 led to focal and high-amplitude amplification, centered on FGFR1/NSD3 and were associated with sensitivity to FGFR inhibition. The corresponding head-to-head break was frequently located within NSD3 (WHSC1L1), which led to deletion of exons 6 to 23, thereby deleting the catalytic SET domain of NSD3 (exons 19-20). Taking all 52 samples together, 8 out of 9 NSD3 breaks (89%) were detected in 18 samples with focal FGFR1 amplification driving expression of non-oncogenic NSD3short (exons 1-10). By contrast, in 4 FGFR1-amplified samples (8% of FGFR1 amplified samples used in this study) a tail-to-tail break occurred within the open reading frame (ORF) of FGFR1 deleting various portions of the gene ranging from exons 1-8. These breaks induced expression of a short and oncogenic version of FGFR1 lacking the ectodomain using a non-canonical in frame ATG start codon in exon 9. Mechanistically, ligand-independent dimerization by FGFR1 variants lacking the ectodomain has been previously reported. However, somatic genomic alterations that cause such variants have not been described to date.

To proof expression over the breakpoint, validate the tail-to-tail rearrangement, as well as the ΔEC-FGFR1 variant, the inventors generated cDNA from 1 μg total RNA of the sample Soo674 and its corresponding normal (provided from Elisabeth Brambilla, Grenoble). cDNA was generated using the SuperScripIII kit (Invirtrogen) following manufacturer's instructions followed by PCR using the break spanning primers 191_F1_S00674/201_R9_S00674 and 193_F2_S00674/202_R10_S00674. A head-to-head rearrangement was validated in this sample by a nested PCR using only one primer per PCR run (based on the palindromic nature of this break). In the first run the inventors used the primer 261_6_R1. In the second run (4 μl Template from first PCR) the inventors used the primer 262_6_R2. The expected band size was 794 base pairs (based on the whole genome sequencing data). cDNA of H1581 cells (100 ng) was used to amplify FGFR1 by attB-overhang primers and flip it into pDONR.221 using the BP-clonase (Invitrogen). Bacterial transformation of the competent E. coli strain DH5α (Invitrogen) was carried out according to the manufacturer's instructions. Single clones were sequenced from mini-preparation of plasmid DNA using the NucleoSpin Mini Kit (Machery Nagel). For midi-preparation of plasmid DNA the inventors used the NucleoBond Xtra Midi EF Kit (Machery Nagel). The different FGFR1 variants were generated by side using Gibson Assembly and the primers 234_F_ΔEC-21-FGFR1, 235_F_ΔEC-30-FGFR1, 236_F_ΔEC-85-FGFR1, 237_F_ΔEC-144-FGFR1, and 211_R_FGFR1_GA.

Conclusively, the inventors' findings suggest that intra-chromosomal tail-to-tail breaks in FGFR1—in some cases causing ectodomain-deficient versions of the kinase—may define squamous cell lung cancers with therapeutically relevant FGFR dependency. Patients with such rearrangements might benefit from treatment with FGFR inhibitors.

1. A method for identifying a subject suffering from cancer as a responder or non-responder to fibroblast growth factor receptor 1 (FGFR1) inhibitor therapy, the method comprising the steps of
a) Providing from the subject a biological sample comprising genetic material associated with the cancer,
b) Determining in the genetic material the presence or absence of a genetic alteration of the FGFR1 gene, wherein the genetic alteration is a copy number variation caused by a chromosomal break, such as a Breakage-Fusion-Bridge-like (BFB-like) mechanism, wherein the break is located within, or within close proximity of, the ORF of the FGFR1 gene;
wherein the presence of the genetic alteration in the FGFR1 gene in the biological sample of the subject indicates that the subject is a responder to FGFR1-inhibitor therapy.

2. The method according to claim 1, wherein the cancer is a solid cancer, preferably a cancer characterized by the expression of FGFR1, such as breast cancer, a gastric cancer, bladder cancer or lung cancer, and most preferably is Non-small cell lung cancer (NSCLC), such as squamous cell lung cancer (SQLC).

3. The method according to claim 1, wherein the subject is a human, preferably a human patient diagnosed with NSCLC, such as SQLC.

4. The method according to claim 1, wherein the biological sample comprises tumour cells of the subject, preferably wherein the biological sample is a tumour biopsy, or a sample of a resected primary tumour.

5. The method according to claim 1, wherein the FGFR1 inhibitor is a selective or unselective FGFR1 inhibitor, such as an inhibitor selected from the group consisting of NVP-BGJ398, AZD4547, TKI258, JNJ42756493, pemigatinib, lucitanib, Rogaratinib, Debio 1347, FIIN2, LY2874455, PRN1371 and ponatinib.

6. The method according to claim 1, wherein the genetic alteration is
a) a genetic modification of the FGFR1 locus resulting in a truncation of at least a part of an extracellular domain of FGFR1, and/or
b) a genetic copy number gain of a FGFR1 gene, or a modified variant thereof; and/or
c) a consecutive palindromic arrangement of the FGFR1 gene, or a partial FGFR1 gene; and/or
d) a genetic modification of the FGFR1 locus resulting in the translation initiation of a non-canonical ATG start-codon.

7. The method according to claim 1, wherein the genetic alteration is an alteration of the FGFR1 gene resulting in the expression of an ectodomain-deleted variant of FGFR1, preferably wherein the variant of FGFR1 is translated from a non-canonical ATG in exon 1 to exon 9 of FGFR1.

8. An inhibitor of FGFR1 for use in the treatment of cancer in a patient, wherein the cancer is characterized by a genome which comprises a genetic alteration in the FGFR1 gene comprising a consecutive palindromic copy number variation of the FGFR1 gene, or of a fragment of the FGFR1 gene, optionally wherein the cancer is a solid cancer, preferably a cancer characterized by the expression of FGFR1, such as breast cancer, a gastric cancer, bladder cancer or lung cancer, and most preferably is Non-small cell lung cancer (NSCLC), such as squamous cell lung cancer (SQLC).

9. The inhibitor of FGFR1 for use according to claim 8, wherein the inhibitor of FGFR1 is a small molecular inhibitor, an antigen binding protein, such as an antibody, or any antibody derivative, preferably which specifically binds to and inhibits FGFR, preferably FGFR1, optionally wherein the inhibitor of FGFR1 is a selective or unselective FGFR1 inhibitor, such as an inhibitor selected from the group consisting of NVP-BGJ398, AZD4547, TKI258, JNJ42756493, pemigatinib, lucitanib, Rogaratinib, Debio 1347, FIIN2, LY2874455, PRN1371 and ponatinib.

10. The inhibitor of FGFR1 for use according to claim 8, wherein the genetic alteration is a focal amplification of the FGFR1 gene, or of a fragment of the FGFR1 gene, optionally wherein the fragment of the FGFR1 gene is an expressible sequence and results in a FGFR1 protein variant, preferably an ectodomain-deleted FGFR1 protein variant.

11. The inhibitor of FGFR1 for use according to claim 8, wherein the treatment comprises a preliminary step of identifying the patient as a responder to an FGFR1 inhibitor therapy, wherein the preliminary step comprises a method according to any one of claims 1 to 7.

12. A method for the diagnosis, prognosis, stratification and/or monitoring of a therapy, of cancer in a subject, comprising the steps of:
a) Providing a biological sample from the subject, and
b) Determining in the biological sample the presence or absence of a chromosomal break, such as a Breakage-Fusion-Bridge-like (BFB-like) mechanism, for example a tail-to-tail BFB-like mechanism, wherein the break is located within, or within close proximity of, the ORF of the FGFR1 gene, an alteration of the mRNA-transcript of the FGFR1 gene, an alteration of the FGFR1 protein, an altered level of an mRNA-transcript of the FGFR1 gene, and/or an altered level of the FGFR1 protein, preferably wherein the extracellular domain of FGFR1 is deleted,
wherein the presence of the chromosomal break, the alteration of the mRNA-transcript of the FGFR1 gene, the alteration of the FGFR1 protein, a differential level of an mRNA-transcript of the FGFR1 gene, and/or a differential level of the FGFR1 protein as determined in step (b) compared to a healthy control or reference value is indicative for the presence of cancer in the subject.

13. The method according to claim 12, wherein the chromosomal break induces:
(i) a genetic modification of the FGFR1 locus resulting in a truncation of at least a part of an extracellular domain of FGFR1, and/or
(ii) a focal amplification of FGFR1, or of a fragment of the FGFR1 gene; and/or
(iii) a consecutive palindromic arrangement of the FGFR1 gene, or a partial FGFR1 gene; and/or
(iv) a genetic modification of the FGFR1 locus resulting in the translation initiation of a non-canonical ATG start-codon.

14. The method according to claim 12, wherein the genetic modification, and/or the alteration of the mRNA-transcript of the FGFR1 gene comprises a nucleic acid sequence according to any one of SEQ ID Nos: 2, 3, 10, 11, 15, 16, 20, 21, 25, 28, 31, 34, or 37, and/or wherein the altered FGFR1 protein comprises an amino acid sequence according to any one of SEQ ID Nos: 9, 14, 19, 24, 26, 29, 32, or 35.

15. The method according to claim 12, wherein the cancer is a solid cancer, preferably a cancer characterized by the expression of FGFR1, such as breast cancer, a gastric cancer, bladder cancer or lung cancer, and most preferably is Non-small cell lung cancer (NSCLC), such as squamous cell lung cancer (SQLC).
